(19) United States
(12) Patent Application Publication
(10) Pub. No.: US 2025/0009740 A1
(43) Pub. Date: Jan. 9, 2025

(54) METHODS AND COMPOSITIONS FOR TARGETING ALTERNATIVE METABOLISM ALONG WITH FLT3 INHIBITOR-MEDIATED ANTILEUKEMIC ACTIONS

(71) Applicant: Ohio State Innovation Foundation, Columbus, OH (US)

(72) Inventors: Rosa LAPALOMBELLA, Powell, OH (US); James BLACHLY, Upper Arlington, OH (US); Lindsey BRINTON, Charlottesville, VA (US)

(21) Appl. No.: 18/712,403

(22) PCT Filed: Nov. 22, 2022
(86) PCT No.: PCT/US2022/050759

§ 371 Date: May 22, 2024

Related U.S. Application Data
(60) Provisional application No. 63/281,934, filed on Nov. 22, 2021.

(51) Int. Cl. A61K 31/497; A61K 31/47; A61K 31/506; A61K 31/519; A61K 39/395; A61P 35/02; C12N 15/113

(52) U.S. Cl. CPC A61K 31/497; A61K 31/47; A61K 31/506; A61K 31/519; A61K 39/3955; A61P 35/02; C12N 15/1137; C12Y 207/10001; C12N 2310/14; C12N 2320/31

(57) ABSTRACT

It was found that FLT3-associated diseases could be treated by exposing an abnormal cell to a FLT3 inhibitor, wherein the method further comprises exposing the abnormal cell to a composition which promotes reactivation of glycolysis of the cell. Also contemplated are compositions for treating FLT3-associated disease, the composition comprising a combination of a reactivator of glycolysis and an inhibitor of FLT3.

### CROSS-REFERENCE TO RELATED APPLICATIONS

This application claims benefit of U.S. Provisional Application No. 63/281,934, filed Nov. 22, 2021, incorporated herein by reference in its entirety.

### BACKGROUND

Internal tandem duplication (ITD) and mutations within the tyrosine kinase domain (TKD) of fms-like tyrosine kinase 3 (FLT3) occur in 30% of acute myeloid leukemia (AML) cases^{1,2}. Presence of the FLT3 mutations at high variant allele frequency (Allelic Ratio; AR of >0.5) is associated with poor survival¹. Both mutations lead to a constitutively active receptor tyrosine kinase, causing prolonged signal transduction along cell survival and proliferative axes. In addition, FLT3-ITD has been shown to mediate metabolic reprograming by elevating aerobic glycolysis through upregulation of the mitochondrial hexokinase 2 (HK2). Therefore, FLT3-ITD leukemia cells are addicted to glycolysis and susceptible to pharmacological inhibition of glycolytic activity³.

Despite improved survival seen in FLT3-mutant AML patients treated with approved FLT3 inhibitors, midostaurin and gilteritinib, patients frequently experience relapse. The optimal use of these inhibitors in the upfront, relapse, and maintenance settings remains to be established⁴. Given the success of the highly selective FLT3 inhibitor, gilteritinib⁴, and its increasing adoption in the clinic, what is needed in the art are therapies which enhance the effectiveness of FLT3 treatments.

### SUMMARY

Disclosed herein is a method of treating FLT3-associated disease in a subject in need thereof, the method comprising exposing an abnormal cell to a FLT3 inhibitor, wherein the method further comprises exposing the abnormal cell to a composition which promotes reactivation of glycolysis of the cell.

Also disclosed herein is a composition for treating FLT3-associated disease, the composition comprising a combination of a reactivator of glycolysis and an inhibitor of FLT3.

### DESCRIPTION OF DRAWINGS

FIG. 1A-E shows CRISPR knockout screen reveals potential synergistic partners with gilteritinib. (A) Schematic overview of genome-wide CRISPR screen design. (B) Volcano plot segregating candidate hits into positively-(red) and negatively-selected (blue) genes (4 biological replicates per condition). (C) Contribution of each sgRNA to top hits. (D) Overlapping hits shared by gilteritinib and midostaurin screens. (E) Pathway enrichment analysis of top hits in negative selection by IPA.

FIG. 2A-I shows genetic depletion of CRISPR screen top hits, CDK9, PRMT5 or DHODH, but not CDK7, sensitizes AML cells to gilteritinib treatment. (A-D) Knockdown efficiency of selected targets by shRNA as detected by qPCR and Western blotting; GAPDH or β-actin serves as loading control. N=2. *p<0.05; **p<0.01; (E-H) Dose-response curves of shCDK9, shDHODH, shPRMT5 and shCDK7-stable MOLM-13 cells in response to 120-hour gilteritinib treatment. Cell viability was measured with MTS. Results are shown as mean±SEM of 4 technical replicates and 3-4 biological replicates. ***p-value<0.0001; ns=not significant. I) Knockdown of CDK9, PRMT5 or DHODH but not CDK7 with shRNA increases the frequency of apoptotic AML cell lines with gilteritinib. Parental, scrambled or gene-targeting shRNA-stable MOLM-13 cells were treated with 8 nM gileritinib for 120 hours and stained with Annexin V/PI for flow cytometry analysis. Data are shown as mean±SEM of % population from triplicates.

FIG. 3A-H shows RNA-seq analysis reveals distinct transcriptional signatures conferred by CDK9 or DHODH inactivation in combination with gilteritinib treatment. (A) PCA of transcriptomes of all combination and single arms over replicates. (B) Top panel: PCA plots of three biological replicates of gilteritinib-treated shCDK9-stable and 48 hr vehicle-treated scrambled shRNA-stable cells; Bottom panel: PCA plots of three biological replicates of gilteritinib-treated shDHODH-stable and 96 hr vehicle-treated scrambled shRNA-stable cells. (C-D) Heatmap representations of normalized read counts of top 25 downregulated and top 25 upregulated differentially expressed genes in shCDK9/gilteritinib and shDHODH/gilteritnib combination treatments. Different treatment groups are color-coded (purple: scrambled+vehicle; cyan: scrambled+gilteritinib; pink: shCDK9+vehicle or shDHODH+vehicle; green: shCDK9+gilteritinib or shDHODH+gilteritinib). (E-F) Volcano plots of selected treatment groups with respect to the corresponding scrambled/vehicle controls. Significantly downregulated and upregulated DEGs are highlighted in red. (G) Venn diagram showing the overlaps among top co-essential genes in CRISPR screen negative selection, top downregulated DEGs in shCDK9/gilteritinib combination RNA-seq and top downregulated DEGs in shDHODH/gilteritinib combination RNA-seq (CRISPR screen: FDR<0.25; RNA-seq: adj (p-value)<0.05). Pathway enrichment analysis of overlapped genes in three data sets is shown. (H) Upregulated DEGs and enriched pathways shared by shCDK9/gilteritinib combination RNA-seq and shDHODH/gilteritinib combination RNA-seq.

FIG. 4A-G shows genetic inhibition of CDK9 or DHODH in combination with gilteritinib alters multiple signature pathways. (A) GSEA plots of representative significantly downregulated and upregulated pathways in hallmark gene sets for shCDK9/gilteritinib vs scrambled/gilteritinib and scrambled/gilteritinib vs scrambled/vehicle comparisons. Combination treatment suppresses these pathways that are activated by gilteritinib treatment alone. (B) Cytoscape enrichment map of top gene programs in shCDK9/gilteritinib combination. Enriched GSEA gene sets are predicted with EnrichmentMap in CytoScape and depicted by orange and purple nodes, where purple nodes represent significantly upregulated pathways in combination treatment and orange nodes represent significantly downregulated pathways in combination treatment. Node size is proportional to the number of genes in each node, line thickness indicates the overlap of genes between nodes, and the theme of genes in each cluster is specified. Clustered gene programs are labeled. (C) Heatmap showing normalized read counts of genes in selected top enriched pathways predicted by GSEA across different treatment groups of shCDK9-mediated synergy. Selected genes are labeled. The hierarchical clustering of genes and samples was performed with Euclidean distance matrix and Ward's clustering method. (D) GSEA plots of representative significantly downregulated and upregulated pathways in hallmark gene sets for shDHODH/gilteritinib vs scrambled/vehicle and scrambled/gilteritinib vs scrambled/vehicle comparisons. Combination treatment suppresses these pathways that are activated by gilteritinib alone. (E) Cytoscape enrichment map of top gene programs in shDHODH/gilteritinib combination. (F) Heatmap showing normalized read counts of genes in selected top enriched pathways predicted by GSEA across different treatment groups of shDHODH-mediated synergy. Selected genes are labeled. The hierarchical clustering of genes and samples was performed with Euclidean distance matrix and Ward's clustering method. (G) Top glycolysis-related hits in positive selection of CRISPR screen upon gilteritinib treatment are highlighted in glycolysis pathway. Log (fold change) values of top 5 hits are listed.

FIG. 5A-F shows FLT3-ITD inhibition and ablation of identified co-essential genes synergistically inhibit the expressions of anti-apoptotic/pro-proliferative genes and cause metabolic rewiring. (A) The relative expressions of selected genes in pro-proliferation/anti-apoptosis, OXPHOS, purine de novo biosynthesis, mevalonate metabolism, glycolysis and glutamine transport pathways and PTK2, KRT18 and PRMT5 in scrambled, shCDK9, shPRMT5 and shDHODH-stable MOLM-13 in response to vehicle or 8 nM gilteritinib treatment were measure by real-time PCR with respect to GAPDH. Values are expressed as fold changes (mean±SEM, n=3) relative to vehicle-treated scrambled cells. *p<0.05; **p<0.01; ns=not significant. (B) Relative expressions of selected metabolic genes in scrambled, shCDK9, shPRMT5 and shDHODH-stable MOLM-13 in response to vehicle or 8 nM gilteritnib treatment at protein levels as revealed by Western blotting. GAPDH serves as loading control. Results are representative of duplicates. (C) Relative expressions of selected metabolic genes in MOLM-13 and MV4-11 cells treated with gilteritinib and dinaciclib at different dose combinations at protein levels as revealed by Western blotting. B-tubulin serves as loading control. Results are representative of duplicates. (D) PCA of metabolomics datasets of all combination and single groups over replicates. (E) Top enriched metabolic pathways as predicted by Mummichog analysis and GSEA analysis. The size of the circle is correlated with the amounts of metabolites being identified in the pathway. Three combined treatments share steroid biosynthesis and purine biosynthesis pathways. (F) The normalized abundances of selected basic and acidic metabolites across different treatment groups. The normalization is performed by multiplying compound ion abundance with scalar factor calculated with a median and mean deviation approach based on all the detected abundance. *p<0.05; ns=not significant.

FIG. 6A-D shows in vitro pharmacologic validation of synthetic lethal targets with gilteritinib. (A) Synergistic effect of pairwise dose combinations of gilteritinib and dinaciclib (CDKi), EPZ015666 (PRMT5i) or brequinar (DHODHi) on MOLM-13 and MV4-11 cells. For dinaciclib, cells were treated for 2 hours with the drug before it was washed off and all other drugs were incubated with cells for 48-96 hours. Cell viability was measured with MTS. HSA analysis was used to determine regions of synergy. (B) Brequinar and gilteritinib synergistically suppress cell proliferation. MOLM-13 and MV4-11 cells were treated with vehicle, 50 nM or 100 nM brequinar in combination with vehicle, 4 nM or 8 nM gilteritinib for 96 hours before cells were fixed and permeabilized for intracellular BV421-Ki67 staining and flow cytometry analysis. (C) Synergistic effect of gilteritinib and dinaciclib combination on AML patient samples carrying FLT3-ITD mutation. Primary cells were treated with vehicle, single agents or drug combination before cell apoptosis was measured with Annexin V/PI staining. Results are shown as mean±SEM of 3 biological replicates. (D) Gilteritinib in combination with either EPZ015666, dinaciclib or brequinar eliminate self-renewal potentials of FLT3-ITD AML primary cells while sparing healthy cells. Bone marrow cells from 3 FLT3-ITD patients, 2 FLT3-WT patients and 2 healthy donors were seeded into Methocult at 50,000 cells per condition with vehicle, single agents or drug combinations. The numbers of colonies were quantified for 1^{st} and 2^{nd} platings after 14 days post seeding. Results are shown as means of 2 biological replicates. * p<0.05 in comparison with EPZ015666, dinaciclib or brequinar alone.

FIG. 7A-D shows the combination therapy of dinaciclib and gilteritinib manifests superior efficacy in a FLT3-ITD AML xenograft model. (A) NCG mice were engrafted with MOLM-13 cells expressing luciferase and treated with vehicle, 10 mg/kg dinaciclib weekly, 30 mg/kg gilteritinib daily, or dinaciclib/gilteritinib combination. (B) IVIS imaging shows changes in luciferase signal over six weeks. (C) Kaplan-Meier curves of the mouse survival times in different treatment groups. ***p-value<0.001; ****p-value<0.0001. (D) Combination therapy significantly reduced spleen size and weight of engrafted mice. Statistical significance of differences in spleen weights between groups were estimated using analysis of variance (ANOVA) methods. p-values have been adjusted for multiple comparisons using Holm's procedure. Representative images of excised spleens are shown with scales.

FIG. 8 shows Gini index for evenness of sgRNA reads, sgRNA with zero reads and mapping ratio for Day0, DMSO and gilteritinib samples.

FIG. 9 shows visualization of positively-(red) and negatively-selected (blue) genes on Acute Myeloid Leukemia FLT3 signaling map. Strength of selection is represented by color saturation.

FIG. 10 shows combined treatment of shCDK9, shDHODH or shPRMT5 and gilteritinib leads to mitochondrial dysfunction in cell lines. shCDK9-, shPRMT5- or shDHODH-stable MOLM-13 cells were treated with vehicle control, 8 nM gilteritinib, or 12 nM gilteritinib for 48 or 96 hours before being stained by TMRM in combination with Annexin V-FITC followed by flow cytometric analysis. n=2.

FIG. 11A-F shows a Log 2FC preranked lists of DEGs of indicated comparisons were employed to run GSEA against the Hallmark gene sets. Unsupervised hierarchical clustering of normalized enrichment scores (NES) was used to generate a comprehensive heatmap representation of the functional transcriptional outputs of the (A) CDK9− and (B) DHODH-related treatment comparison sets. (C) Top enriched pathways of DEGs of shCDK9+gilteritinib vs scrambled+vehicle comparison (FDR<0.05 and LFC>2.0) predicted by IPA. Orange:

downregulated; Blue: upregulated. (D) GSEA plots of chromosome segregation which is one of the top significantly downregulated pathways in C5 gene sets for shCDK9+gilteritinib vs scrambled+vehicle comparison. (E) Top enriched pathways of DEGs of shDHODH+gilteritinib vs scrambled+vehicle comparison (FDR<0.05 and LFC>2.0) predicted by IPA. (F) GSEA plots of steroid metabolism which is one of the top significantly downregulated pathways in C5 gene sets for shDHODH+gilteritinib vs scrambled+vehicle comparison.

FIG. 12 shows GSEA of shCDK9 and gilteritinib treatments in MOLM13 cells. Individual GSEA plots for top 3 downregulated and 2 upregulated gene-sets are shown for all four treatment groups.

FIG. 13 shows the heatmaps showing the normalized read counts of gene transcripts of Myc pathway, fatty acid metabolism and OXPHOS pathway in the leading edge subsets across comparisons in shCDK9-mediated synergy.

FIG. 14A-B shows a cytoscape enrichment map of top gene programs in (A) shCDK9/gilteritinib vs scramble/gilteritinib comparison and (B) shDHODH/gilteritinib vs scramble/gilteritinib combination.

FIG. 15 shows GSEA of shDHODH and gilteritinib treatments in MOLM13 cells. Individual GSEA plots for top 3 downregulated and 1˜2 upregulated gene-sets are shown for all four treatment groups.

FIG. 16 shows the heatmaps showing the normalized read counts of gene transcripts of Cholesterol homeostasis, fatty acid metabolism and OXPHOS pathways in the leading edge subsets across comparisons in shDHODH-mediated synergy.

FIG. 17 shows inhibition of glutaminolysis sensitizes AML cells to gilteritinib treatment. Proliferation assay of a range of doses of midostaurin or gilteritinib with telaglenstat on MOLM-13 cells treated for 48 hours. Highest single agent (HSA) analysis was used to determine regions of synergy.

FIG. 18A-D shows heatmaps showing the abundances of metabolites in (A-D) scrambled/vehicle, scrambled/gilteritinib, shCDK9/gilteritinib, shPRMT5/gilteritinib and shDHODH/gilteritinib.

FIG. 19 shows schematic illustration of the mechanism of CDK9i/gilteritinib, DHODHi/gilteritinib and PRMT5i/gilteritinib synergism.

### DETAILED DESCRIPTION

### Definitions

Unless otherwise defined below, the terms used in the present invention shall be understood in accordance with the common meaning known to the person skilled in the art.

Each publication, patent application, patent, and other reference cited herein is incorporated by reference in its entirety to the extent that it is not inconsistent with the present invention. References are indicated by their reference numbers and their corresponding reference details which are provided in the “references” section.

The term “prophylactically effective amount” refers to an amount of an active compound or pharmaceutical agent that inhibits or delays in a subject the onset of a disorder as being sought by a researcher, veterinarian, medical doctor or other clinician.

The term “therapeutically effective amount” as used herein, refers to an amount of active compound or pharmaceutical agent that elicits the biological or medicinal response in a subject that is being sought by a researcher, veterinarian, medical doctor or other clinician, which includes alleviation of the symptoms of the disease or disorder being treated.

Methods are known in the art for determining therapeutically and prophylactically effective doses for the instant pharmaceutical composition(s). As used herein, the term “composition” is intended to encompass a product comprising the specified ingredients in the specified amounts, as well as any product which results, directly or indirectly, from combinations of the specified ingredients in the specified amounts.

As used herein, the terms “disorders related to FLT3”. or “disorders related to FLT3 receptor”, or “disorders related to FLT3 receptor tyrosine kinase” shall include diseases associated with or implicating FLT3 activity, for example, the overactivity of FLT3, and conditions that accompany with these diseases. The term “overactivity of FLT3” refers to either 1) FLT3 expression in cells which normally do not express FLT3; 2) FLT3 expression by cells which normally do not express FLT3; 3) increased FLT3 expression leading to unwanted cell proliferation; or 4) mutations leading to constitutive activation of FLT3. Examples of “disorders related to FLT3” include disorders resulting from over stimulation of FLT3 due to abnormally high amount of FLT3 or mutations in FLT3, or disorders resulting from abnormally high amount of FLT3 activity due to abnormally high amount of FLT3 or mutations in FLT3. It is known that overactivity of FLT3 has been implicated in the pathogenesis of a number of diseases, including the cell proliferative disorders, neoplastic disorders and cancers listed below.

The term “cell proliferative disorders” refers to unwanted cell proliferation of one or more subset of cells in a multicellular organism resulting in harm (i.e., discomfort or decreased life expectancy) to the multicellular organisms. Cell proliferative disorders can occur in different types of animals and humans. For example, as used herein “cell proliferative disorders” include neoplastic disorders and other cell proliferative disorders.

As used herein, a “neoplastic disorder” refers to a tumor resulting from abnormal or uncontrolled cellular growth. Examples of neoplastic disorders include, but are not limited to, hematopoietic disorders such as, for instance, the myeloproliferative disorders, such as thrombocythemia, essential thrombocytosis (ET), angiogenic myeloid metaplasia, myelofibrosis (MF), myelofibrosis with myeloid metaplasia (MMM), chronic idiopathic myelofibrosis (IMF), polycythemia vera (PV), the cytopenias, and pre-malignant myelodysplastic syndromes; cancers such as glioma cancers, lung cancers, breast cancers, colorectal cancers, prostate cancers, gastric cancers, esophageal cancers, colon cancers, pancreatic cancers, ovarian cancers, and hematoglogical malignancies, including myelodysplasia, multiple myeloma, leukemias and lymphomas. Examples of hematological malignancies include, for instance, leukemias, lymphomas (non-Hodgkin's lymphoma), HodgMn's disease (also called Hodgkin's lymphoma), and myeloma ˜ for instance, acute lymphocytic leukemia (ALL), acute myeloid leukemia (AML), acute promyelocyte leukemia (APL), chronic lymphocytic leukemia (CLL), chronic myeloid leukemia (CML), ‘chronic neutrophilic leukemia (CNL), acute undifferentiated leukemia (AUL), anaplastic large-cell lymphoma (ALCL), prolymphocyte leukemia (PML), juvenile myelomonocyctic leukemia (JMML), adult T-cell ALL, AML with trilineage myelodysplasia (AML/TMDS), mixed lineage leukemia (MLL), myelodysplastic syndromes (MDSs), myeloproliferative disorders (MPD), and multiple myeloma, (MM).

As used herein, “chemotherapy” refers to a therapy involving a chemotherapeutic agent. A variety of chemotherapeutic agents may be used in the multiple component treatment methods disclosed herein. Chemotherapeutic agents contemplated as exemplary, include, but are not limited to: platinum compounds (e.g., cisplatin, carboplatin, oxaliplatin); taxane compounds (e.g., paclitaxcel, docetaxol); campotothecin compounds (irinotecan, topotecan); vinca alkaloids (e.g., vincristine, vinblastine, vinorelbine); anti-tumor nucleoside derivatives (e.g., 5-fluorouracil, leucovorin, gemcitabine, capecitabine); alkylating agents (e.g., cyclophosphamide, carmustine, lomustine, thiotepa); epipodophyllotoxins/podophyllotoxins (e.g. etoposide, teniposide); aromatase inhibitors (e.g., anastrozole, letrozole, exemestane); anti-estrogen compounds (e.g., tamoxifen, fulvestrant), antifolates (e.g., premetrexed disodium); hypomethylating agents (e.g., azacitidine); biologies (e.g., gemtuzamab, cetuximab, rituximab, pertuzumab, trastuzumab, bevacizumab, erlotinib); antibiotics/anthracyclines (e.g. idarubicin, actinomycin D, bleomycin, daunorubicin, doxorubicin, mitomycin C, dactinomycin, carminomycin, daunomycin); antimetabolites (e.g., aminopterin, clofarabine, cytosine arabinoside, methotrexate); tubulin-binding agents (e.g. combretastatin, colchicine, nocodazole); topoisomerase inhibitors (e.g., camptothecin). Further useful agents include verapamil, a calcium antagonist found to be useful in combination with antineoplastic agents to establish chemosensitivity in tumor cells resistant to accepted chemotherapeutic agents and to potentiate the efficacy of such compounds in drug-sensitive malignancies. See Simpson W G, The calcium channel blocker verapamil and cancer chemotherapy. Cell Calcium. 1985 December; 6 (6): 449-67. Additionally, yet to emerge chemotherapeutic agents are contemplated as being useful in combination with the compound of the present invention.

A “kinase inhibitor” as referred to herein is a molecular compound which inhibits one or more kinase(s) by binding to said kinase(s) and exerting an antagonistic effect on said kinase. A kinase inhibitor is capable of binding to one or more kinase species, upon which the kinase activity of the one or more kinase is reduced. A kinase inhibitor as described herein is typically a small molecule, wherein a small molecule is a molecular compound of low molecular weight (typically less than 1 kDa) and size (typically smaller than 1 nM).

In one embodiment, the kinase inhibitor is a multikinase inhibitor. As used herein, a “multikinase inhibitor” is a kinase inhibitor capable of inhibiting more than one type of kinase. In a preferred embodiment, the kinase inhibitor is a tyrosine kinase inhibitor. In another preferred embodiment, the kinase inhibitor is an FLT3 inhibitor. In a more preferred embodiment, the kinase inhibitor is an FLT3 kinase inhibitor selected from the group consisting of gilteritinib, crenolanib, midostaurin, and quizartinib.

The terms “K_{D}” or “K_{D} value” relate to the equilibrium dissociation constant as known in the art. In the context of the present invention, these terms relate to the equilibrium dissociation constant of a targeting agent with respect to a particular antigen of interest (e.g. FLT3, PRMT5, CDK9, DHODH, BCL2, or XPO1). The equilibrium dissociation constant is a measure of the propensity of a complex (e.g. an antigen-targeting agent complex) to reversibly dissociate into its components (e.g. the antigen and the targeting agent). Methods to determine Kp values are known in art.

An “inhibitor” as described herein is a targeting agent that is capable of binding specifically to its target and reducing activity of the target molecule. This reduction, or inhibition, of activity of the target molecule can be by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any amount in-between these values.

Terms such as “inhibition of growth of cells” as used herein mean the effect of causing a decrease in cell number. Preferably, this can be caused by cytotoxicity through necrosis or apopotisis, or this can be caused by inhibiting or stopping proliferation. A “growth inhibiting effect” as used herein means that a substance, molecule, compound, composition or agent has a growth inhibiting effect on the cells as compared to a situation where said substance, molecule, compound, composition, or agent is not present. Cell growth inhibition can be measured by various common methods and assays known in the art.

The term “antibody” as used herein refers to any functional antibody that is capable of specific binding to the antigen of interest. Without particular limitation, the term antibody encompasses antibodies from any appropriate source species, including avian such as chicken and mammalian such as mouse, goat, non-human primate and human. Preferably, the antibody is a humanized antibody. Humanized antibodies are antibodies which contain human sequences and a minor portion of non-human sequences which confer binding specificity to an antigen of interest (e.g. human FLT3, PRMT5, CDK9, DHODH, BCL2, or XPO1). The antibody is preferably a monoclonal antibody which can be prepared by methods well-known in the art. The term antibody encompasses an IgG-1, -2, -3, or -4, IgE, IgA, IgM, or IgD isotype antibody. The term antibody encompasses monomeric antibodies (such as IgD, IgE, IgG) or oligomeric antibodies (such as IgA or IgM). The term antibody also encompasses, without particular limitations, isolated antibodies and modified antibodies such as genetically engineered antibodies, e.g. chimeric antibodies or bispecific antibodies.

An antibody fragment or fragment of an antibody as used herein refers to a portion of an antibody that retains the capability of the antibody to specifically bind to the antigen (e.g. human FLT3, PRMT5, CDK9, DHODH, BCL2, or XPO1). This capability can, for instance, be determined by determining the capability of the antigen-binding portion to compete with the antibody for specific binding to the antigen by methods known in the art. Without particular limitation, the antibody fragment can be produced by any suitable method known in the art, including recombinant DNA methods and preparation by chemical or enzymatic fragmentation of antibodies. Antibody fragments may be Fab fragments, F(ab′) fragments, F(ab′)2 fragments, single chain antibodies (scFv), single-domain antibodies, diabodies or any other portion(s) of the antibody that retain the capability of the antibody to specifically bind to the antigen.

An “antibody” (e.g. a monoclonal antibody) or “a fragment thereof” as described herein may have been derivatized or be linked to a different molecule. For example, molecules that may be linked to the antibody are other proteins (e.g. other antibodies), a molecular label (e.g. a fluorescent, luminescent, colored or radioactive molecule), a pharmaceutical and/or a toxic agent. The antibody or antigen-binding portion may be linked directly (e.g. in form of a fusion between two proteins), or via a linker molecule (e.g. any suitable type of chemical linker known in the art).

Terms such as “treatment of cancer” or “treating cancer” according to the present invention refer to a therapeutic treatment. An assessment of whether or not a therapeutic treatment works can, for instance, be made by assessing whether the treatment inhibits cancer growth in the treated patient or patients. Preferably, the inhibition is statistically significant as assessed by appropriate statistical tests which are known in the art. Inhibition of cancer growth may be assessed by comparing cancer growth in a group of patients treated in accordance with the present invention to a control group of untreated patients, or by comparing a group of patients that receive a standard cancer treatment of the art plus a treatment according to the invention with a control group of patients that only receive a standard cancer treatment of the art. Such studies for assessing the inhibition of cancer growth are designed in accordance with accepted standards for clinical studies, e.g. double-blinded, randomized studies with sufficient statistical power. The term “treating cancer” includes an inhibition of cancer growth where the cancer growth is inhibited partially (i.e. where the cancer growth in the patient is delayed compared to the control group of patients), an inhibition where the cancer growth is inhibited completely (i.e. where the cancer growth in the patient is stopped), and an inhibition where cancer growth is reversed (i.e. the cancer shrinks). An assessment of whether or not a therapeutic treatment works can be made based on known clinical indicators of cancer progression.

A treatment of cancer according to the present invention does not exclude that additional or secondary therapeutic benefits also occur in patients. For example, an additional or secondary benefit may be an enhancement of engraftment of transplanted hematopoietic stem cells that is carried out prior to, concurrently to, or after the treatment of cancer. However, it is understood that the primary treatment for which protection is sought is for treating the cancer itself, and any secondary or additional effects only reflect optional, additional advantages of the treatment of cancer growth.

The treatment of cancer according to the invention can be a first-line therapy, a second-line therapy, a third-line therapy, or a fourth-line therapy. The treatment can also be a therapy that is beyond is beyond fourth-line therapy. The meaning of these terms is known in the art and in accordance with the terminology that is commonly used by the US National Cancer Institute.

The term “capable of binding” as used herein refers to the capability to form a complex with a molecule that is to be bound (e.g. FLT3, PRMT5, CDK9, DHODH, BCL2, or XPO1). Binding typically occurs non-covalently by intermolecular forces, such as ionic bonds, hydrogen bonds and Van der Waals forces and is typically reversible. Various methods and assays to determine binding capability are known in the art. Binding is usually a binding with high affinity, wherein the affinity as measured in Kp values is preferably less than 1 μM, more preferably less than 100 nM, even more preferably less than 10 nM, even more preferably less than 1 nM, even more preferably less than 100 pM, even more preferably less than 10 pM, even more preferably less than 1 pM.

As used herein, each occurrence of terms such as “comprising” or “comprises” may optionally be substituted with “consisting of” or “consists of”.

A pharmaceutically acceptable carrier, including any suitable diluent or, can be used herein as known in the art. As used herein, the term “pharmaceutically acceptable” means being approved by a regulatory agency of the Federal or a state government or listed in the U.S. Pharmacopia, European Pharmacopia or other generally recognized pharmacopia for use in mammals, and more particularly in humans. Pharmaceutically acceptable carriers include, but are not limited to, saline, buffered saline, dextrose, water, glycerol, sterile isotonic aqueous buffer, and combinations thereof. It will be understood that the formulation will be appropriately adapted to suit the mode of administration.

Compositions and formulations in accordance with the present invention are prepared in accordance with known standards for the preparation of pharmaceutical compositions and formulations. For instance, the compositions and formulations are prepared in a way that they can be stored and administered appropriately, e.g. by using pharmaceutically acceptable components such as carriers, excipients or stabilizers. Such pharmaceutically acceptable components are not toxic in the amounts used when administering the pharmaceutical composition or formulation to a patient. The pharmaceutical acceptable components added to the pharmaceutical compositions or formulations may depend on the chemical nature of the inhibitor and targeting agent present in the composition or formulation (depend on whether the targeting agent is e.g. an antibody or fragment thereof or a cell expressing a chimeric antigen receptor), the particular intended use of the pharmaceutical compositions and the route of administration.

### Methods

Disclosed herein is a method of treating FLT3-associated disease in a subject in need thereof, the method comprising exposing an abnormal cell to a FLT3 inhibitor, wherein the method further comprises exposing the abnormal cell to a composition which promotes reactivation of glycolysis of the cell.

FLT3 is a class III receptor tyrosine kinase that plays an important role in normal hematopoiesis and mutations thereof have been associated with acute myeloid leukemia (AML) and acute lymphoblastic leukemia (ALL), as well as other disorders. Recent large-scale genomic sequencing efforts have confirmed that FLT3 is the most commonly mutated gene in human AML, with about 20% of mutations consisting of internal tandem duplication (ITD) mutations in the juxtamembrane domain (JMD) and with an additional subset consisting of point mutations in the FL73 tyrosine kinase domain (TKD), commonly at the activation loop residue D835 (Smith C C. Disease diversity and FLT3 mutations. Proc Natl Acad Sci USA. 2013 Dec. 24; 110 (52): 20860-1). Therefore, specifically contemplated herein is a FLT3-associated disease is caused by an alteration in the FLT3 gene, such as IDT, JMD, and TKD.

The currently available FLT3 inhibitors are tyrosine kinase inhibitors (TKI) classified into first and next generation inhibitors based on their potency and specificity for FLT3 and their associated downstream targets. Small molecule inhibitors of FLT3 include, but are not limited to, sunitinib, lestaurtinib, ponatinib, tandutinib, sorafenib, midostaurin, crenolanib, quizaritinib, FF-10101, HM43239, and gilteritinib. One of skill in the art can readily ascertain how to administer these FLT3 inhibitors, and specific guidance is given in Antar et al. (Antar, A. I. et al. FLT3 inhibitors in acute myeloid leukemia: ten frequently asked questions. Leukemia 34, 682-696 (2020), which is incorporated by reference in its entirety for its teaching concerning inhibition of FLT3 by small molecule inhibitors.

By “FLT3 inhibitor” is meant that the inhibitor reduces the activity of the protein. The FLT3 inhibitor can inhibit activity of FLT3 by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any amount in between. More specifically, the inhibitor of FLT3 can reduce glycolysis by the cell by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any amount in between.

The FLT3 inhibitor can also be RNA-based therapy, such as RNAi, siRNA, or miRNA. For example, Walters et al. (Walters D K et al. RNAi-induced down-regulation of FLT3 expression in AML cell lines increases sensitivity to MLN518. Blood. 2005 Apr. 1; 105 (7): 2952-4), which is incorporated by reference in its entirety for its discussion regarding RNA-based inhibition of FLT3, discuss the use of siRNA to downregulate FLT3. The FLT3 inhibitor can also be an antibody. Anti-FLT3 antibodies are known in the art (Piloto et al. Cancer Res. 2005 Feb. 15; 65 (4): 1514-22), and are contemplated herein, such as the IMC-EB10 antibody.

FLT3 inhibition shifts metabolic dependency from aerobic glycolysis to alternative pathways, such as oxidative phosphorylation (OXPHOS), mevalonate metabolism, and/or purine biosynthesis, thus rendering the cells which are now dependent upon alternative metabolic pathways, sensitive to inhibition. By inhibiting the alternative pathway, cells must rely again on glycolysis, which is inhibited by FLT3, thereby making FLT3 inhibition considerably more effective (Example 1). Disclosed herein is that inhibition of protein arginine N-methyltransferase 5 (PRMT5), cyclin dependent kinase 9 (CDK9), and/or dihydroorotate dehydrogenase (DHODH), which are associated with alternative metabolic pathways, plus inhibition of FLT3, such as by gilteritenib treatment, cooperatively shuts down cell function and therefore is an effective cancer treatment.

Therefore, contemplated herein is inhibition of both FLT3 and PRMT5, CDK9, and/or DHODH. The FLT3 inhibitor and inhibitor of PRMT5, CDK9, and/or DHODH can be given simultaneously, or the FLT3 inhibitor can be given prior to or after the inhibitor(s) of PRMT5, CDK9, and/or DHODH. For example, the FLT3 inhibitor can be given 6, 12, 18, 24, 30, 36, 42, or 48 hours prior, or 3, 4, 5, 6, 7, 8, 9, 10, 11, 12, 13, or 14 days prior to treatment with the inhibitor(s) of PRMT5, CDK9, and/or DHODH. The FLT3 inhibitor can also be given after the inhibitor(s) of PRMT5, CDK9, and/or DHODH. For example, the FLT3 inhibitor can be given 6, 12, 18, 24, 30, 36, 42, or 48 hours after, or 3, 4, 5, 6, 7, 8, 9, 10, 11, 12, 13, or 14 days after, treatment with the inhibitor(s) of PRMT5, CDK9, and/or DHODH.

Inhibitors of PRMT5, CDK9, and DHODH are known to those of skill in the art. For example, inhibitors of DHODH include, but are not limited to, brequinar, BAY 2402234, BRQ, leflunomide, teriflunomide, and ALASN003. Inhibitors of CDK9 include, but are not limited to, CAS 140651-18-9, dinaciclib, flavopiridol, VIP152, AZD-4573, and SNS-032. PRMT5 inhibitors include, but are not limited to, EPZ015666, GSK591, MRTX1719, LLY-283, PRT811, and PF-06939999. Other small molecule inhibitors, RNA inhibitors, and antibody inhibitors are known to those of skill in the art.

In addition to inhibition of PRMT5, CDK9, and/or DHODH, additional proteins can also be inhibited. Examples include, but are not limited to, inhibition of B-cell lymphoma 2 (BCL2) and/or exportin 1 (XPO1). Inhibitors of these proteins/genes which encode them are known in the art.

By “PRMT5, CDK9, and/or DHODH inhibitor” is meant that the inhibitor reduces the activity of the protein. The PRMT5, CDK9, and/or DHODH inhibitor can inhibit activity of FLT3 by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any amount in between. More specifically, the inhibitor of PRMT5, CDK9, and/or DHODH can inhibit use of the alternative metabolic pathway by the cell by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any amount in between.

When treating a cancer patient, the addition of PRMT5, CDK9, and/or DHODH inhibition to FLT3 treatment can serve to prolong the patient's life or enhance the quality of their life. When used to prolong life, the synergistic effect of the combined treatment can prolong the life of the subject by 1, 2, 3, 4, 5, 6, 7, 8, 9, 10, 11, or 12 months, or 18 months, or 2, 3, 4, 5, 6, 7, 8, 9, or 10 years or more.

### Compositions

Also disclosed are compositions for treating FLT3-related diseases. These compositions include the combination of a FLT3 inhibitor and an inhibitor of one or more of PRMT5, CDK9, and DHODH. In addition to these inhibitors, the composition can also comprise an inhibitor of BCL2 and/or XPO1. This combination provides a synergistic effect which increases the effectiveness of the FLT3 inhibitor. This effectiveness can be increased by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or by 2, 3, 4, 5, 6, 7, 8, 9, or 10 fold, or any amount in between or above these values, as compared to the FLT3 inhibitor being used alone. This can be measured by a reduction in cancer cells, cancer markers, or growth in cancer cells, or example.

In another embodiment of the present invention, the FLT3 kinase inhibitor and PRMT5, CDK9, and/or DHODH inhibitor may be administered in combination with gene therapy. As used herein, “gene therapy” refers to a therapy targeting on particular genes involved in tumor development. Possible gene therapy strategies include the restoration of defective cancer-inhibitory genes, cell transduction or transfection with antisense DNA corresponding to genes coding for growth factors and their receptors, RNA-based strategies such as ribozymes, RNA decoys, antisense messenger RNAs and small interfering RNA-(SiRNA) molecules and the so-called ‘suicide genes’. In other embodiments of this invention, the FLT3 kinase inhibitor and PRMT5, CDK9, and/or DHODH inhibitor may be administered in combination with immunotherapy. As used herein, “immunotherapy” refers to a therapy targeting particular protein involved in tumor development via antibodies specific to such protein. For example, monoclonal antibodies against vascular endothelial growth factor have been used in treating cancers.

Where one or more additional chemotherapeutic agent(s) are used in conjunction with the FLT3 kinase inhibitor and PRMT5, CDK9, and/or DHODH inhibitor, the additional chemotherapeutic agent(s), the FLT3 kinase inhibitor and the PRMT5, CDK9, and/or DHODH inhibitor may be administered simultaneously (e.g. in separate or unitary compositions) sequentially in any order, at approximately the same time, or on separate dosing schedules. In the latter case, the pharmaceuticals will be administered within a period and in an amount and manner that is sufficient to ensure that an advantageous and synergistic effect is achieved. It will be appreciated that the preferred method and order of administration and the respective dosage amounts and regimes for the additional chemotherapeutic agent(s) will depend on the particular chemotherapeutic agent(s) being administered in conjunction with the FLT3 kinase inhibitor and PRMT5, CDK9, and/or DHODH inhibitor, their route of administration, the particular tumor being treated and the particular host being treated. As will be understood by those of ordinary skill in the art, the appropriate doses of the additional chemotherapeutic agent(s) will be generally similar to or less than those already employed in clinical therapies wherein the chemotherapeutics are administered alone or in combination with other chemotherapeutics.

The optimum method and order of administration and the dosage amounts and regime can be readily determined by those skilled in the art using conventional methods and in view of the information set out herein.

The FLT3 kinase inhibitor and PRMT5, CDK9, and/or DHODH inhibitor can be administered to a subject systemically, for example, intravenously, orally, subcutaneously, intramuscular, intradermal, or parenterally. The FLT3 kinase inhibitor and PRMT5, CDK9, and/or DHODH inhibitor can also be administered to a subject locally. Non-limiting examples of local delivery systems include the use of intraluminal medical devices that include intravascular drug delivery catheters, wires, pharmacological stents and endoluminal paving.

The FLT3 kinase inhibitor and PRMT5, CDK9, and/or DHODH inhibitor can further be administered to a subject in combination with a targeting agent to achieve high local concentration of the FLT3 kinase inhibitor and PRMT5, CDK9, and/or DHODH inhibitor at the target site. In addition, the FLT3 kinase inhibitor PRMT5, CDK9, and/or DHODH inhibitor may be formulated for fast-release or slow-release with the objective of maintaining the drugs or agents in contact with target tissues for a period ranging from hours to weeks.

The separate pharmaceutical compositions comprising the FLT3 kinase inhibitor in association with a pharmaceutically acceptable carrier, and the PRMT5, CDK9, and/or DHODH inhibitor in association with a pharmaceutically acceptable carrier may contain between about 0.1 mg and 1000 mg, preferably about 100 to 500 mg, of the individual agents compound, and may be constituted into any form suitable for the mode of administration selected.

The unitary pharmaceutical composition comprising the FLT3 kinase inhibitor and PRMT5, CDK9, and/or DHODH inhibitor in association with a pharmaceutically acceptable carrier may contain between about 0.1 mg and 1000 mg, preferably about 100 to 500 mg, of the compound, and may be constituted into any form suitable for the mode of administration selected.

The phrases “pharmaceutically acceptable” refer to molecular entities and compositions that do not produce an adverse, allergic or other untoward reaction when administered to an animal, or a human, as appropriate. Veterinary uses are equally included within the invention and “pharmaceutically acceptable” formulations include formulations for both clinical and/or veterinary use.

Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixirs, emulsions, and suspensions. Forms' useful for parenteral administration include sterile solutions, emulsions and suspensions.

The pharmaceutical compositions of the present invention, whether unitary or separate, may be formulated for slow release of the FLT3 kinase inhibitor and PRMT5, CDK9, and/or DHODH inhibitor. Such a composition, unitary or separate, includes a slow release carrier (typically, a polymeric carrier) and one, or in the case of the unitary composition, both, of the FLT3 kinase inhibitor and PRMT5, CDK9, and/or DHODH inhibitor.

Slow release biodegradable carriers are well known in the art. These are materials that may form particles that capture therein an active compound(s) and slowly degrade/dissolve under a suitable environment (e.g., aqueous, acidic, basic, etc) and thereby degrade/dissolve in body fluids and release the active compound(s) therein. The particles are preferably nanoparticles (i.e., in the range of about 1 to 500 nm in diameter, preferably about 50-200 nm in diameter, and most preferably about 100 nm in diameter).

### Example 1: Targeting OXPHOS-Purine/Mevalonate Metabolism as the Nexus of FLT3 Inhibitor-Mediated Synergistic Antileukemic Actions

Disclosed herein are protein arginine N-methyltransferase 5 (PRMT5), cyclin dependent kinase 9 (CDK9), and dihydroorotate dehydrogenase (DHODH) as novel synthetic lethal partners in AML with gilteritinib treatment. Using both genetic and pharmacologic approaches, the co-essential nature of these genes was recapitulated in combination with gilteritinib treatment in FLT3-ITD cell lines and patient samples. By using RNA-seq and metabolomics, it was shown that the knockdown of CDK9, PRMT5 or DHODH plus gilteritenib treatment cooperatively shut down oxidative phosphorylation (OXPHOS), purine biosynthesis and the mevalonate pathway. Enrichment of sgRNAs targeting 28 glycolytic genes in cells treated with gilteritinib in positive selection screen was shown. This showed a metabolic adaption of the leukemic cells whereby they switch to OXPHOS from aerobic glycolysis to develop resistance to gilteritinib, a central metabolic pathway targeted by different synthetic lethal treatments to re-sensitize leukemic cells to gilteritinib treatment.

As a proof of concept, a combinatorial approach in vivo was performed, utilizing the 1/2/5/9CDK inhibitor, dinaciclib, which has been used in conjunction with venetoclax in clinical trials for relapsed/refractory AML. The human FLT3-ITD AML cell line xenograft model showed a promising survival benefit of dinaciclib/gilteritinib over monotherapies, showing that this combination can improve the outcome of AML patients with FLT3 mutations.

### Materials and Methods

### Cell Culture

Cells were cultured at 37° C. with 5% CO₂ in RPMI 1640 (Gibco) for MOLM-13 and MV4-11 (DSMZ, Germany) or DMEM (Gibco) for HEK293FT (Life Technologies, Carlsbad, CA), all supplemented with 10% FBS and 1% penicillin/streptomycin/L-glutamine (Gibco). Cell lines were validated via short tandem repeat analysis by The Ohio State University Genomic Services Core, routinely tested for mycoplasma contamination (Universal Mycoplasma Detection Kit, ATCC 30-1012K), and were discarded after passage twenty.

### Genome-Wide Loss-of-Function Screening

The human Brunello CRISPR knockout library was a gift from David Root and John Doench (Addgene #73179). The library was amplified and lentiviral particles were produced as previously described^{6,7}.

Animal Studies All animal studies were carried out under protocols approved by The Ohio State University Institutional Animal Care and Use Committee (IACUC). 1×10⁵ MOLM-13 luciferase cells were injected via tail vein into male NOD-Prkdc^{em26Cd52}Il2rg^{em26Cd22}/NjuCrl (NCG) from the Charles River Laboratory. On day 4 post-engraftment, mice were randomized to treatment arms. Mice received the following drugs and doses: weekly intraperitoneal injections of 10 mg/kg dinaciclib (MedChemExpress HY-10492) in 20% cyclodextrin (CTD THPB-P) diluted just prior to injection, daily oral gavage of 30 mg/kg gilteritinib (MedChemExpress HY-1432) in 6% drug w/w gelucire 44/14 (Gattefosse, France) aliquoted and mixed with drug weekly, oral gavage of vehicle control (gelucire 44/14), or combination therapy at single-agent regimens.

### RNA-Seq and Bioinformatics Pipeline

RNA was extracted with RNAeasy mini kit (Qiagen). The quality of RNA was assessed with Agilent 2100 BioAnalyzer and RNA 6000 Nano Kit and the amount was quantified with Qubit RNA HS Assay Kit. RNA-seq libraries were generated in triplicates per treatment/biological group. The RNA libraries were generated using the NEBNext® Ultra™ II Directional (stranded) RNA Library Prep Kit for Illumina (NEB #E7760L) and the NEBNext Poly (A) mRNA Magnetic Isolation Module (NEB #E7490) with the NEBNext Multiplex Oligos for Illumina Unique Dual Index Primer Pairs (NEB #6442S/L) using an input amount of 200 ng total RNA (quantified using Qubit Fluorometer) according to manufacturer's protocol.

### Results

Genome-Wide CRISPR Screen Reveals Novel Synthetic Lethal Partners with Gilteritinib in FLT3-ITD Cells

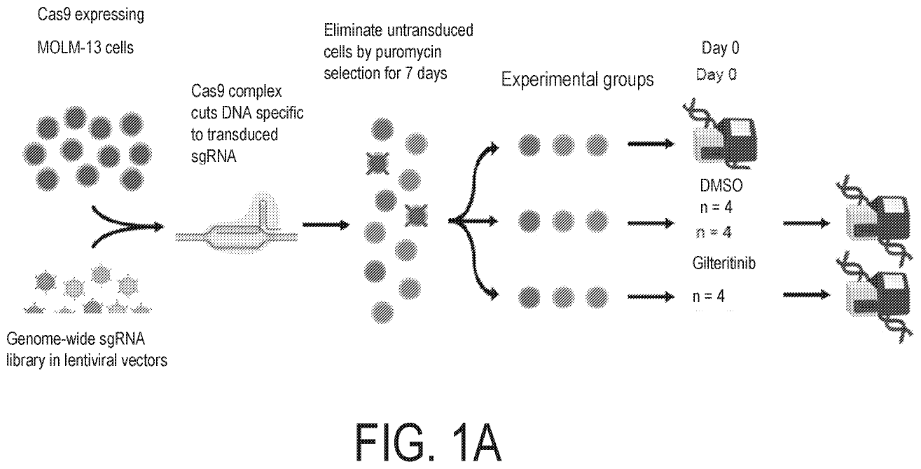

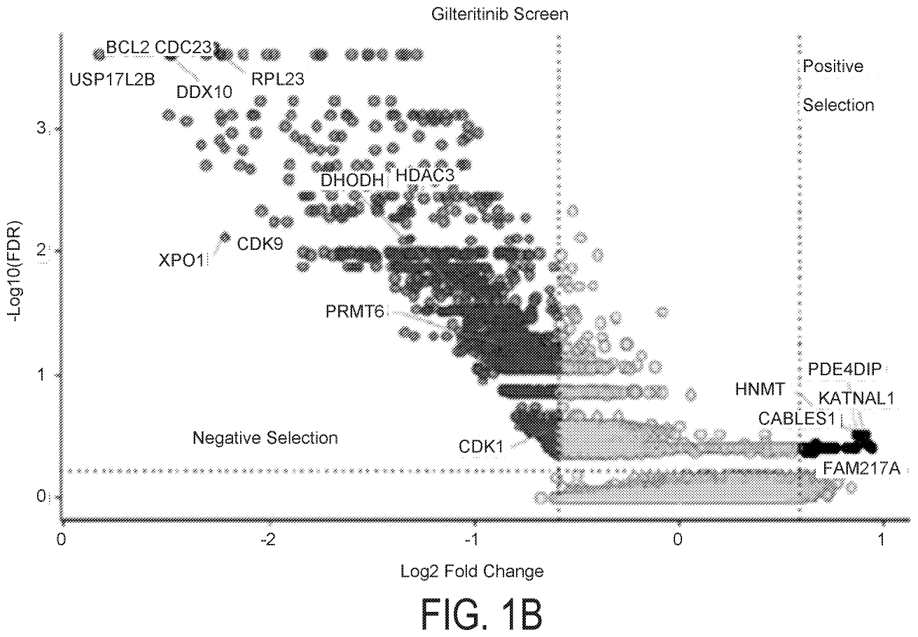

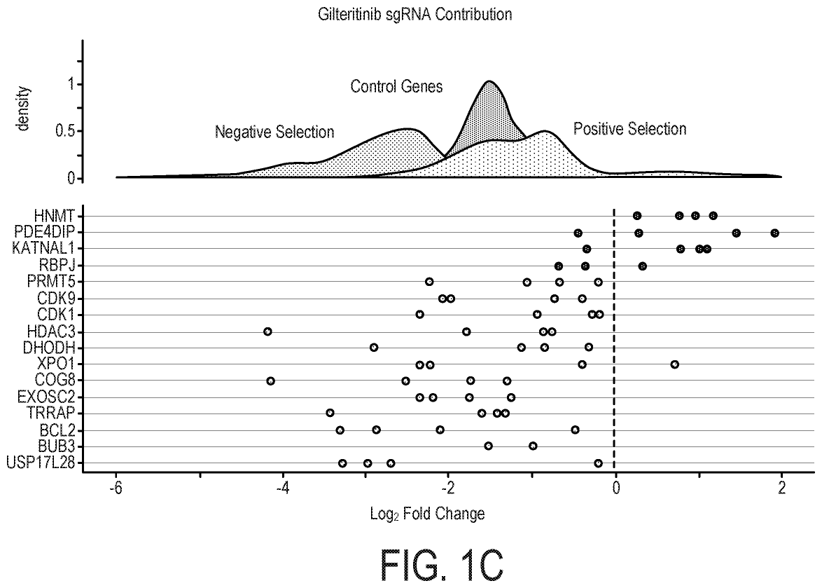

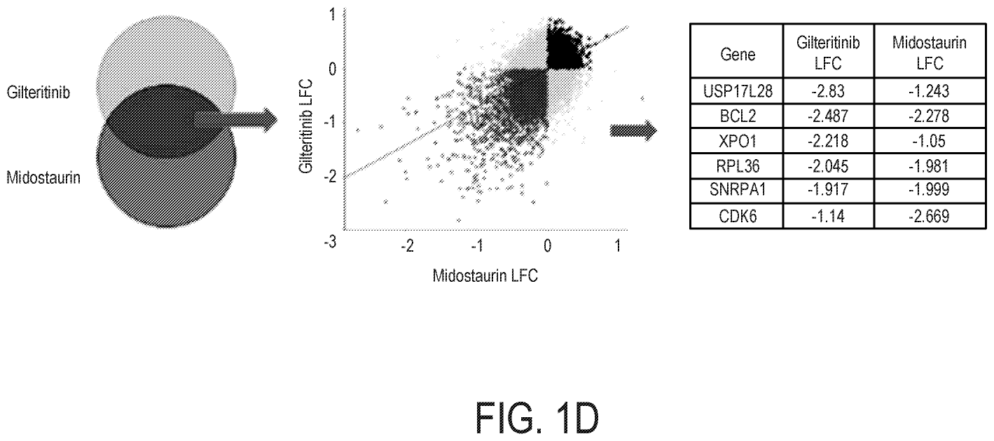

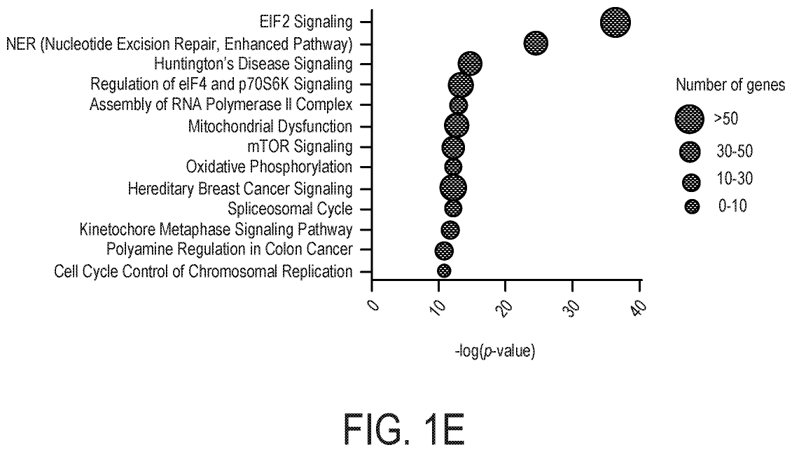

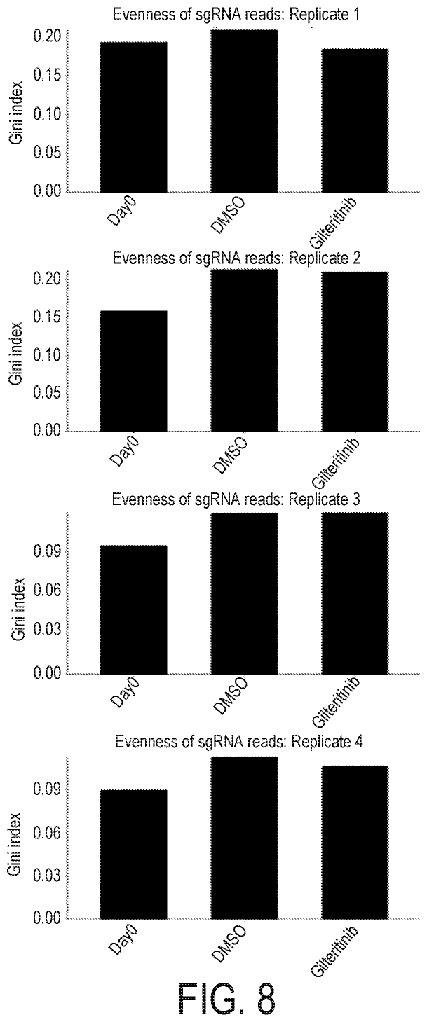

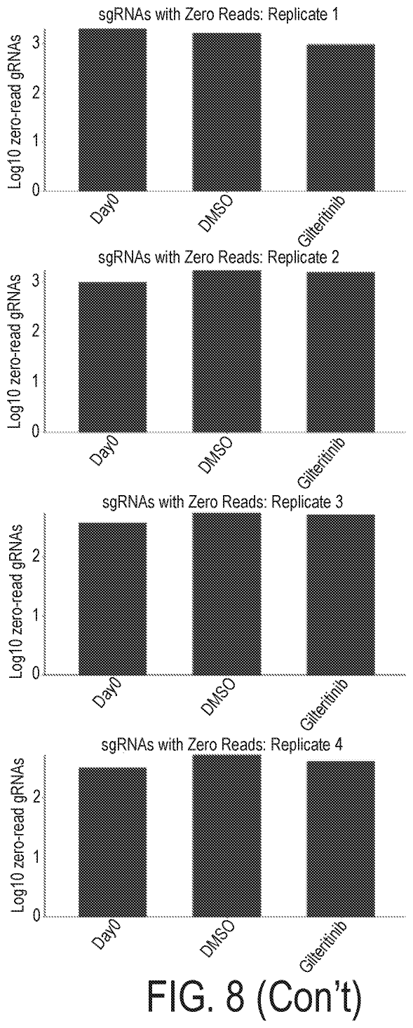

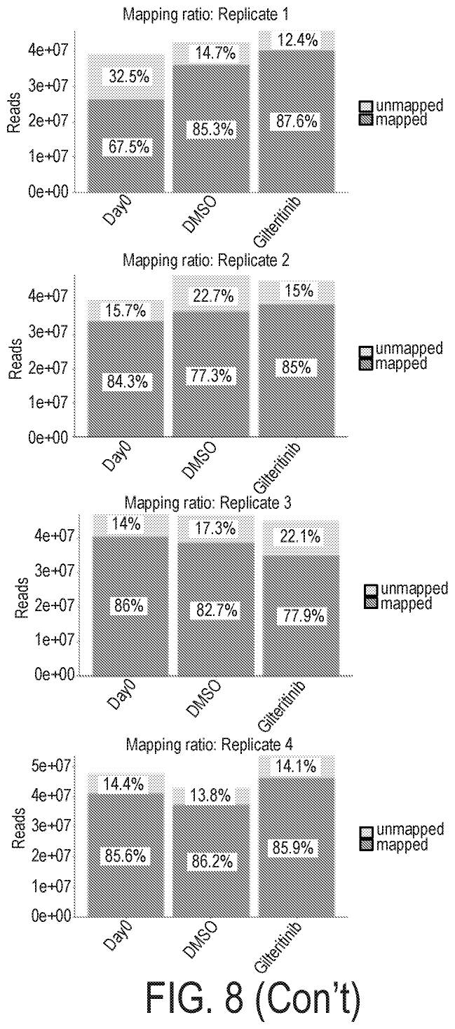

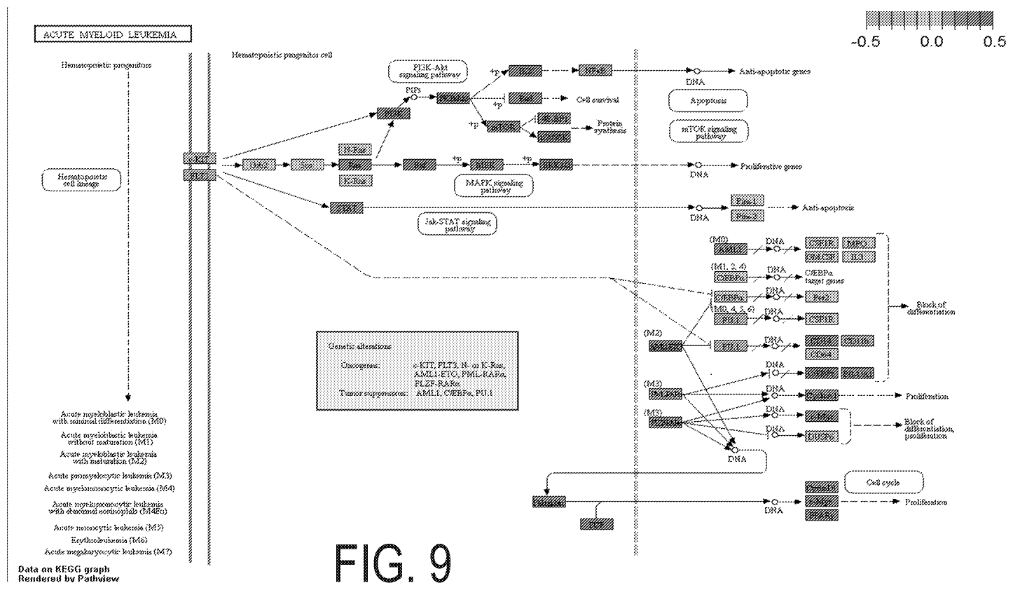

To identify co-essential genes and pathways that sensitize FLT3-ITD AML cells to gilteritenib, a genome-wide CRISPR screening was conducted on MOLM-13 cells (FIG. 1A). Potential targets were highlighted based upon statistically significant thresholds for negative selection (synergistic) and positive selection (antagonistic) following a robust rank aggregation (RRA) analysis (FIG. 1B)⁸. Across all four replicates, the screens demonstrated low Gini indices (a metric to account for heterogeneity of sgRNA reads), a low quantity of missed sgRNAs, and a small percentage of unmapped reads (FIG. 8)⁹. sgRNA targeting genes involved in cell cycle (CDK9 and CDK1), metabolism (DHODH), and epigenetic regulation (HDAC3 and PRMT5) were among top depleted hits upon gilteritinib treatment. These hits show the highest degrees of consistency of dropout, implying strong synergistic interactions with gilteritinib (FIG. 1C) without being previously identified by our midostaurin screen^{5,6}. BCL2 and XPO1 also stood out among top ranked hits as synthetic lethal targets (FIG. 1D)^{15,16}. KEGG pathway analysis revealed that perturbation of multiple genes in FLT3 and cKIT signaling pathways confers vulnerability to FLT3 inhibitor (FIG. 9). Ingenuity Pathways Analysis (IPA) pathway analysis focusing on sgRNAs that were depleted by at least 3.9-fold (LFC<−0.6) showed that most of their targeted genes were significantly enriched in AML-related biological processes that regulate translation initiation, mitochondria dysfunction, OXPHOS, kinetochore metaphase signaling and cell cycle control of chromosomal replication (FIG. 1E).

shRNA Knockdown Validates Screen Predictions of Selected Genes as Synthetic Lethal Targets for FLT3 Inhibitor

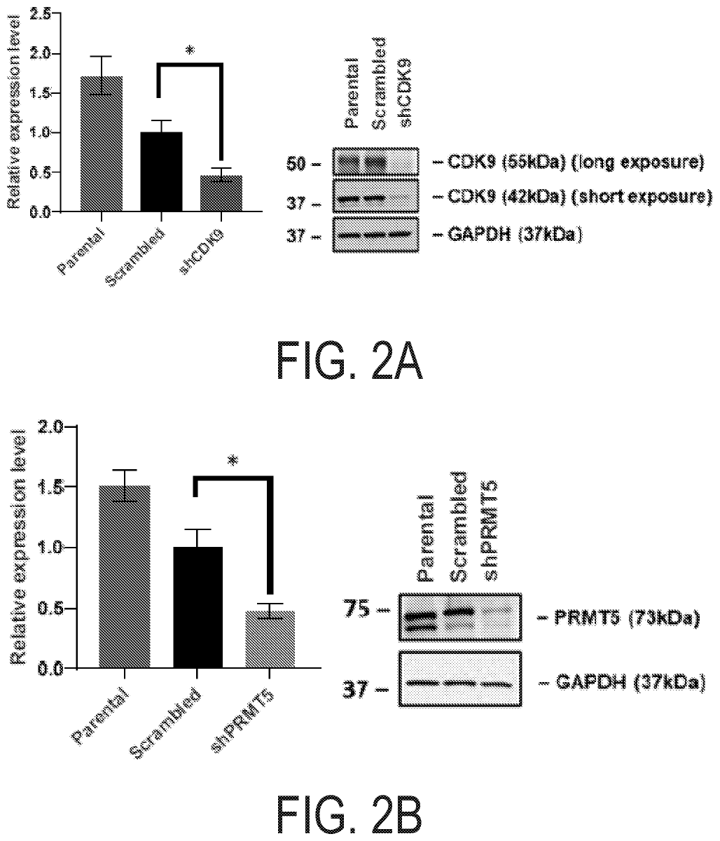

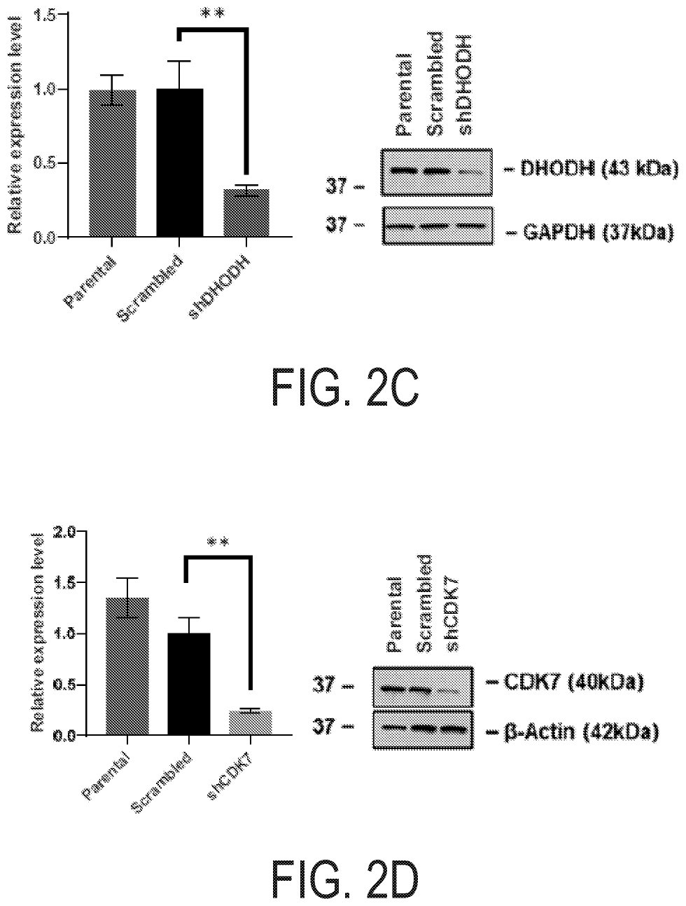

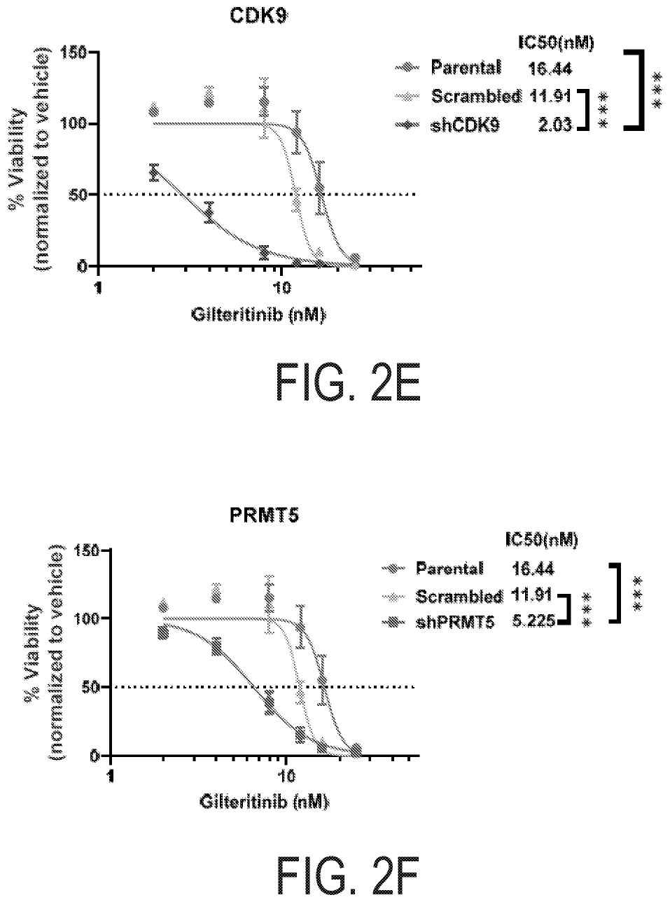

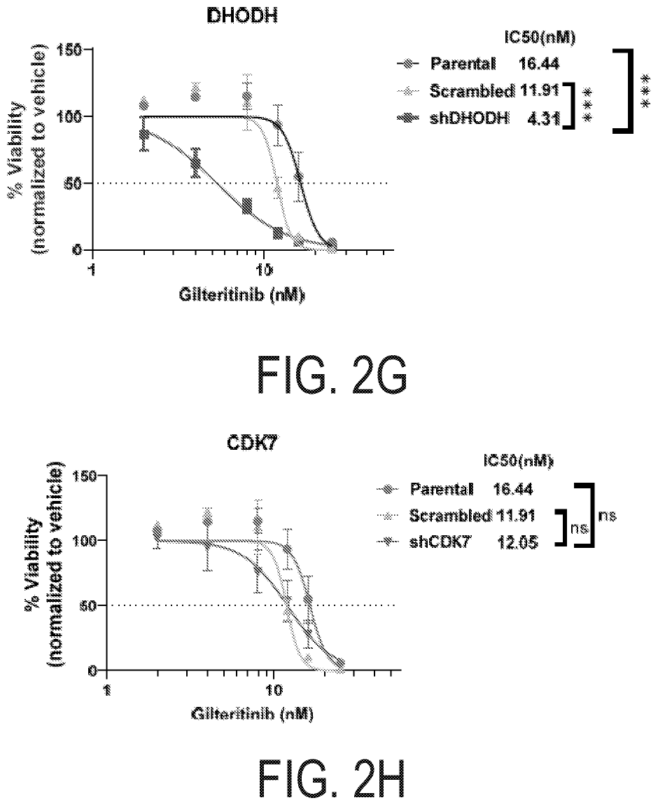

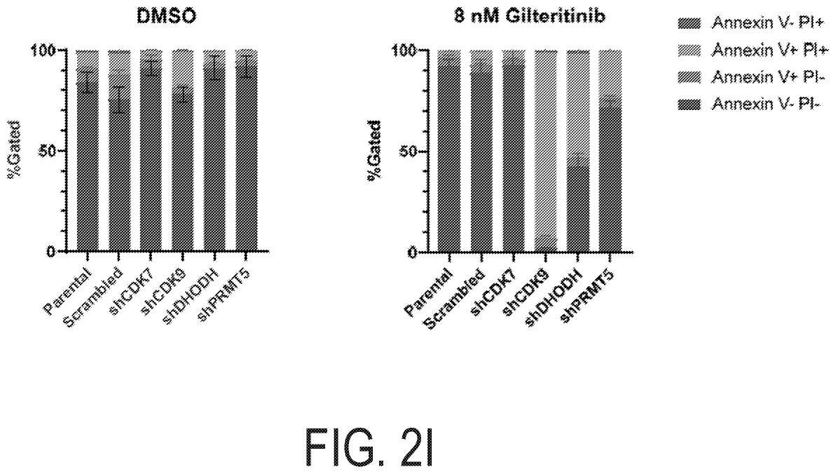

DHODH, CDK9 and PRMT5 genes were selected for validation, in light of their strong synergistic interactions with gilteritinib and availability of clinical grade inhibitors. MOLM-13 cells were transduced with shRNA targeting each of these genes and knockdown efficiencies were confirmed via qPCR and Western Blotting (FIG. 2A-D). Scrambled or gene-targeting shRNA transduced cells were treated with gilteritinib (2-25 nM dose range) for 120 hours followed by MTS analysis to quantify cell proliferation. Cells with knockdown of CDK9, DHODH or PRMT5 had a remarkably lower IC₅₀ of gilteritinib compared with scrambled or the parental (untransduced) controls (FIG. 2E-G). In contrast, knockdown of CDK7, a gene expected to lack synergistic interaction with gilteritinib according to CRISPR screen results, only had a marginal impact on cell viability compared to scramble control (FIG. 2H). In agreement with the proliferation assay, knockdowns of CDK9, DHODH, and PRMT5 substantially increased the percentages of necroptotic/apoptotic cells by 9-, 5- and 2.6-fold, respectively, compared to scrambled control cells in response to gilteritinib treatment (FIG. 2I). Conversely, knockdown of CDK7 did not produce the same biological effect in the presence of gilteritinib, supporting the prediction that inactivation of CDK9, DHODH, or PRMT5 could sensitize AML cells to FLT3-ITD inhibitors.

shCDK9 or shDHODH-Mediated Synthetic Lethality Rewires the Transcriptional Programs of Gilteritinib-Treated AML

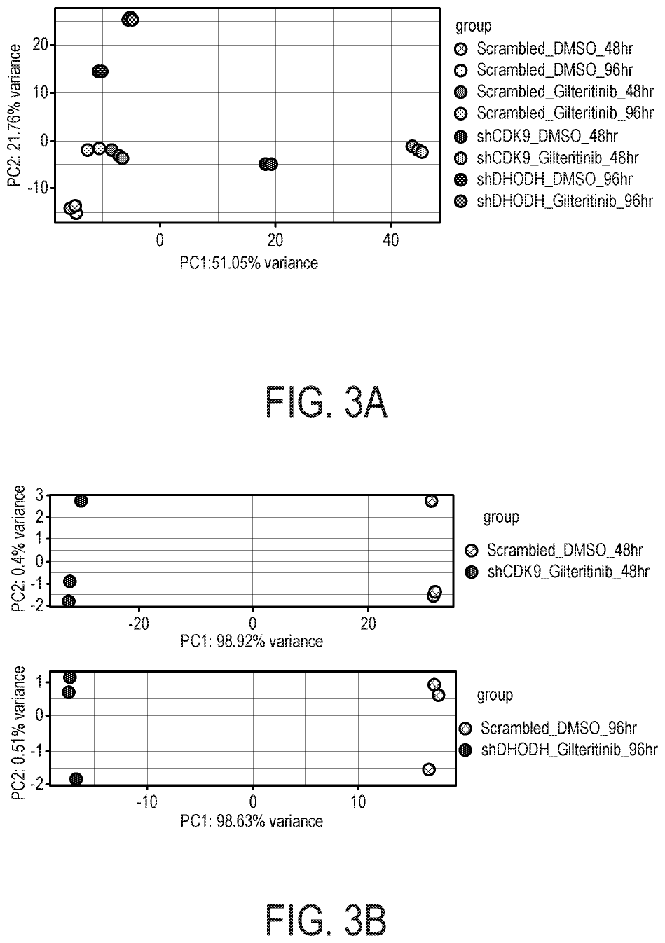

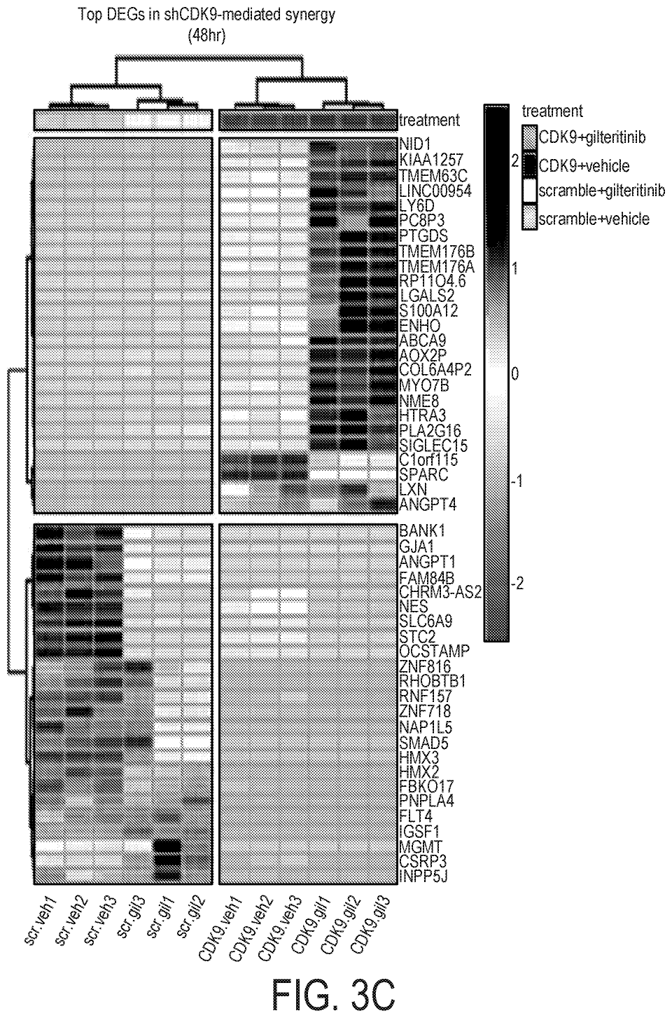

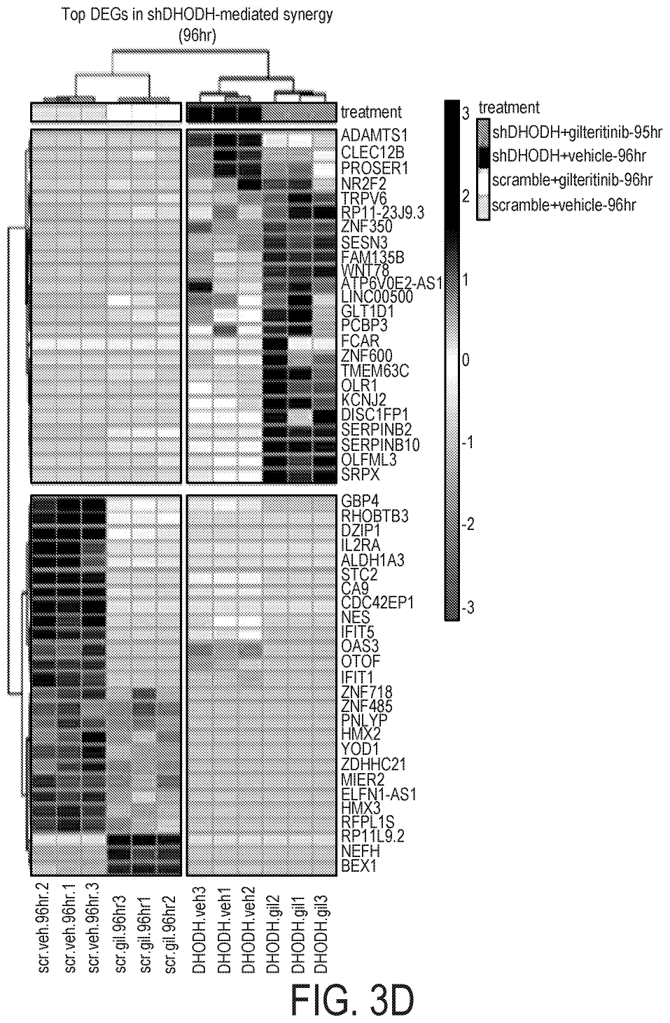

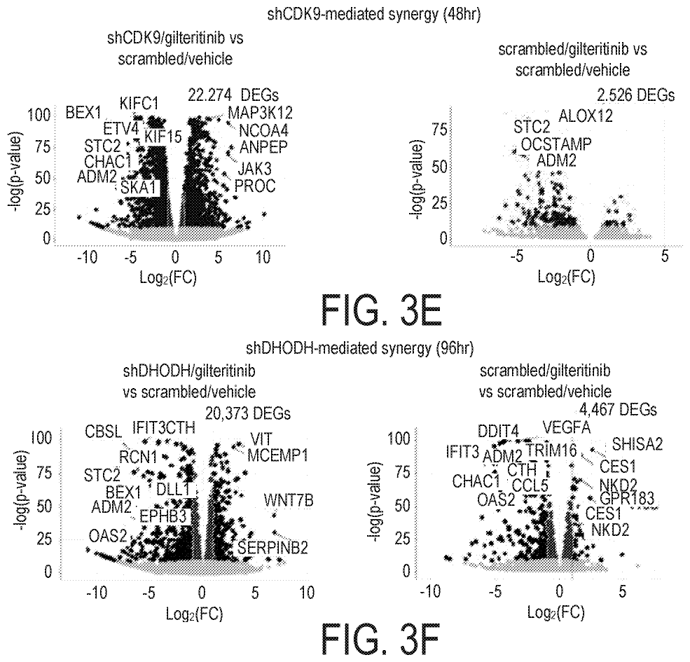

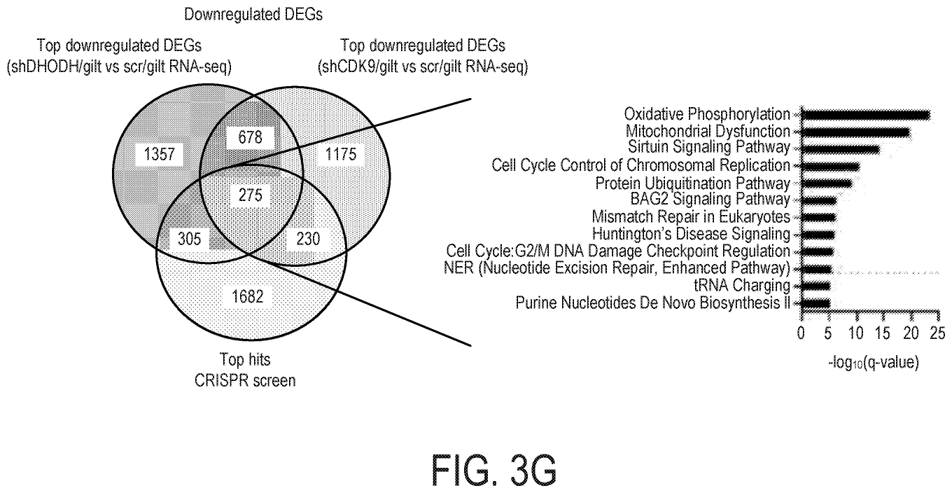

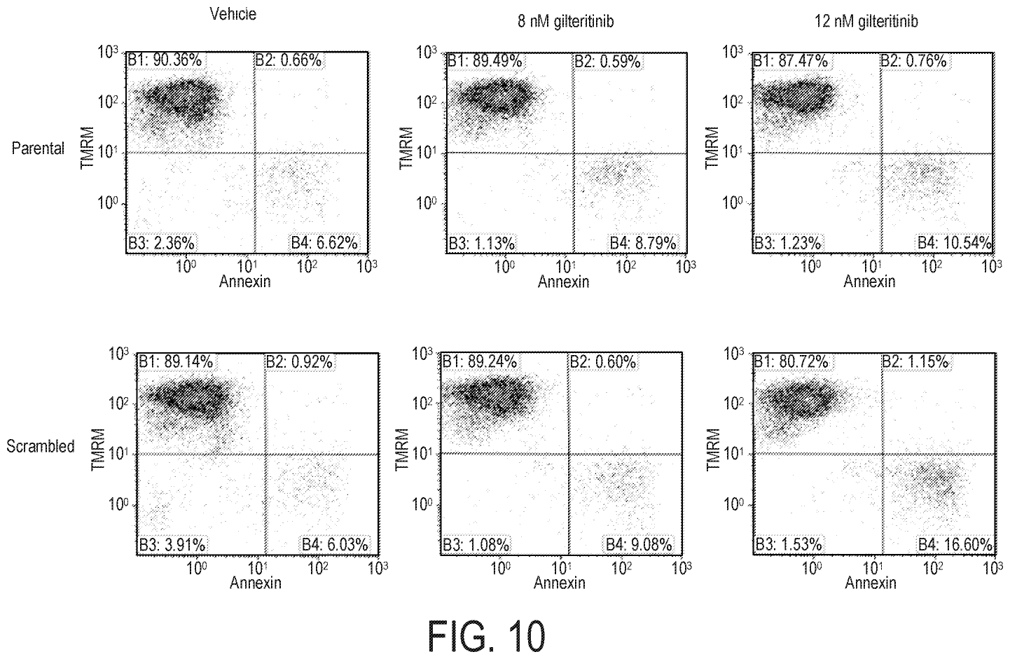

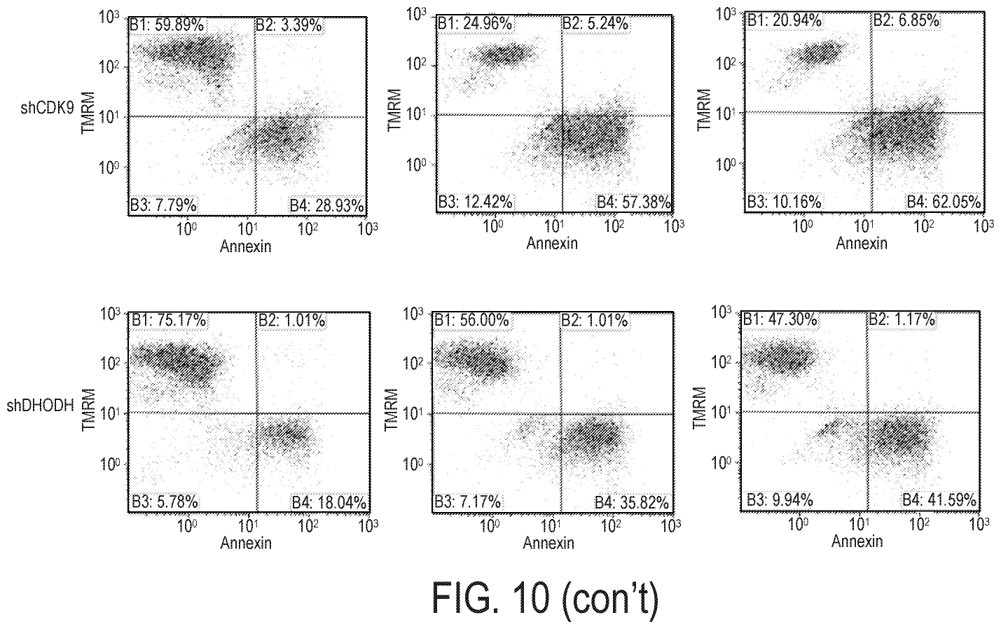

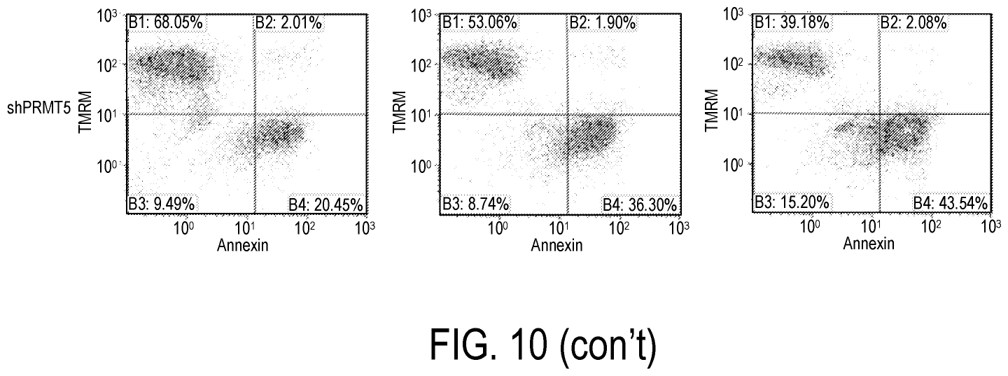

To dissect the underlying mechanism of synergy, RNA-seq was performed on scrambled, shCDK9, and shDHODH MOLM-13 cells treated with a sublethal concentration of gilteritinib (8 nM, FIG. 10) for 48 or 96 hr. After removal of the low abundant genes and normalization, principal component analysis (PCA) plots highlighted distinctive transcriptional features of the combination treatments with respect to single agent-treated samples (FIG. 3A-B). Based on heatmaps derived from hierarchical clustering, the transcriptional patterns of top differentially expressed genes (DEGs) in shCDK9/gilteritinib and shDHODH/gilteritinib combination groups showed stark differences in expression directionality when compared to their respective controls (FIG. 3C-D). As depicted by volcano plots, 48 and 96-hr gilteritinib treatment alone yielded 2, 526 DEGs and 4,467 DEGs, respectively (FIG. 3E-F, right panel). shCDK9/gilteritinib and shDHODH/gilteritinib combinations increased the numbers of DEGs to 22, 274 and 20, 373, respectively (FIG. 3E-F, left panel). In comparison to shCDK9, shDHODH, or gilteritinib treatment alone, shCDK9/gilteritinib or shDHODH/gilteritinib combined treatment also differentially modulated the expressions of a number of genes, showing DHODH or CDK9 knockdown orchestrates drastic transcriptome alterations in gilteritinib-treated AML cells.

An overlay of top depleted hits from the CRISPR screen and top downregulated DEGs from RNA-seq is shown in FIG. 3G. 810 top hits from CRISPR screen negative selection were also differentially downregulated in shCDK9/gilteritinib (CDK9 knockdown effect; 580 including 305 and 275 genes) or shDHODH/gilteritinib (DHODH knockdown effect; 505 including 230 and 275 genes) combination data sets, respectively, while 275 genes were shared by all three data sets. Pathway analysis of the 275 overlapping genes demonstrates alterations in OXPHOS (SDHA, UQCRCI, and UQCRQ), cell cycle (CDC45 and CDC23), purine de novo biosynthesis (GMPS, GART and PAICS), mevalonate pathway (HMGCSI), and glycolysis (ALDOA and ENO1), showing that the synergistic effect of knockout of CDK9 or DHDOH and FLT3 inhibition is dependent on functional suppression of these pathways. Of note, PRMT5 transcripts were reduced in both shCDK9/gilteritinib and shDHODH/gilteritinib DEG data sets, implying that PRMT5 is transcriptionally located downstream of shCDK9/gilteritinib or shDHODH/gilteritinib treatments. Among all differentially upregulated genes, 187 were shared by shCDK9/gilteritinib RNA-seq and shDHODH/gilteritinib RNA-seq data sets (FIG. 3H). These shared DEGs were enriched in pyroptosis and phagosome formation pathways, implying that combination-treated cells undergo pyroptosis.

shCDK9 or shDHODH Confers Sensitivity to Gilteritinib Treatment by Downregulating Metabolic and Proliferation Pathways

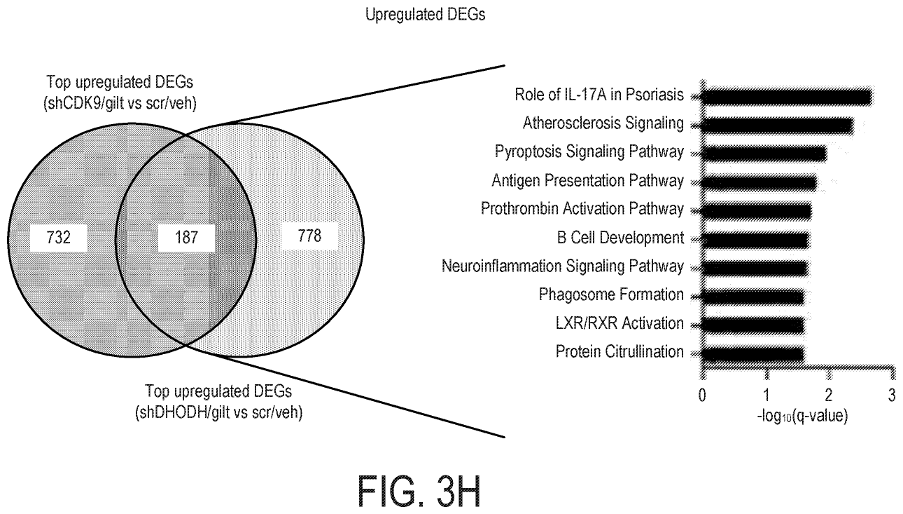

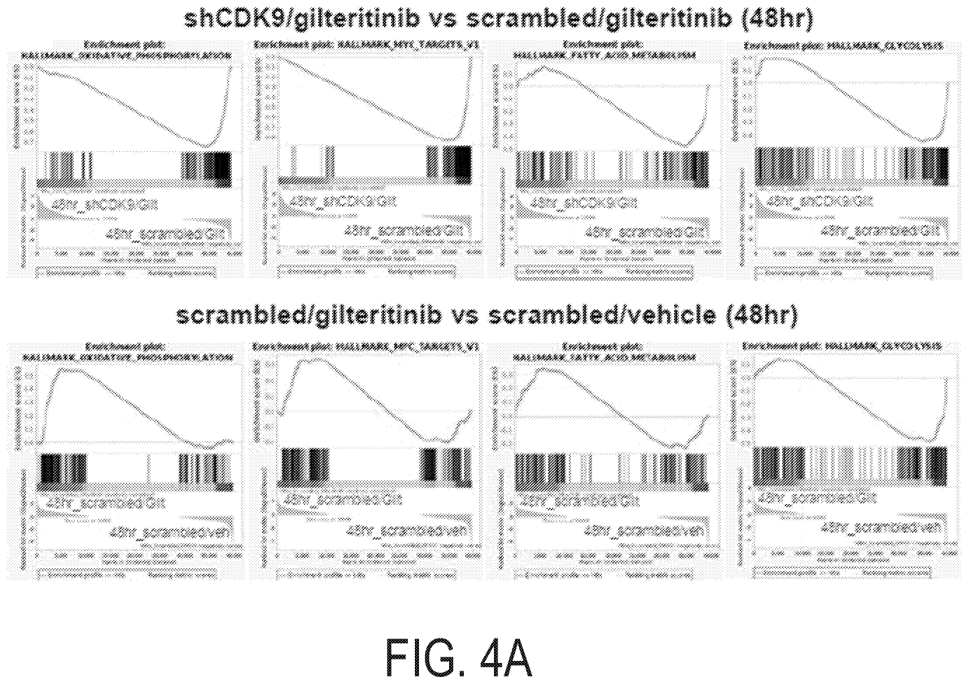

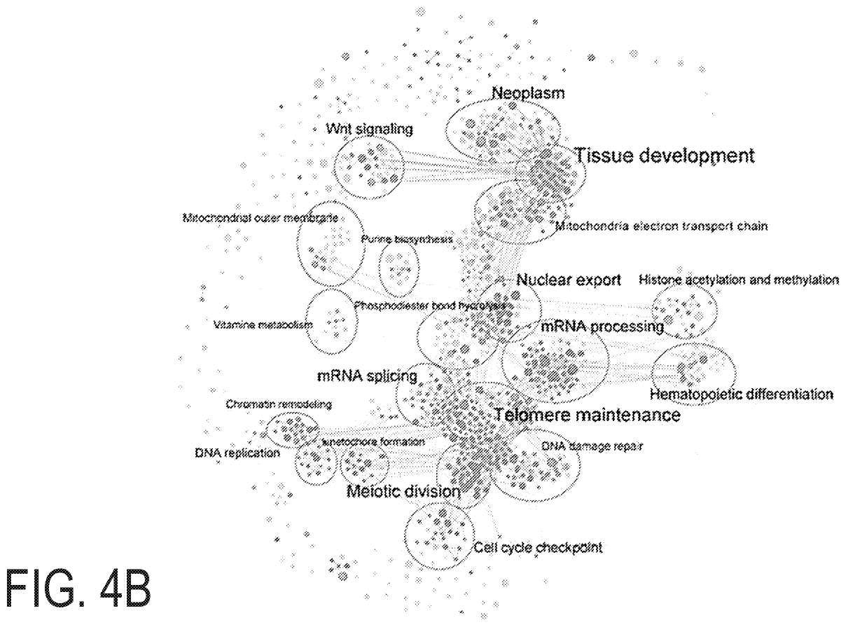

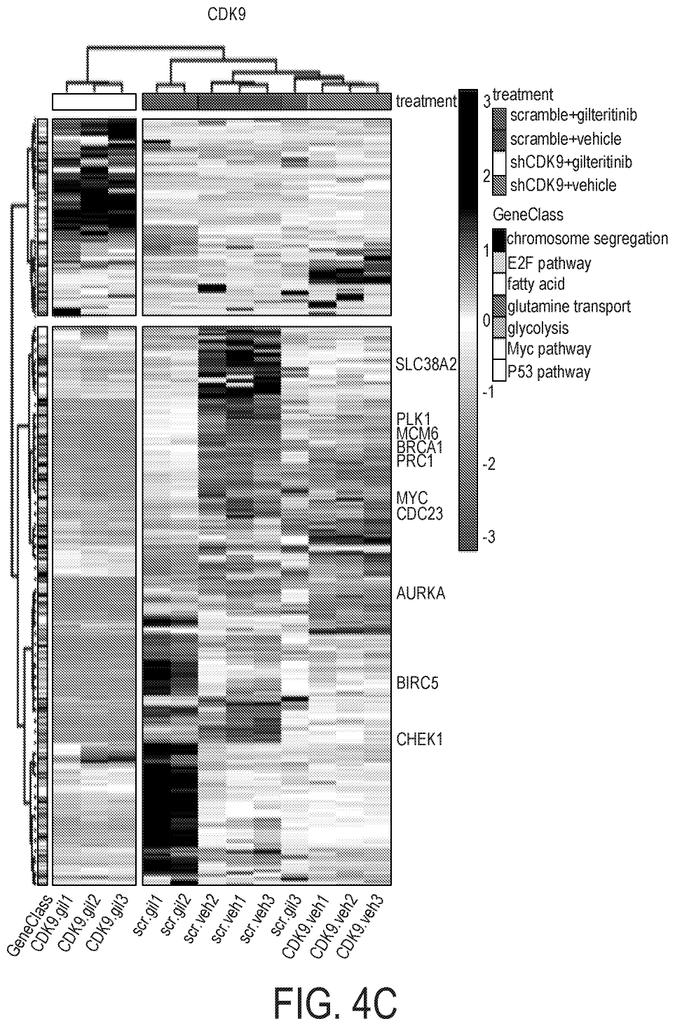

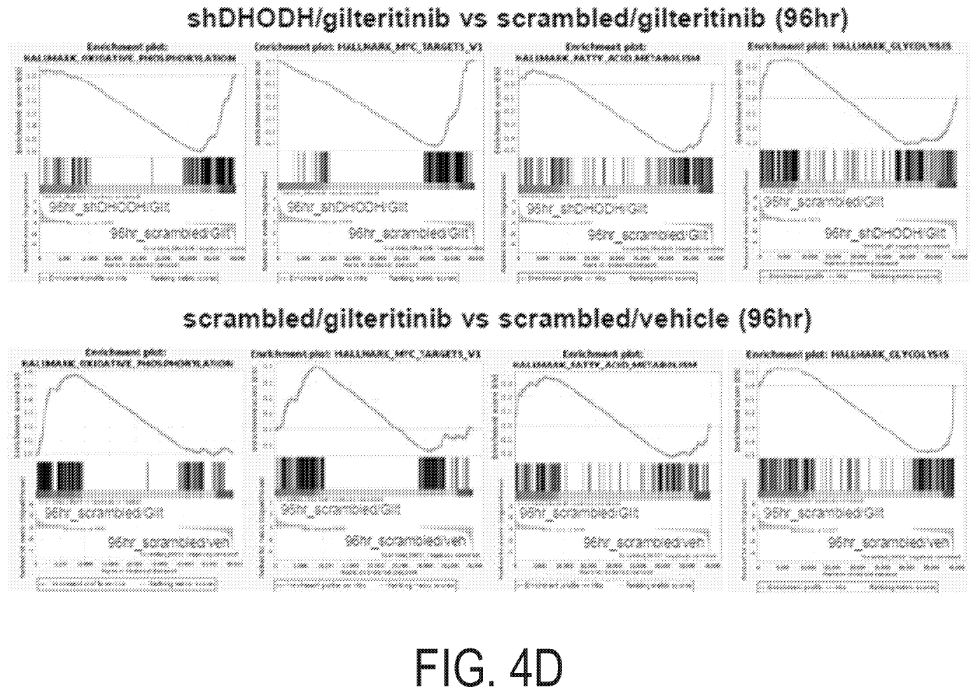

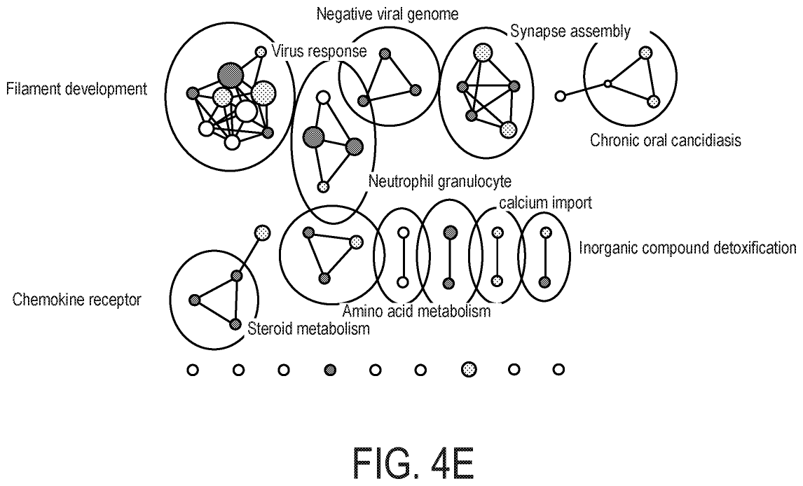

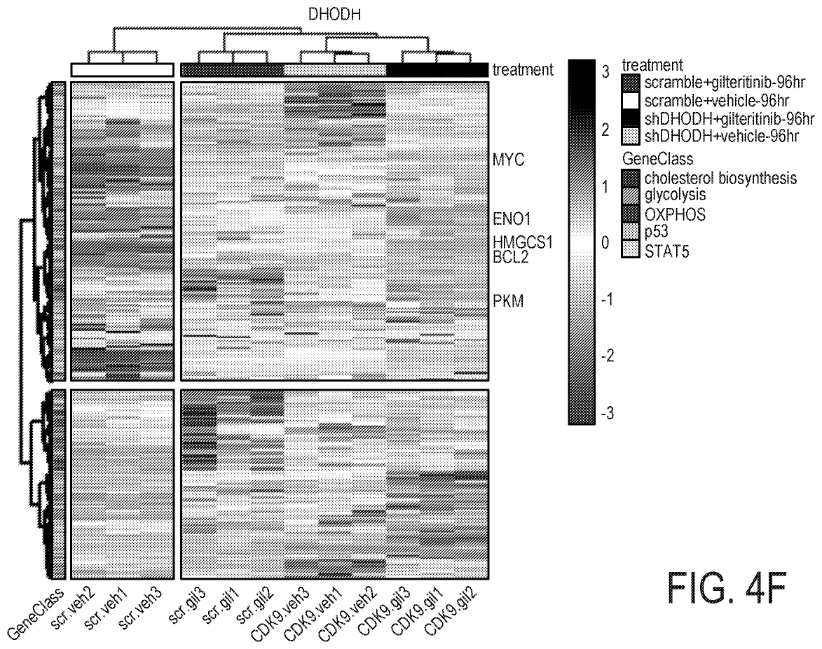

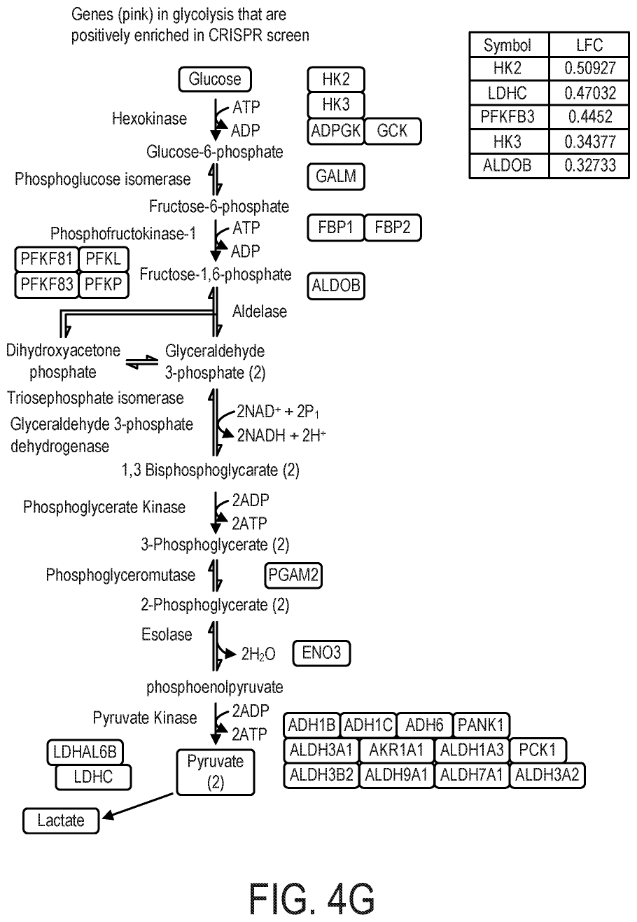

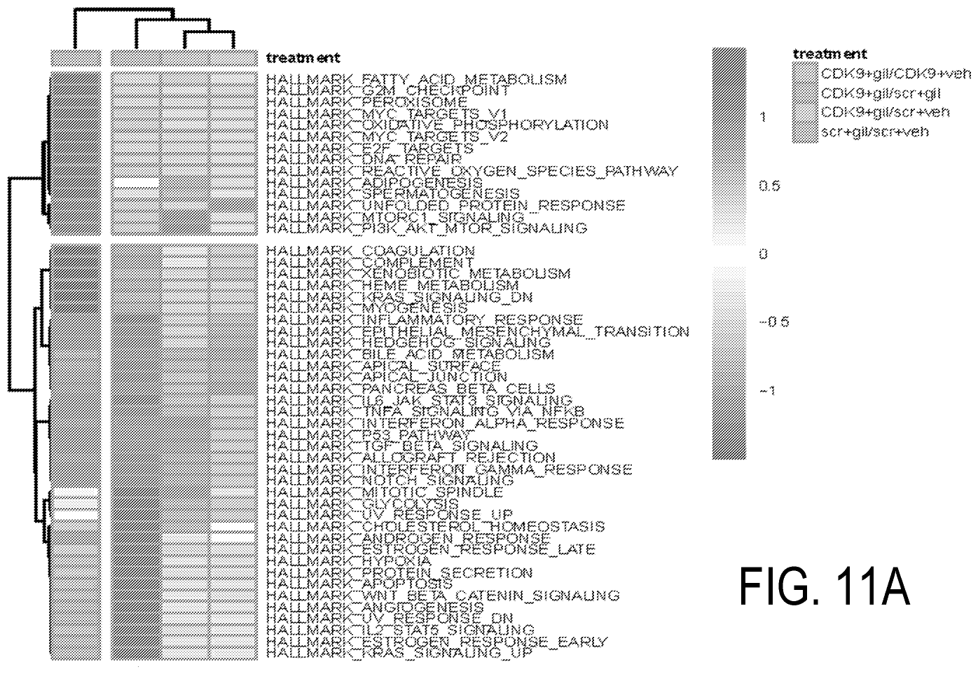

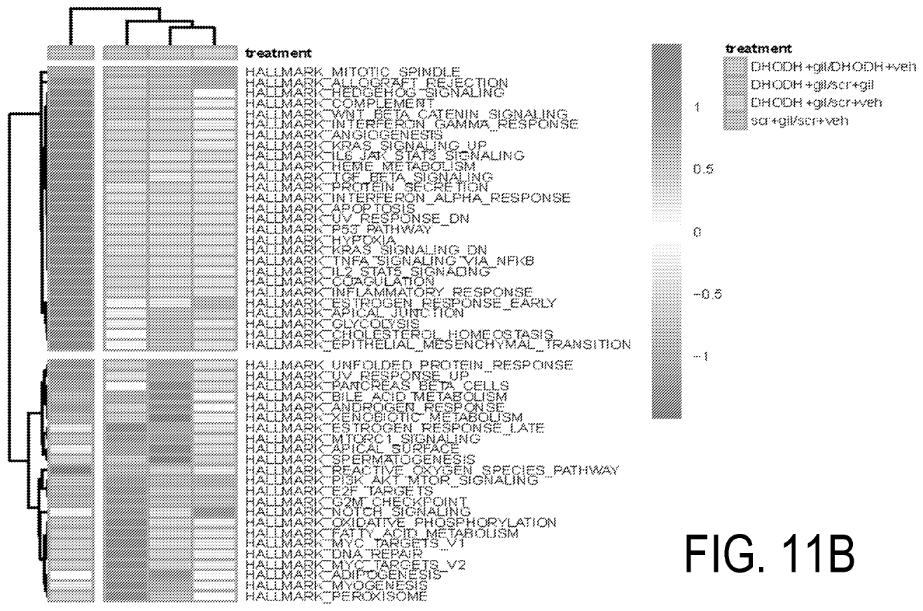

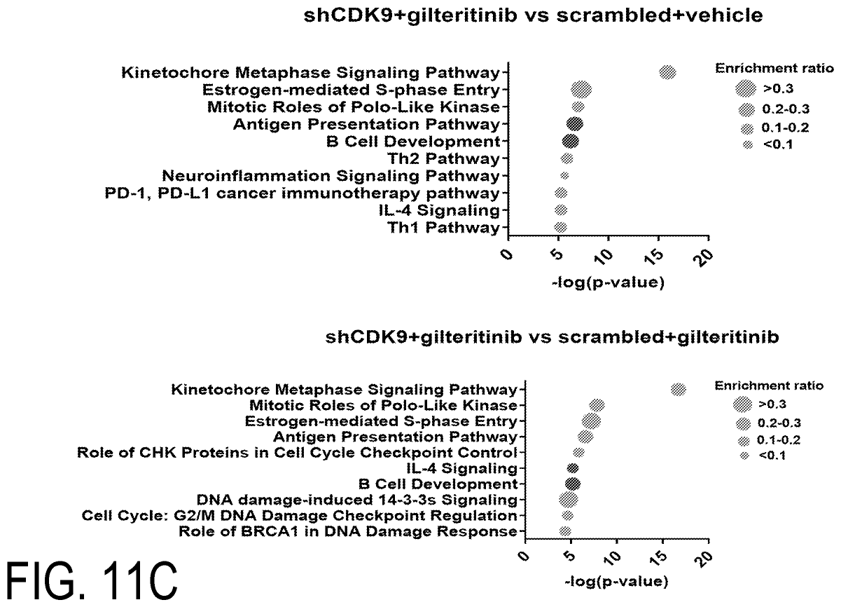

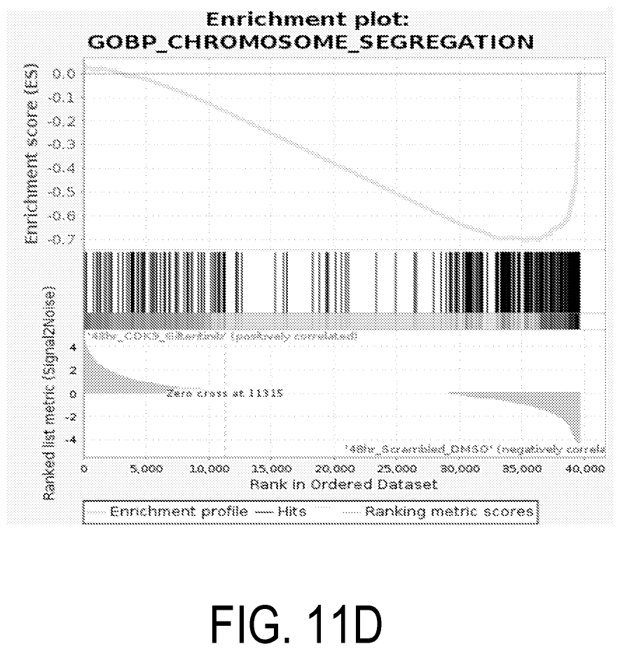

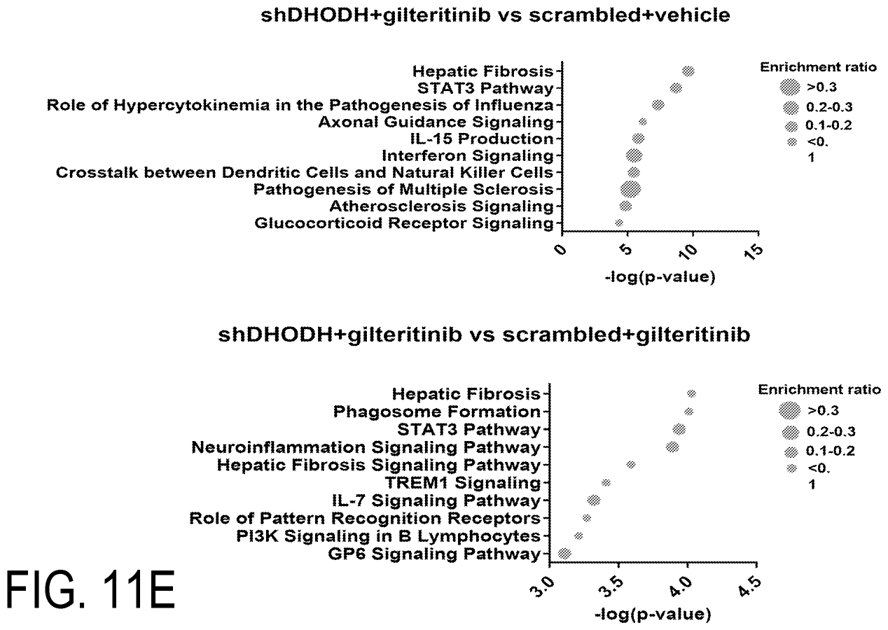

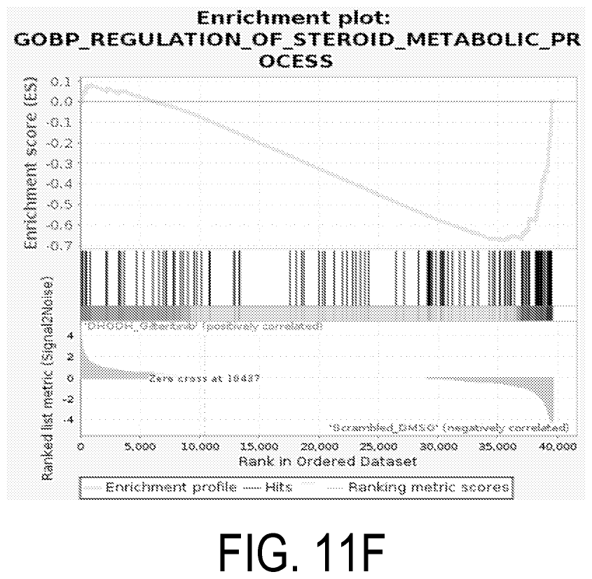

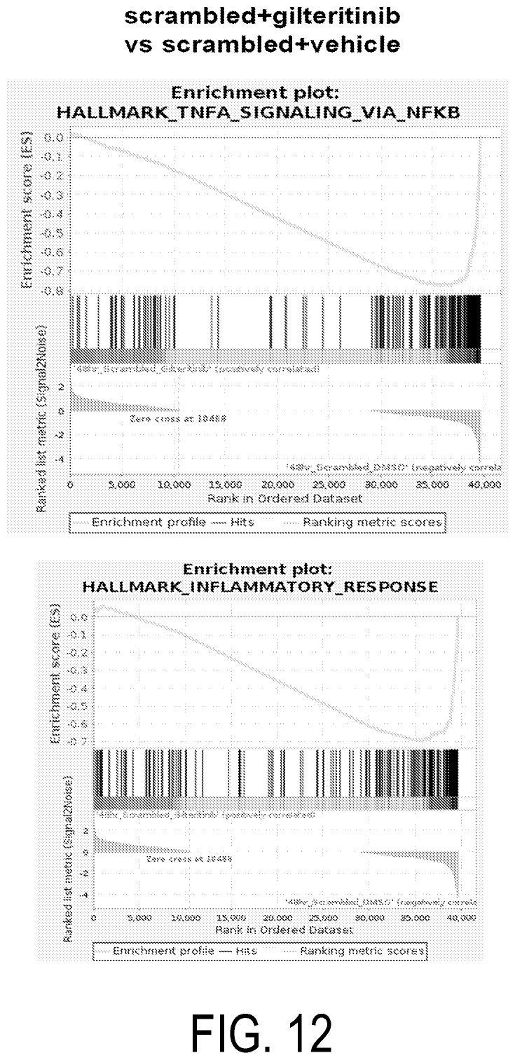

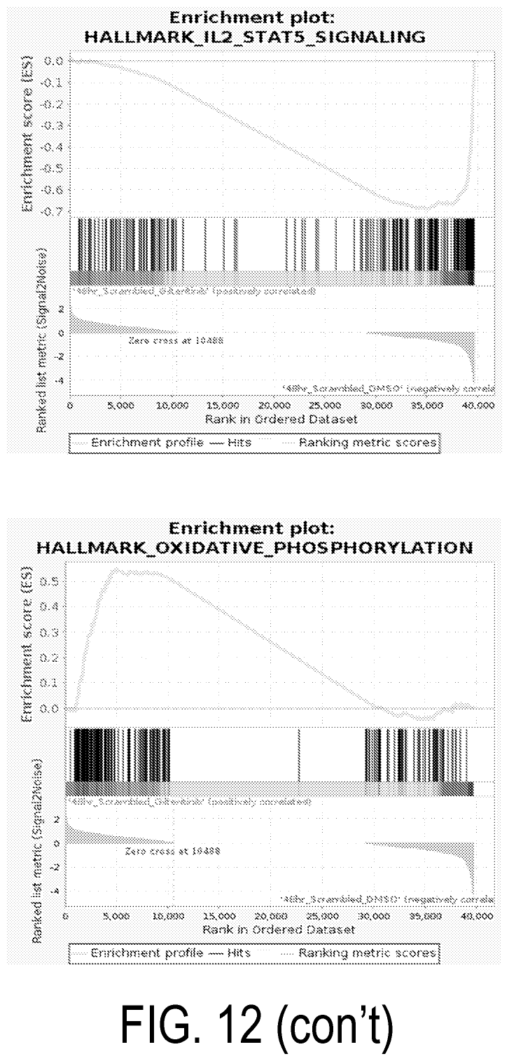

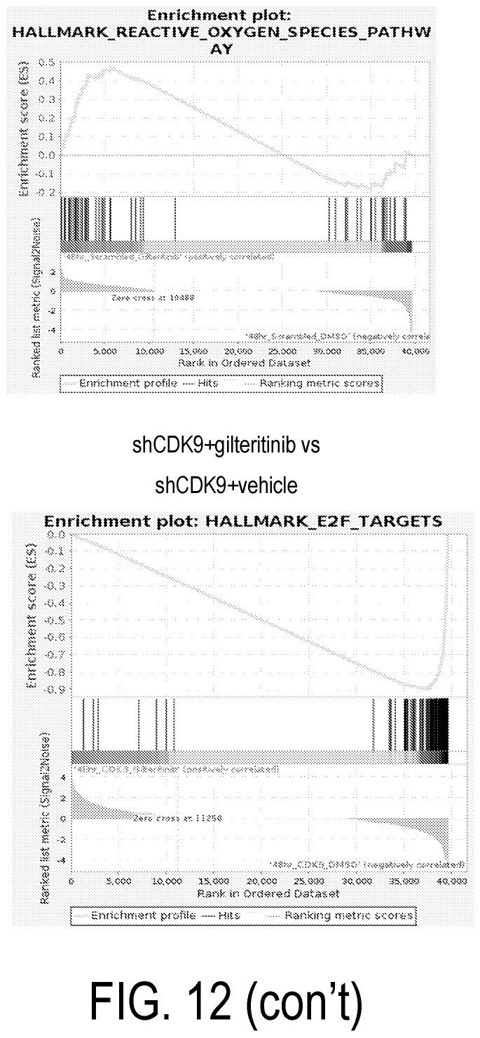

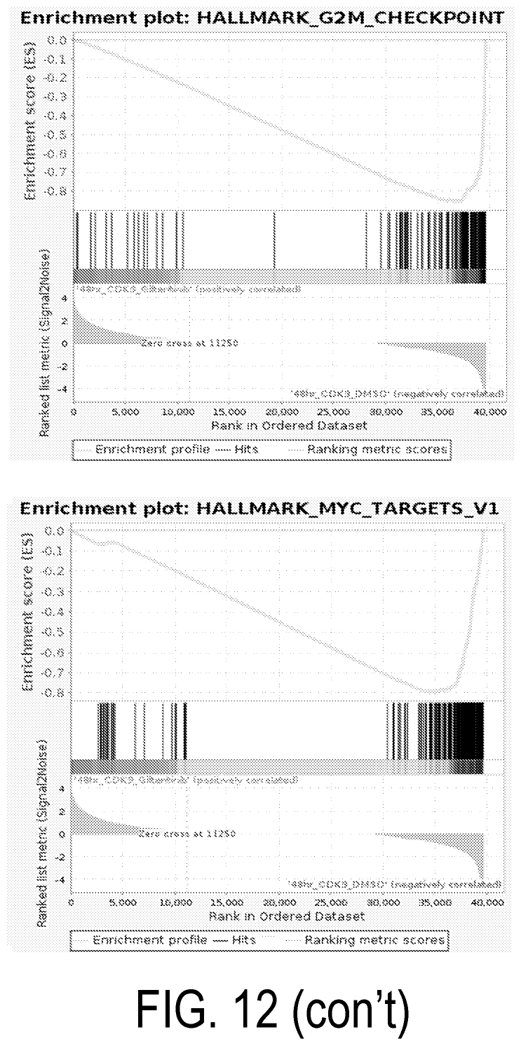

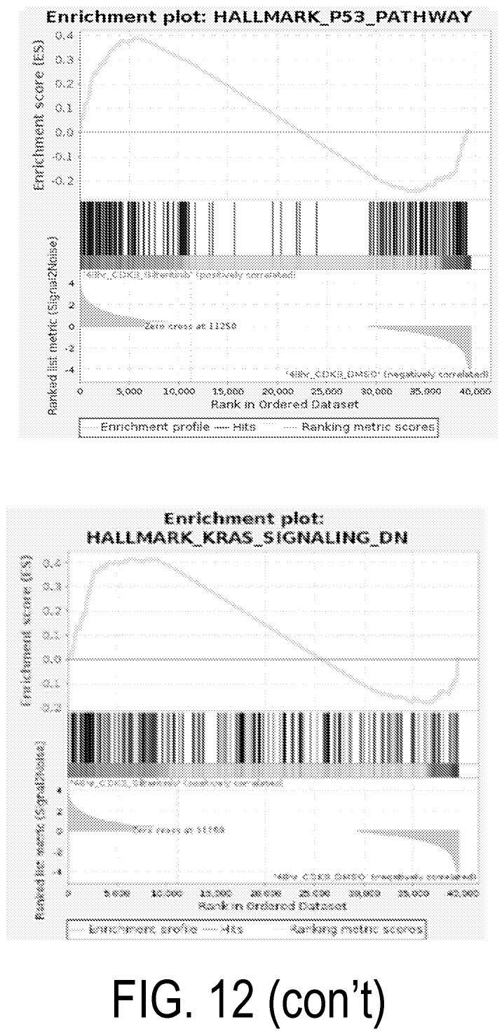

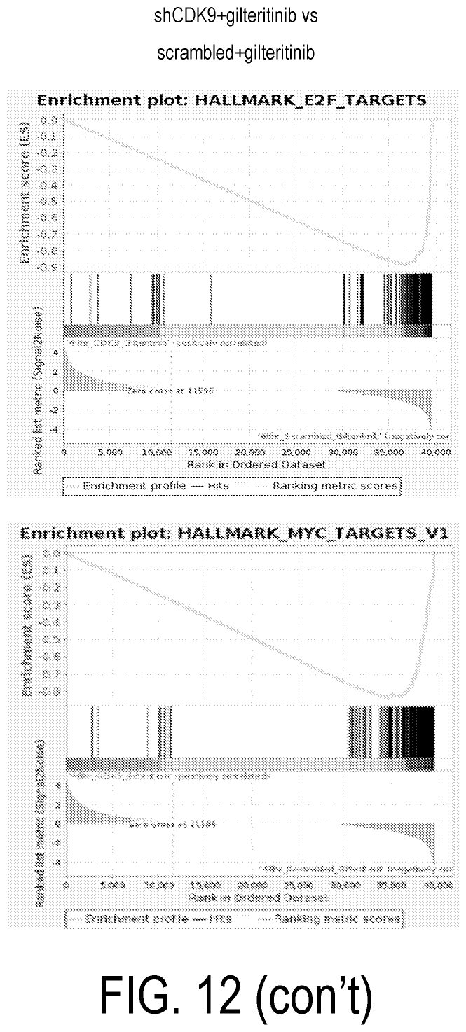

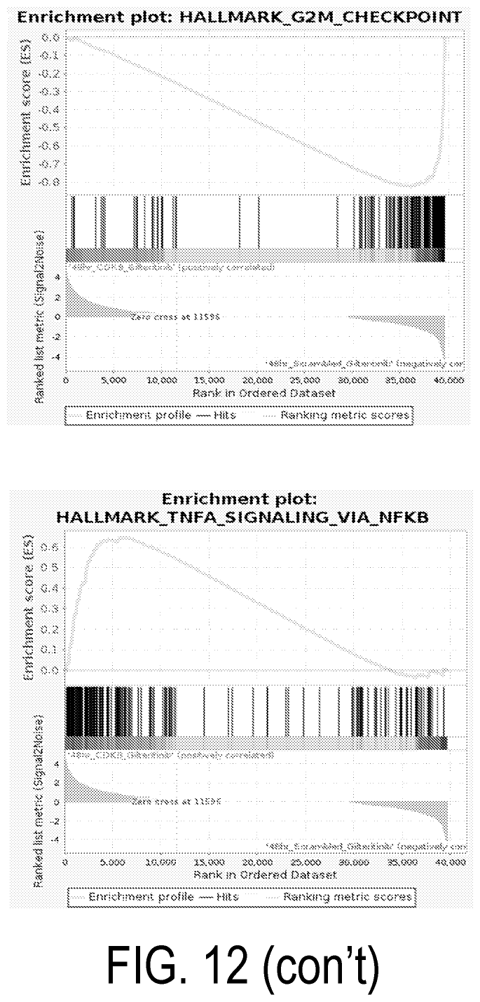

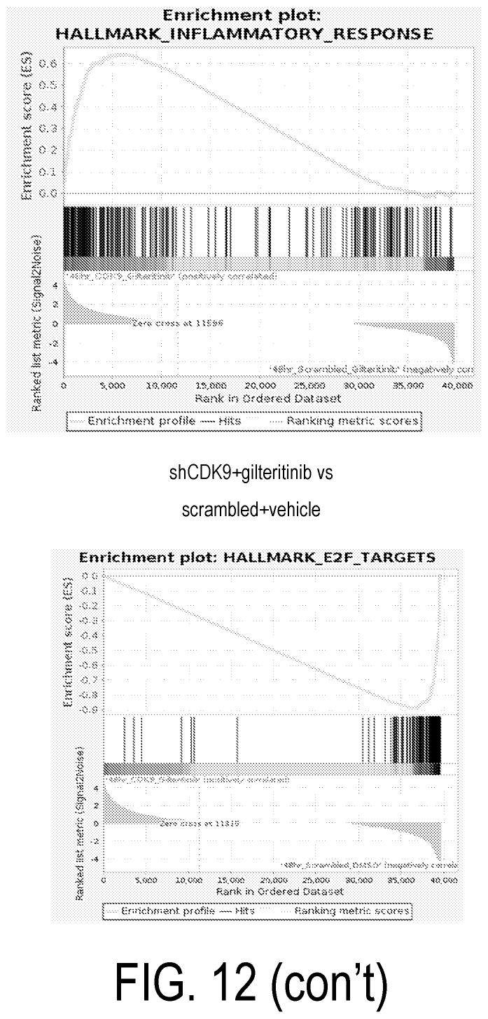

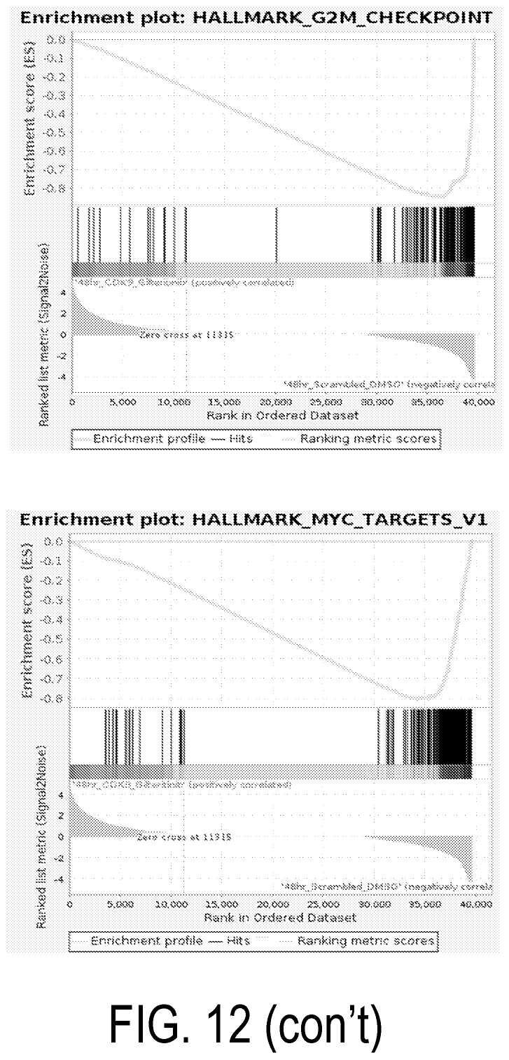

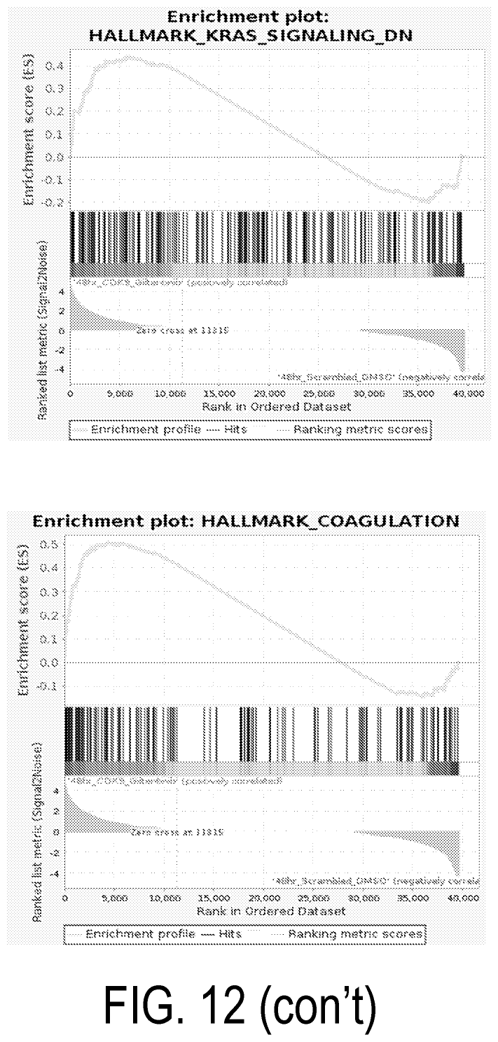

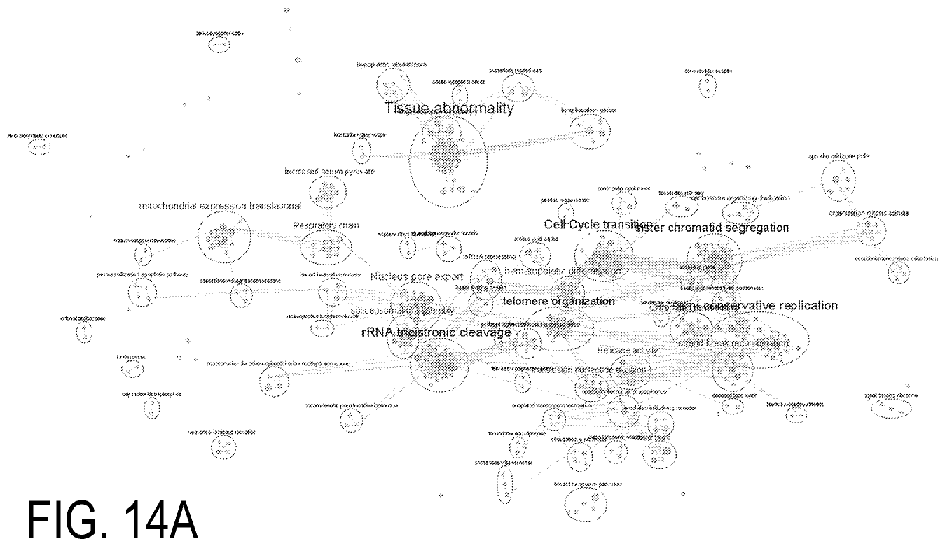

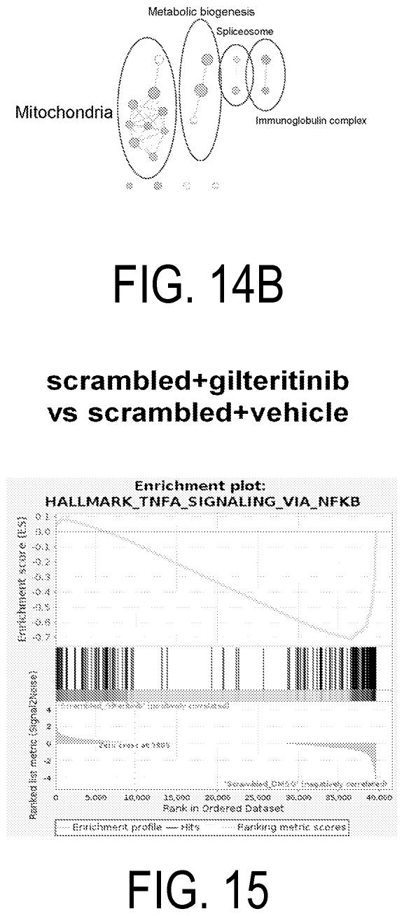

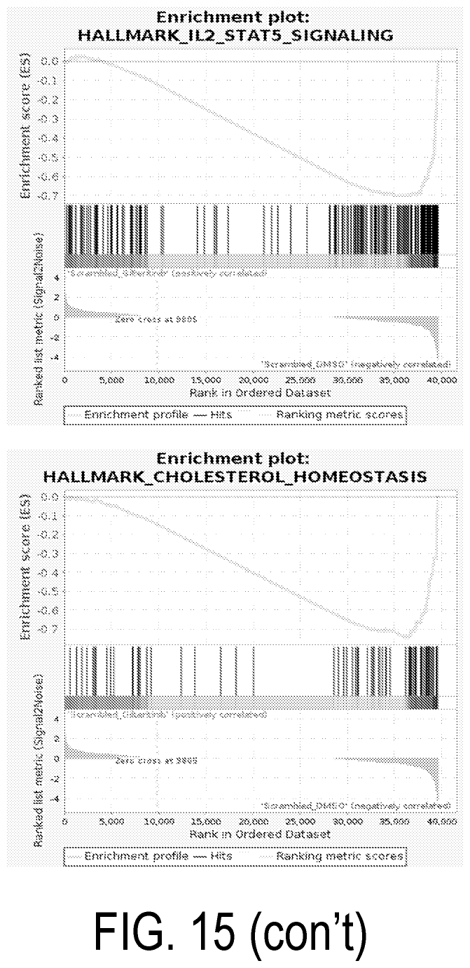

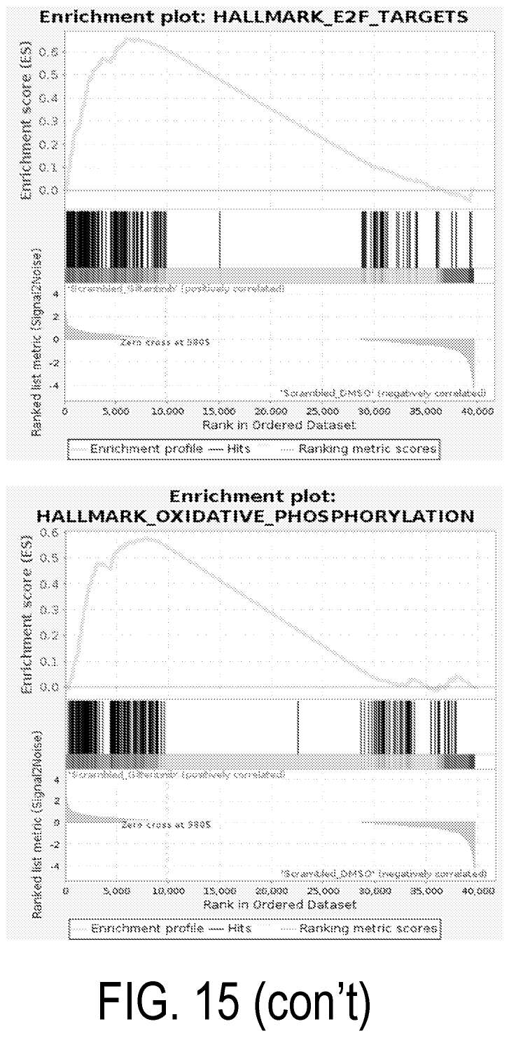

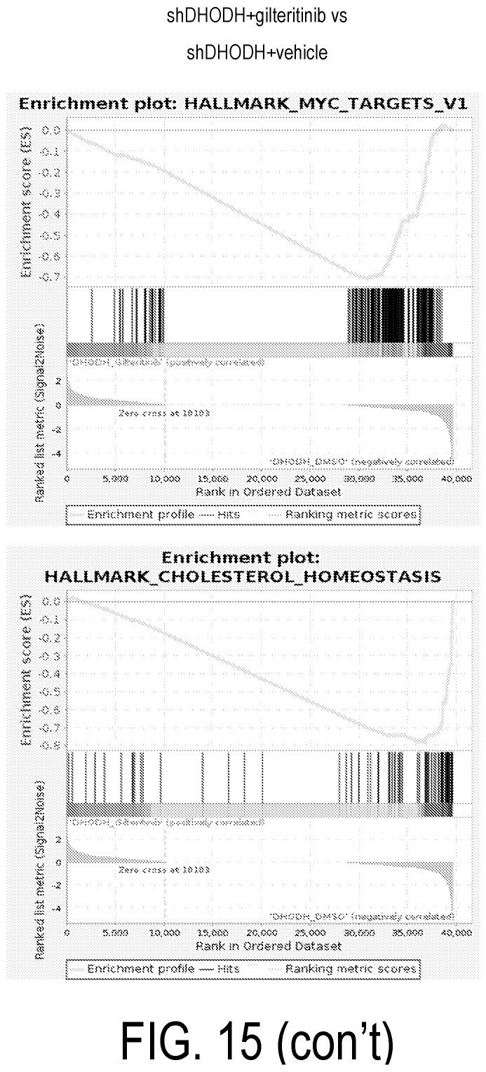

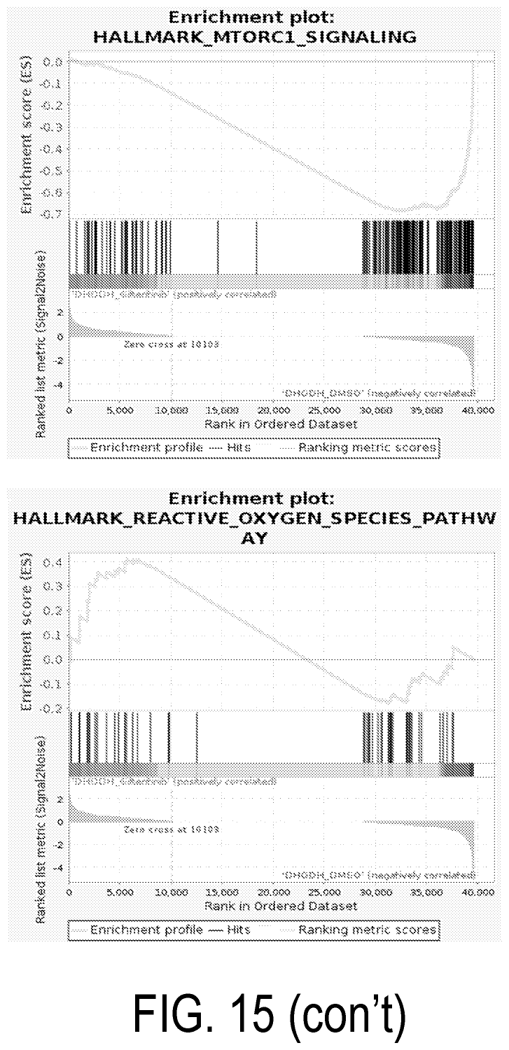

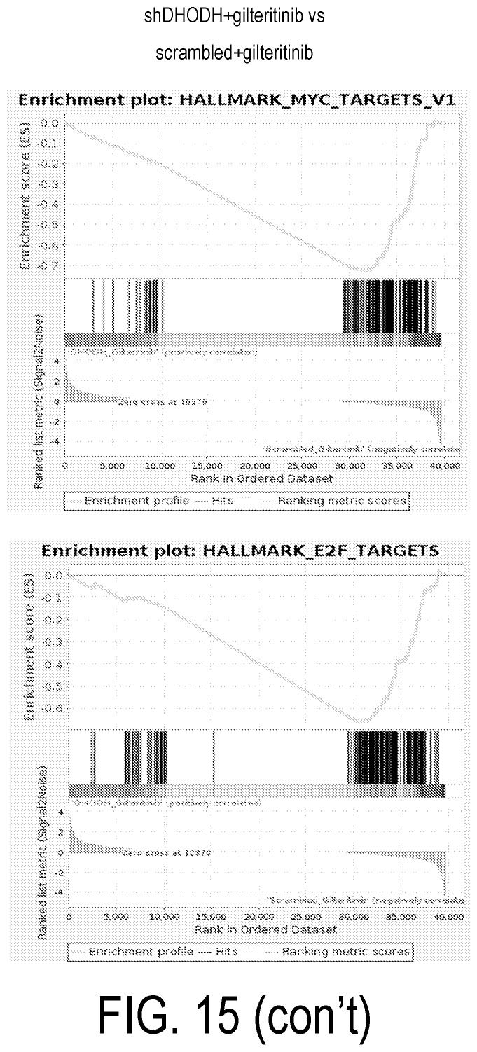

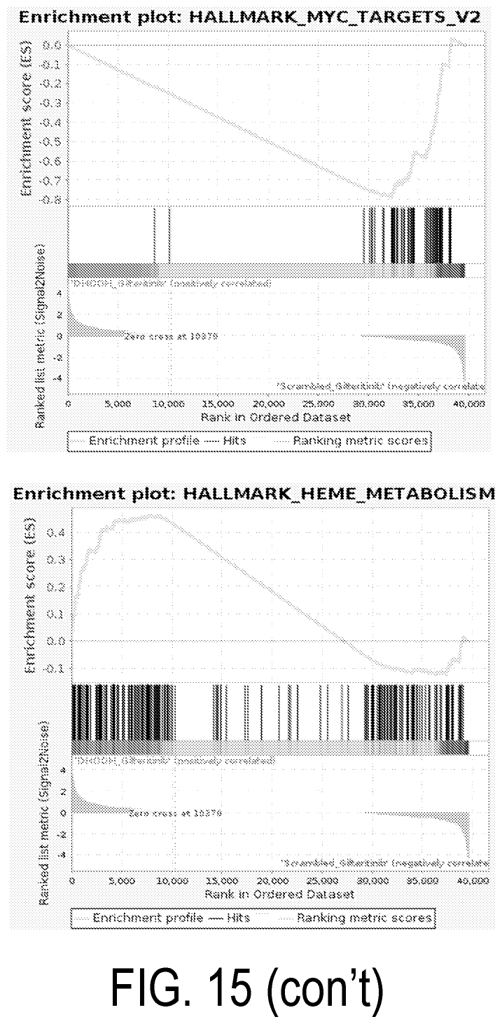

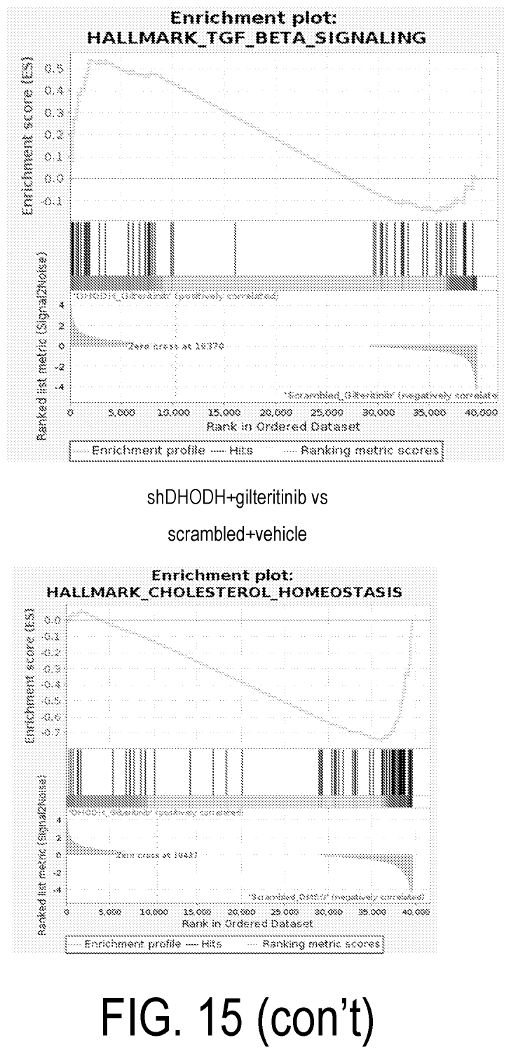

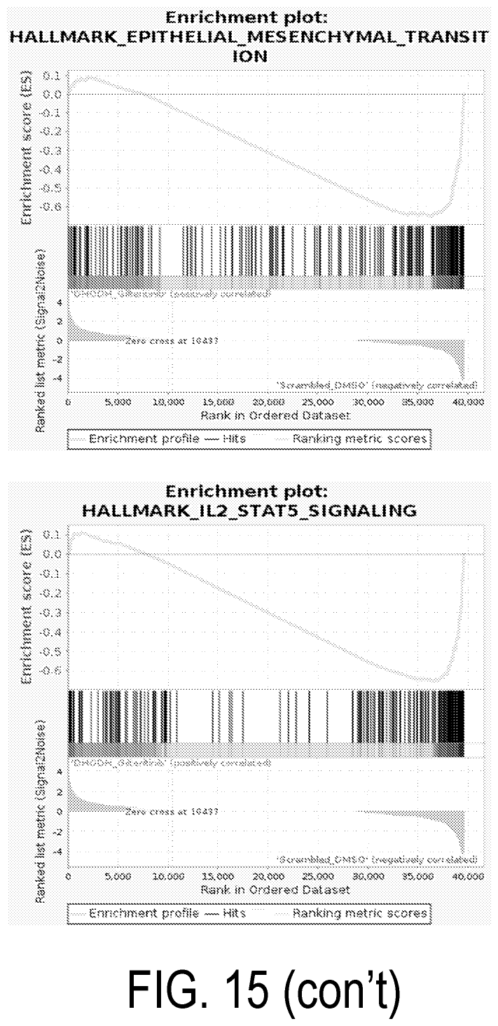

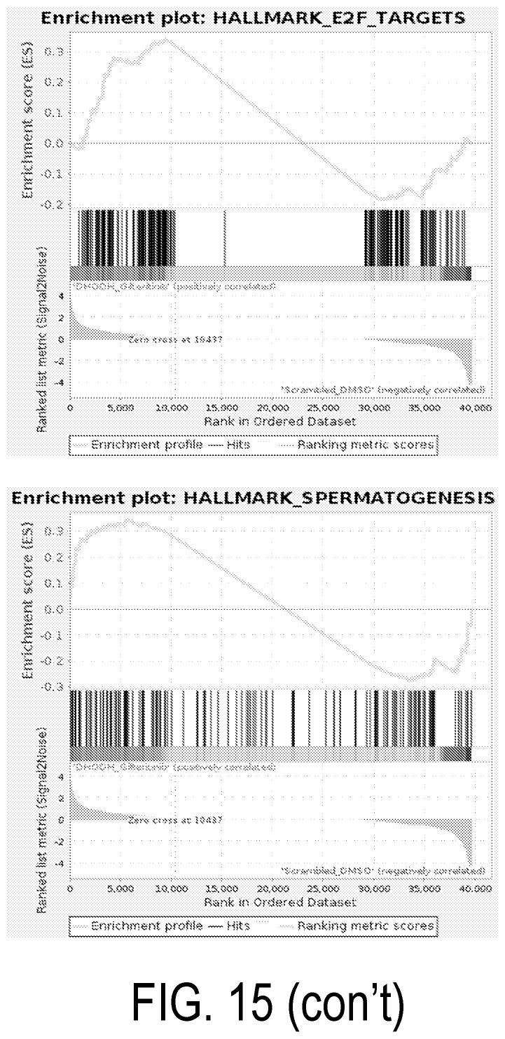

To facilitate comparative examination of pathway modulations by the different treatments, fully annotated Gene Set Enrichment Analysis (GSEA) hallmark gene sets for each comparison were analyzed by unsupervised hierarchical clustering based on their normalized enrichment scores (NES) (FIG. 11A-B). As a single agent, gilteritinib downregulated genes in glycolysis, Wnt signaling¹⁰ and Kras signaling, while concomitantly activating Myc pathway, OXPHOS, and fatty acid metabolism (FIG. 4A; FIGS. 11A-B, 12 and 13). Cholesterol homeostasis and the IL-2-STAT5 pathway, were also among the top pathways that were down-regulated whereas reactive oxygen species-related genes were found up-regulated by gilteritinib treatment alone (FIGS. 14 and 15). On the other hand, combination treatments substantially altered the landscapes of enriched gene sets in comparison with gilteritinib or gene-targeting shRNA alone, implying that combination profoundly disturbed the directionality of gene expression and pathway activities.

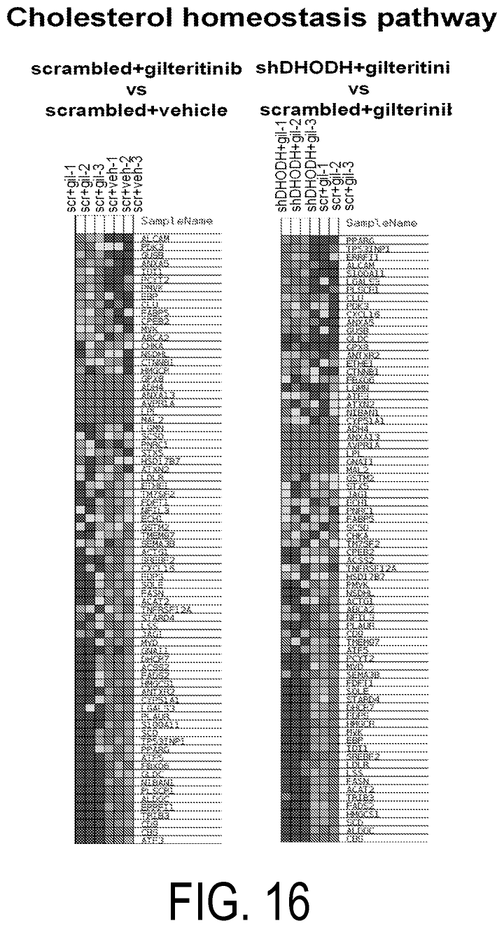

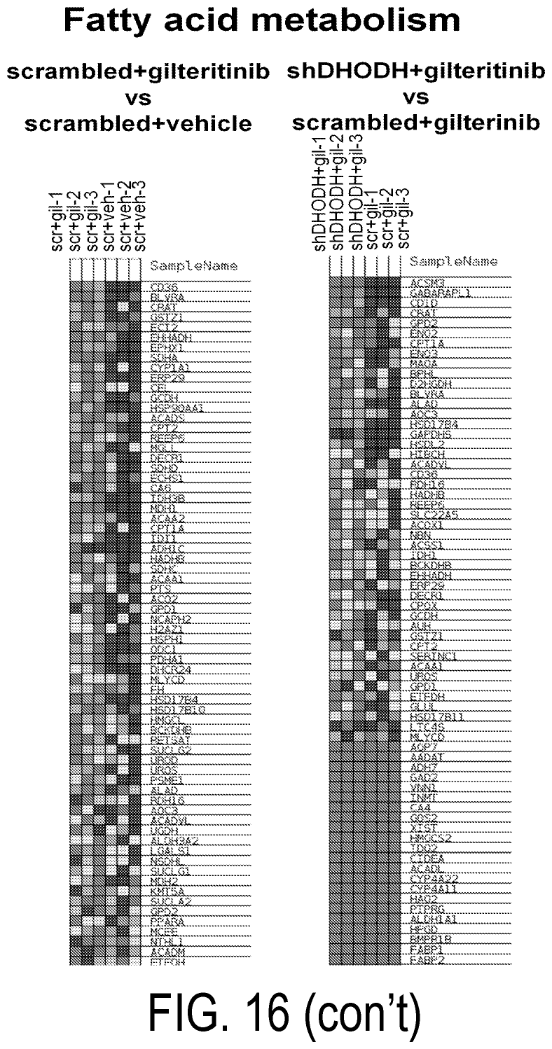

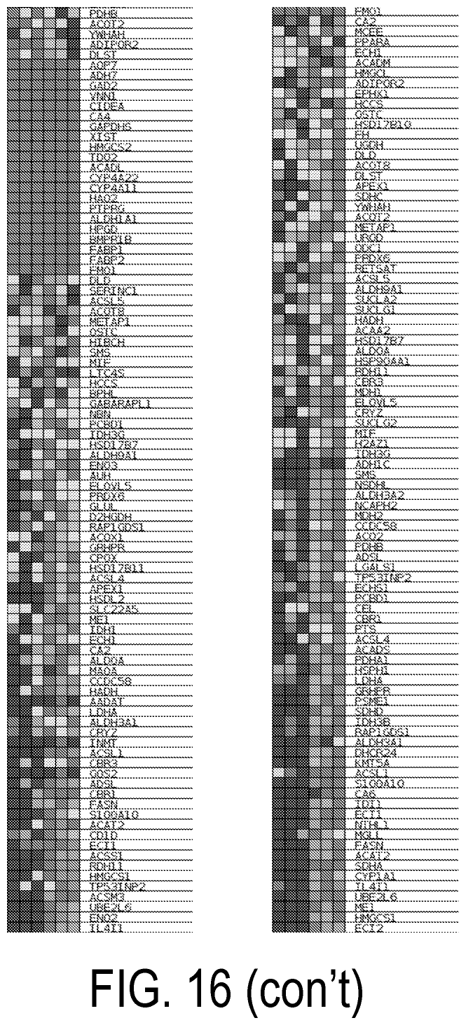

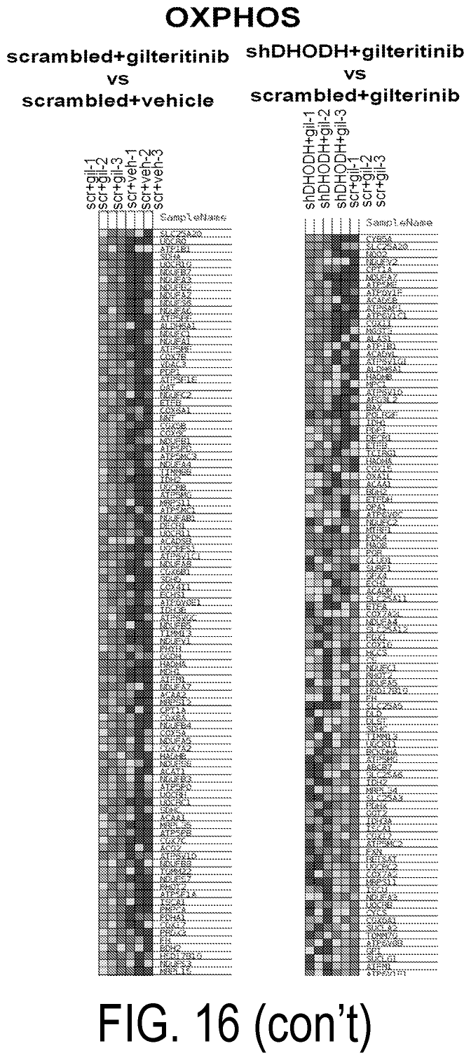

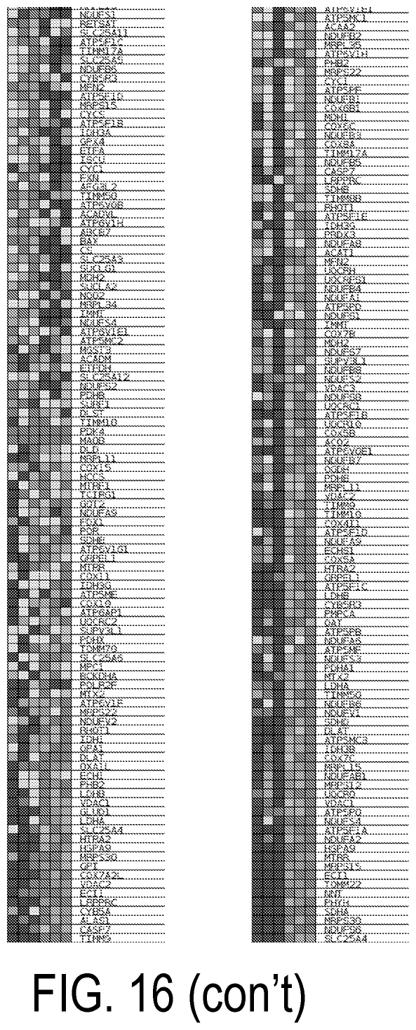

To gain insights into signaling network dynamics, GSEA C5 Oncology gene set and IPA analyses were conducted. In the shCDK9/gilteritinib group, both analyses robustly predicted cell division-related processes, such as: kinetochore metaphase pathway, mitotic roles of polo-like kinase, DNA replication, and chromosome segregation, as top affected pathways (FIG. 16A-B), echoing the observed large fold depletion of the abundance of gene transcripts in mitosis and kinetochore formation, including SKA1, KIFC1 and KIF15 (FIG. 3E). The DNA replication, kinetochore formation and mitochondrial electron transport chain pathways were also identified when gene sets involved in the synergistic effect of shCDK9 and gilteritinib were functionally categorized with Cytoscape enrichment maps (FIG. 4B; FIG. 16C). Heatmap analysis of transcripts in top GSEA gene sets demonstrated that the transcriptional profile of combination therapy is considerably different from those of monotherapies or vehicle control (FIG. 4C). These findings show that shCDK9/gilteritinib combination treatments predominantly blunt mitosis and cell cycle progression in addition to metabolic rewiring.

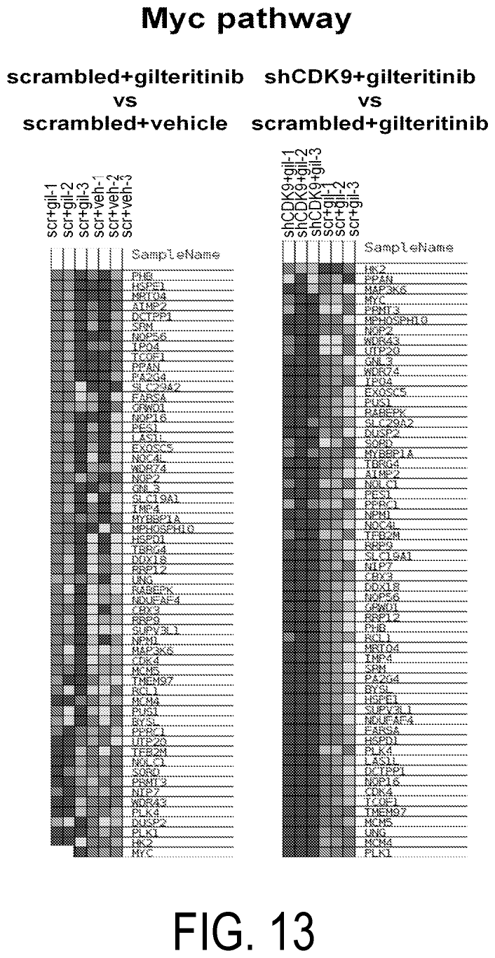

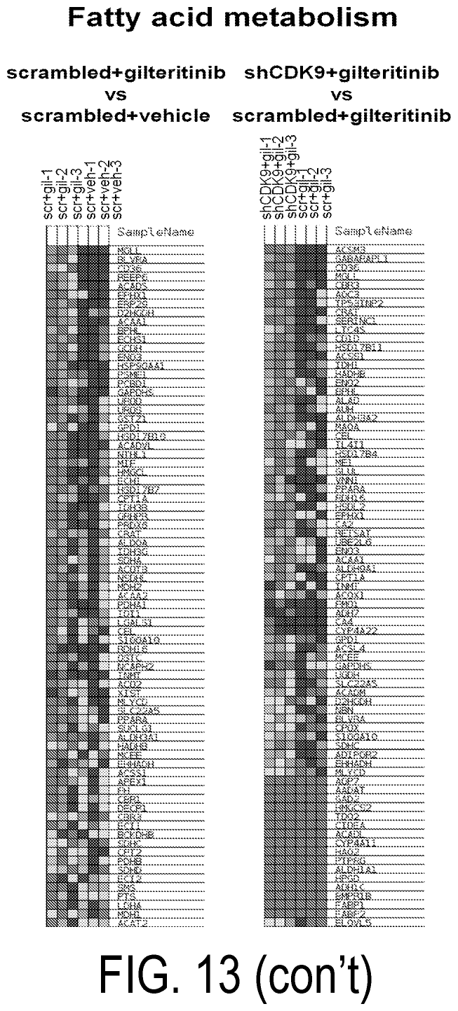

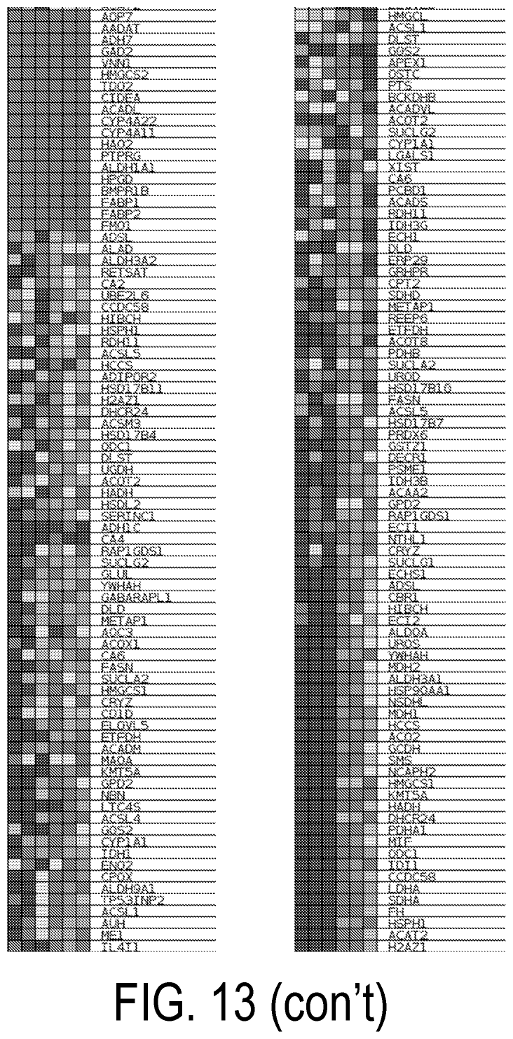

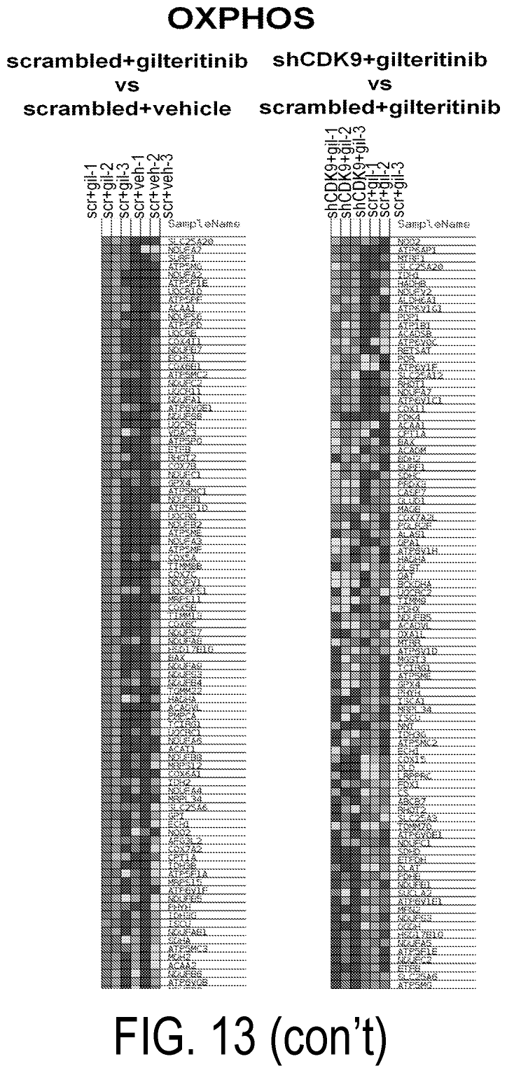

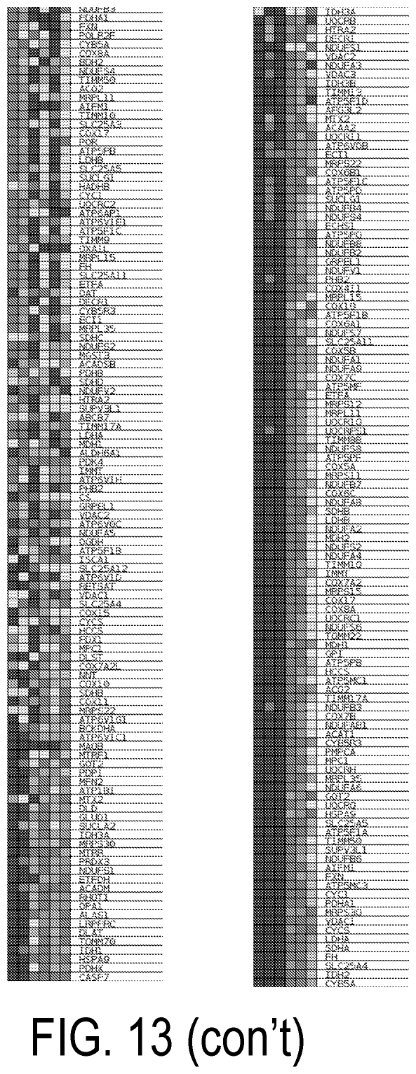

In shDHODH/gilteritinib group, it was found that the gilteritinib-induced OXPHOS, fatty acid metabolism and Myc pathways were among the top pathways downregulated by shDHODH/gilteritinib treatment (FIG. 4D; FIG. 11B; FIG. 13). Cholesterols and steroids are produced from the mevalonate pathway¹¹. The steroid metabolic process was found enriched by IPA analysis and Cytoscape analysis (FIG. 4E-F; FIG. 16D-F). Leading-edge analysis of the GSEA cholesterol homeostasis gene set highlighted genes that were strongly downregulated in combination-treated cells, including CBS, ALDOC, and HMGCSI (FIG. 13). This finding provides strong evidence that shDHODH and gilteritinib cooperatively weaken steroid biosynthesis by suppressing the mevalonate pathway.

Interestingly, there are some common features altered by both shDHODH/gilteritinib and shCDK9/gilteritinib combinations. For instance, the expression of key OXPHOS-related genes (FH which encodes fumarase and SDHA which encodes succinate dehydrogenase) and Myc pathway-related genes (PLK1, PLK4 and Myc) was upregulated by gilteritinib treatment, yet markedly reduced by both shCDK9/gilteritinib and shDHODH/gilteritinib combination treatments (FIGS. 4A and D; FIGS. 11A-B, 12 and 13).

Taken together, genetic deletion of CDK9 or DHODH sensitizes AML cells to gilteritinib treatment by converging on transcriptional suppression of Myc pathway, OXPHOS and related biosynthetic metabolism. A previous study suggested that depletion of HK2, a hexokinase isoform highly expressed in cancer, elevates OXPHOS, sensitizing tumor cells to cell death mediated by growth factor deprivation¹². In line with this, 28 genes in glycolysis (like HK2, HK3 and PFKFB3) were positively enriched in CRISPR screen of cell fitness to gilteritinib¹³ (FIG. 4G), implying that silencing these genes provides survival benefit for gilteritinib-treated cells which are adapted to OXPHOS and biosynthetic metabolism.

### The Synergistic Interactions of Gilteritinib and Ablation of Co-Essential Genes Converge on Inactivation of a Common OXPHOS Pathway to Deplete Metabolites in Purine Synthesis Mevalonate Metabolism

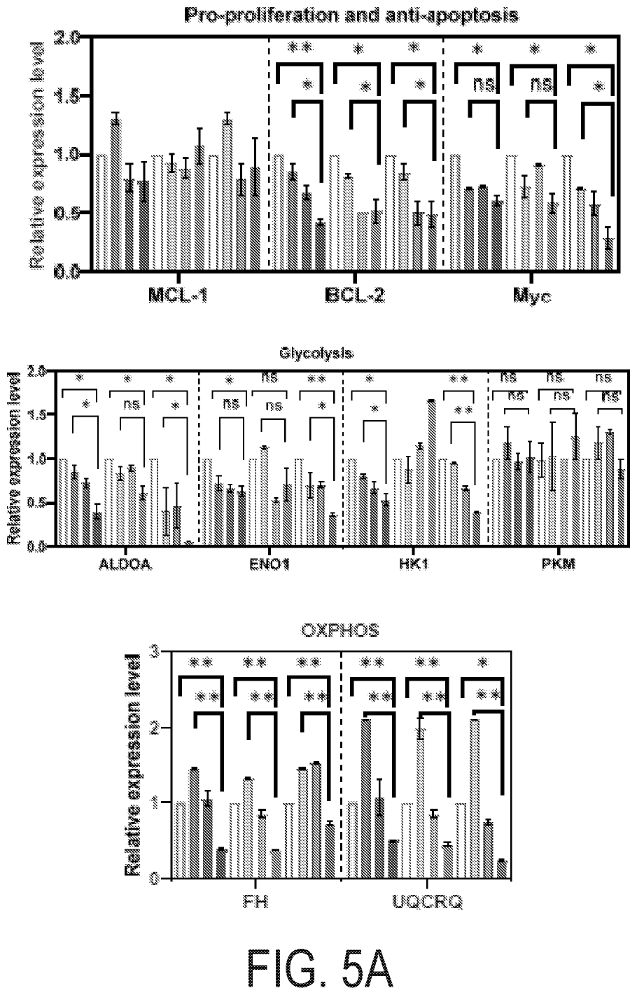

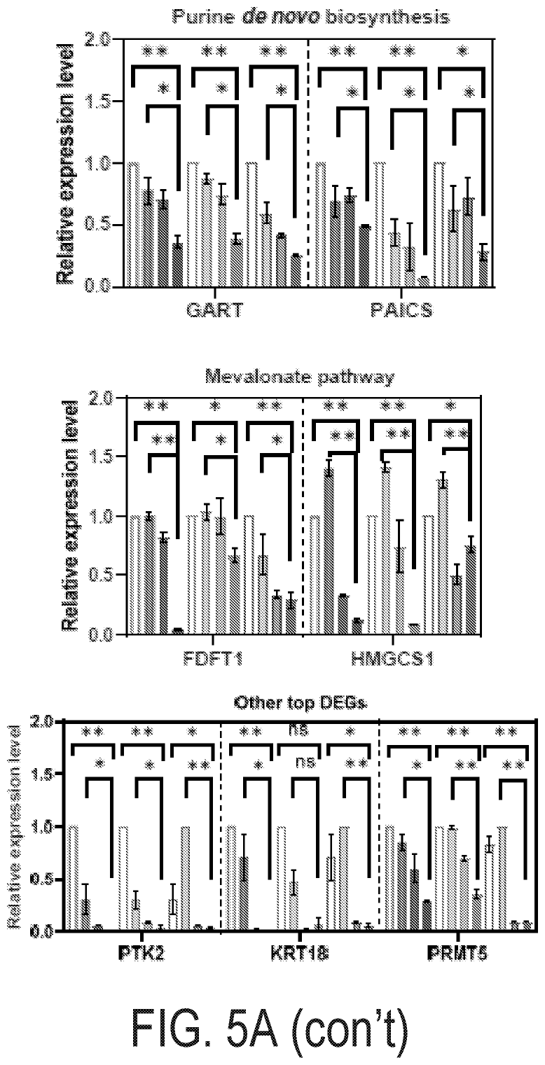

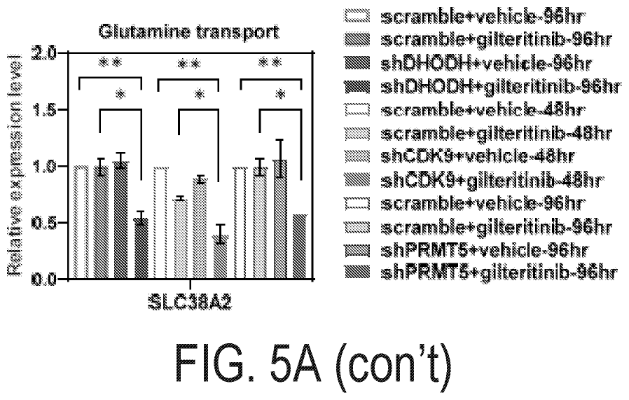

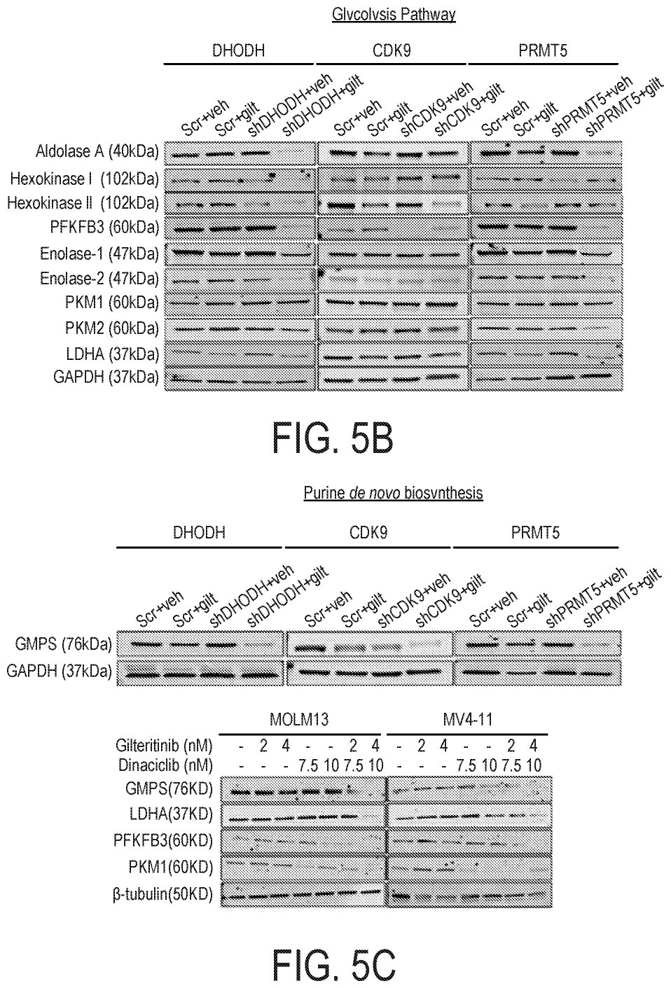

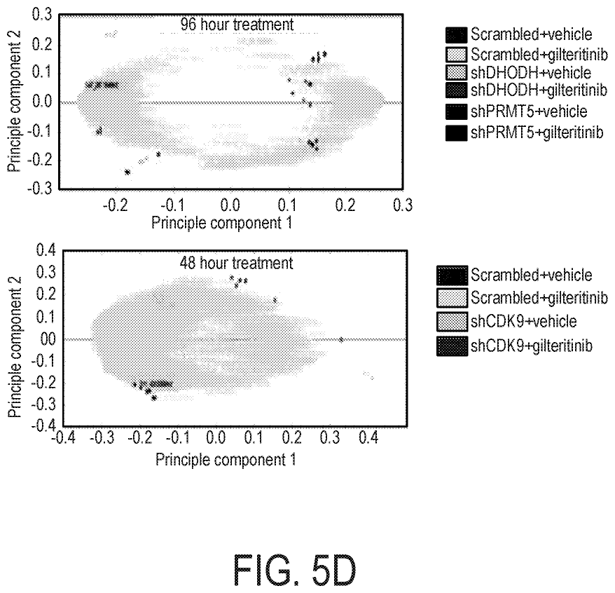

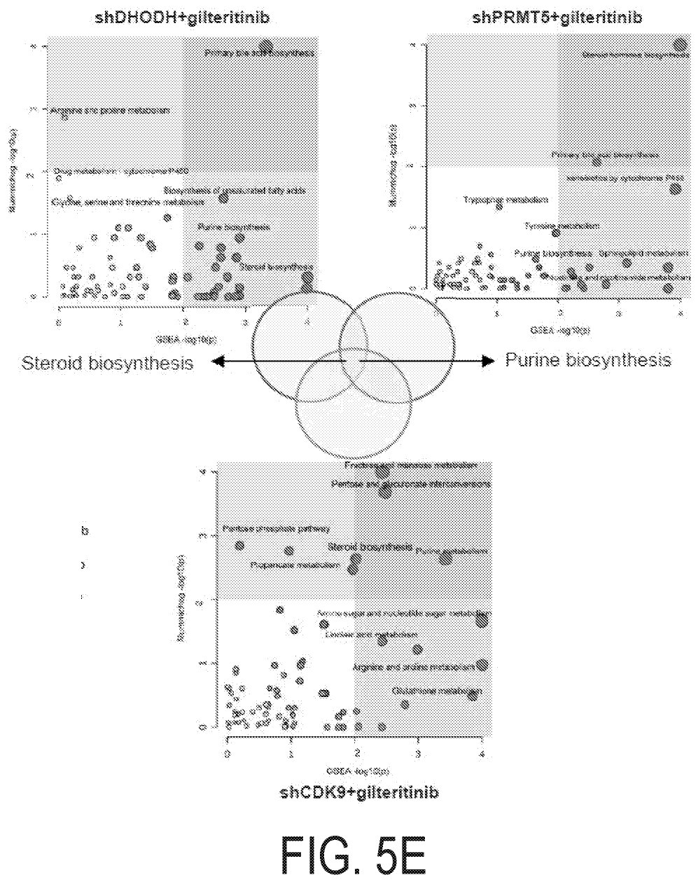

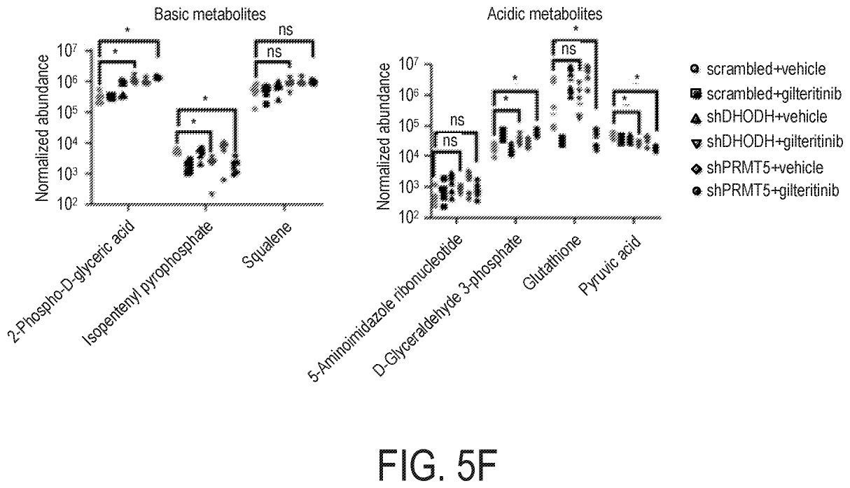

To evaluate the effect of combination treatments on mitochondrial activities, mitochondrial depolarization was measured by tetramethylrhodamine, methyl ester (TMRM). The frequencies of TMRM⁺ cells were drastically reduced in CDK9, DHODH or PRMT5-depleted cells compared with scrambled control in the presence of a sublethal dose of gilteritinib, showing that combination treatment leads to loss of mitochondrial depolarization (FIG. 10). To further understand the mechanism of synergy, the expression of genes involved in apoptotic and mitochondria-related metabolic pathways shared by the CRISPR screen, shCDK9/gilteritinib RNA-seq and shDHODH/gilteritinib RNA-seq data sets by real-time PCR (FIG. 3G) were measured. The expressions of BCL-2 and Myc were remarkably attenuated by shRNA-mediated knockdown of CDK9, DHODH, or PRMT5, suggesting that CDK9, DHODH and PRMT5 promote anti-apoptotic signaling (FIG. 5A). Conversely, MCL-1 expression was not affected by combination treatments. Purine de novo biosynthesis, mevalonate pathway and OXPHOS are the primary mitochondria-associated metabolic pathways¹⁴. Hallmarks of these pathways, phosphoribosylaminoimidazole carboxylase (PAICS), phosphoriboslglycinamide formyltransferase (GART), farnesyl-diphosphate farnesyltransferase 1 (FDFT1), hydroxymethylglutaryl-CoA synthase (HMGCS1), fumarase (FH) and ubiquinol-cytochrome C reductase complex III subunit VII (UQCRQ) consistently underwent depletion in diverse combined treatment conditions (FIG. 5A).

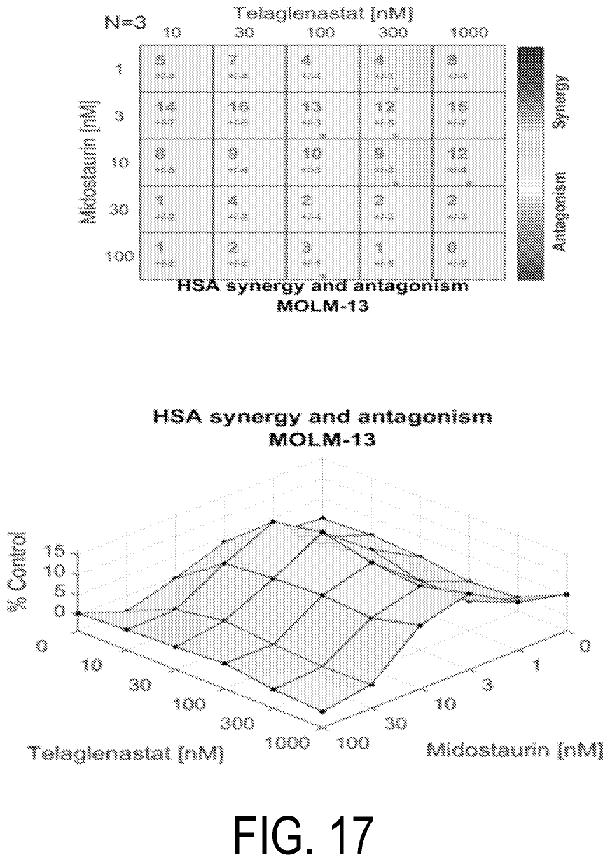

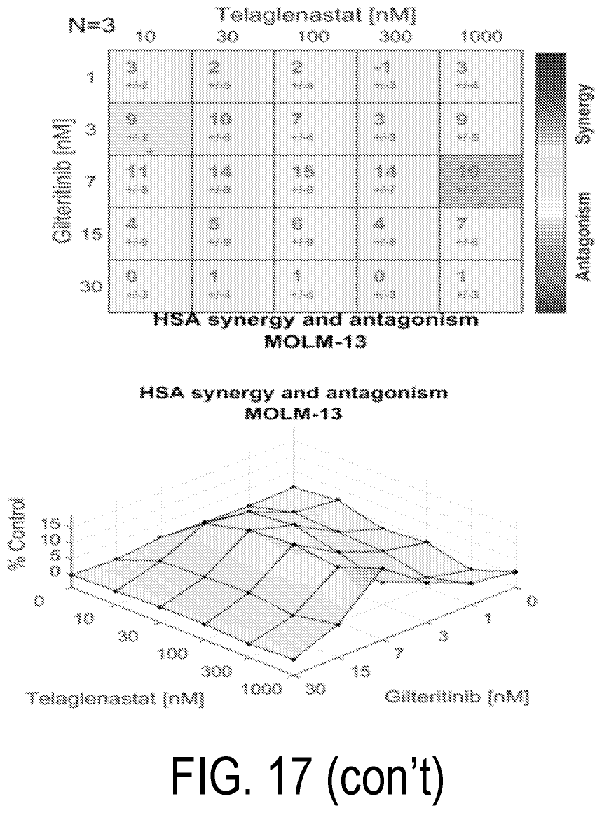

Of note, knockdown of CDK9, DHODH or PRMT5 significantly decreased SLC38A2 expression in gilteritinib-treated MOLM-13 cells, while having negligible effects in untreated cells (FIG. 5A). Previous reports showed that glutamine metabolism, via its capacity of supporting both mitochondria function and cell redox metabolism, is a metabolic dependency of FLT3-ITD AML¹⁵. In agreement with this, the glutaminase inhibitor, telaglenastat, shows synergistic cytotoxic effect with midostaurin or gilteritinib on AML cells (FIG. 17). FLT3-ITD inhibition reduces glutaminolysis by blocking glutamine influx through SLC1A5, the primary glutamine transporter^{15,16}. SLC38A2 can serve as a redundant glutamine transporter to compensate the deficiency of SLC1A5¹⁷. Therefore, these results show a mechanism of action where simultaneous inhibition of FLT3-ITD and co-essential targets (CDK9, PRMT5, and DHODH) can induce AML cell starvation due to the elimination of SLC1A5 and SLC38A2-mediated amino acid transport.

At the protein level, knockdown of DHODH, CDK9 or PRMT5 consistently decreased the expressions of aldolase A, GMPS, LDHA, and PFKFB3 in gilteritinib-treated cells compared with scrambled control (FIG. 5B). The expression of hexokinase II was predominantly downregulated by gilteritinib treatment alone. Knockdown of CDK9 did not significantly alter the expressions of enolase-1, enolase-2, PKM1/2 and hexokinase I in cells being exposed to vehicle or gilteritinib (FIG. 5B). Combined dinaciclib and gilteritinib treatment reduced GMPS, LDHA, PFKFB3 and PKM1 expression in a dose-dependent manner, phenocopying the genetic knockout effect (FIG. 5C).

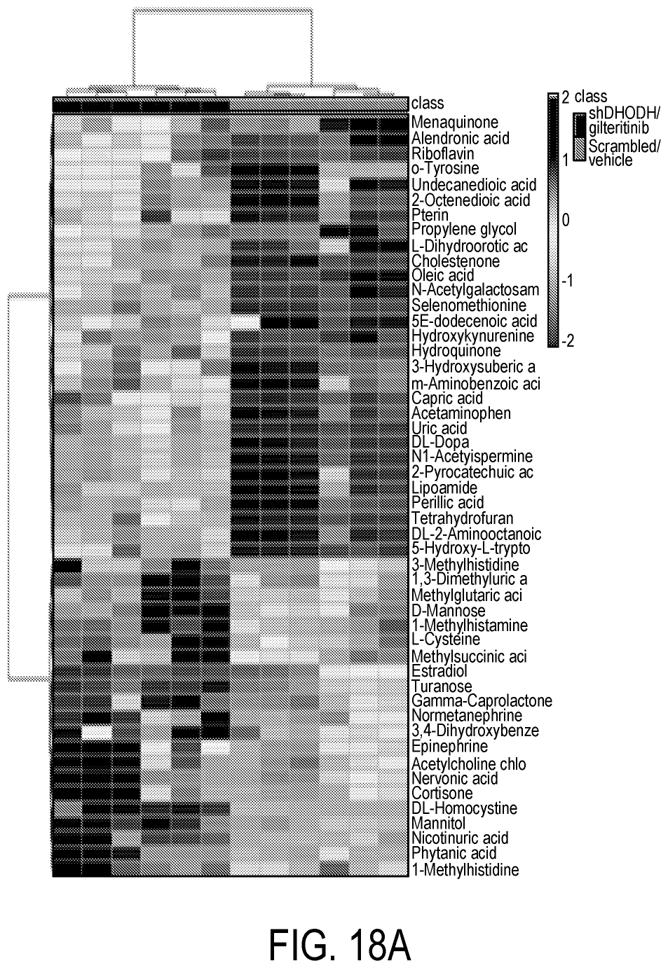

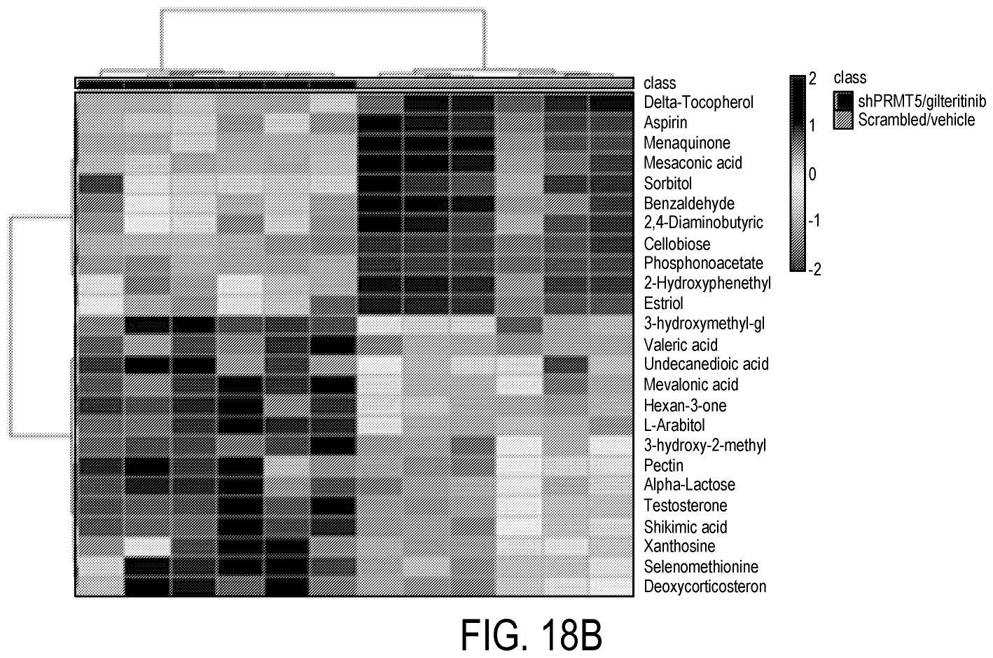

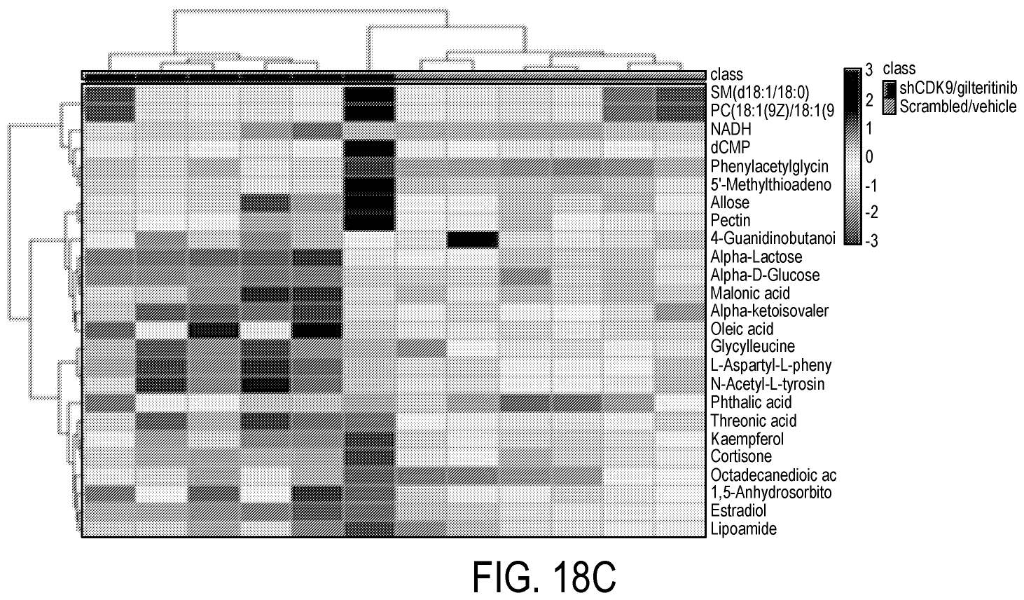

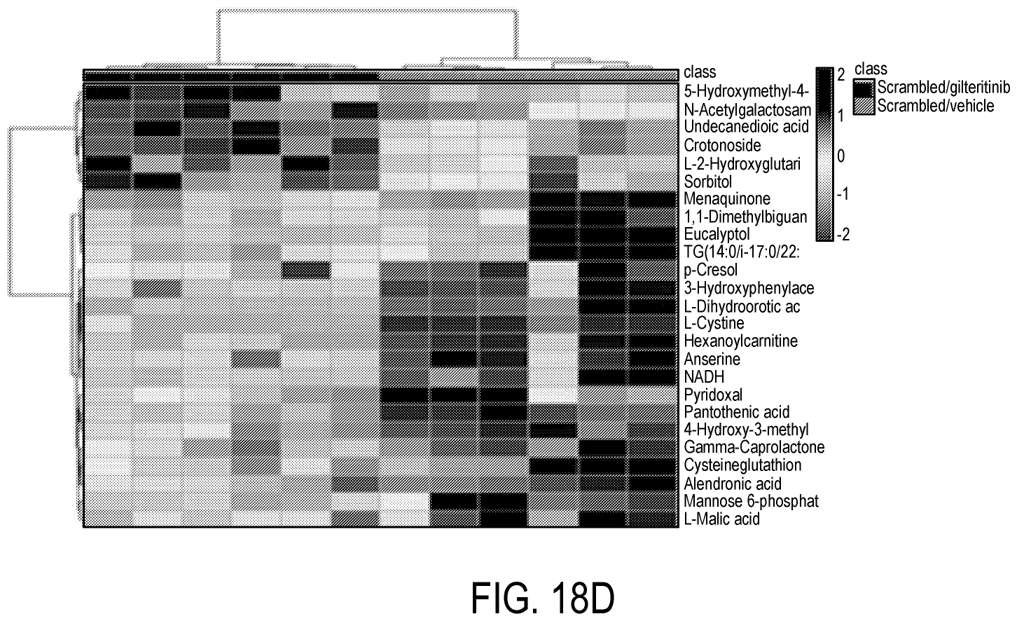

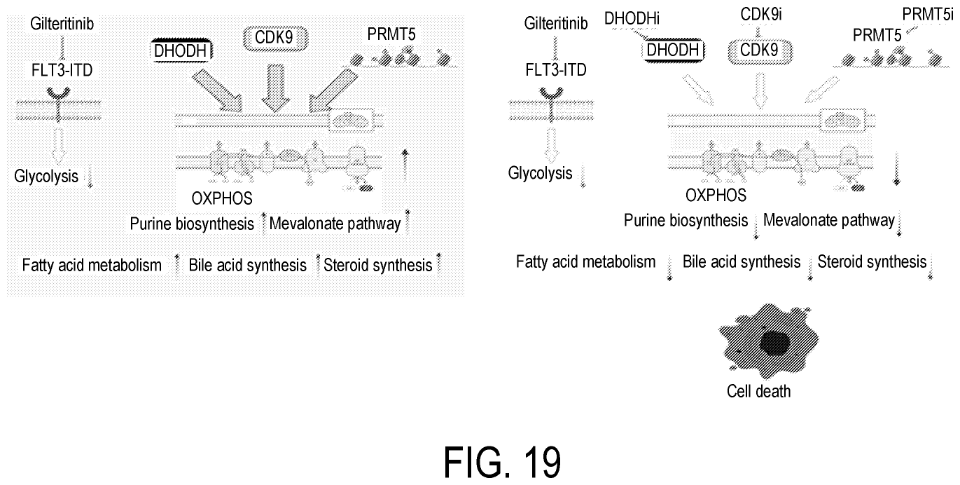

The metabolic rewiring associated with three combination treatments was further investigated by using metabolomics profiling. Principal components analysis of metabolites and heatmap analysis of the different treatment groups reveal that cells which received combined treatment exhibited distinct metabolic profiles (FIG. 5D; FIG. 18). GSEA and Mummichog pathway analysis revealed that gilteritinib treatment led to negative enrichment of pentose phosphate pathway, amino acid metabolism and glycolysis/glyconeogenesis and positive enrichment of steroid metabolism, purine biosynthesis, bile acid metabolism and fatty acid biosynthesis (FIG. 5E). Conversely, steroid biosynthesis and purine biosynthesis are among the top pathways which were downregulated by combined treatments. In response to combined treatments, metabolic intermediates in glycolysis, 2-phospho-D-glyceric acid and D-glyceraldehyde 3-phosphate, were accumulated and final product pyruvic acid was concurrently depleted (FIG. 5F), echoing the downregulation of key enzyme expression (aldolase and hexokinase I) as revealed by RNA-seq. Combination treatments also resulted in remarkable reduction in the abundance of mevalonate pathway metabolites, isopentenyl pyrophosphate, which may arrest downstream cholesterol and steroid synthesis reaction. This provides further evidence for combined treatment-mediated OXPHOS deficiency and related energy starvation.

Collectively, gilteritinib treatment shifts metabolic dependency from aerobic glycolysis to OXPHOS, thus rendering OXPHOS dependent cells sensitive to inhibition of CDK9, DHODH, or PRMT5.

Pharmacologic Validation Confirms Synergy of Several Targets with Gilteritinib

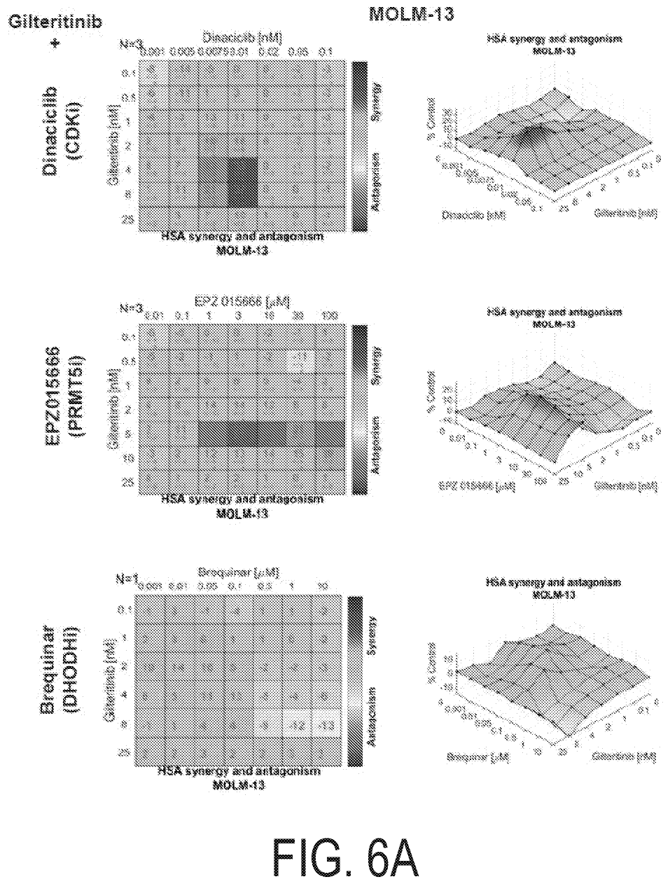

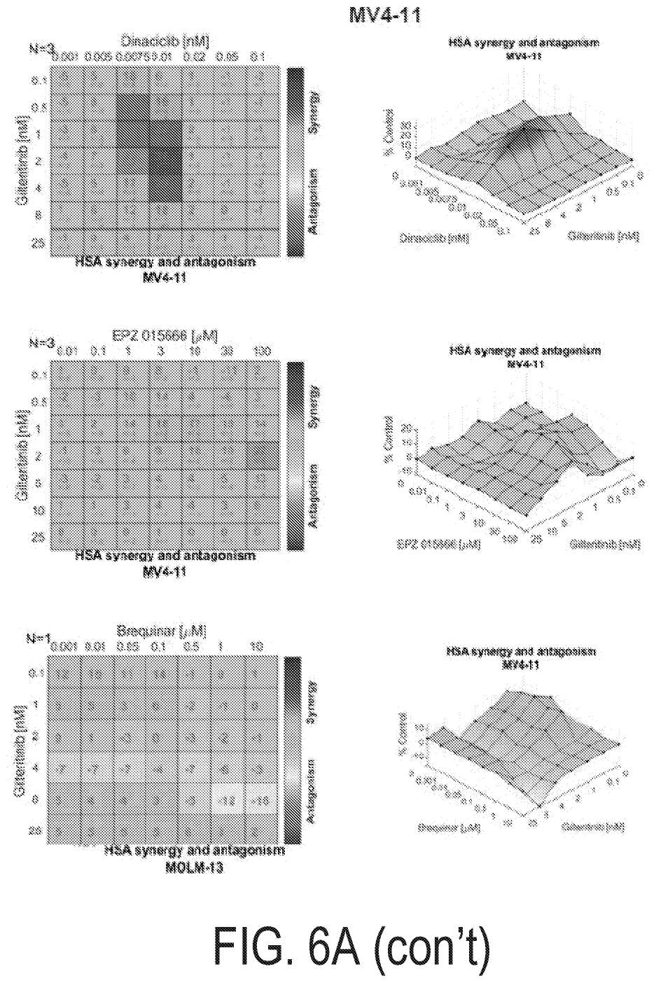

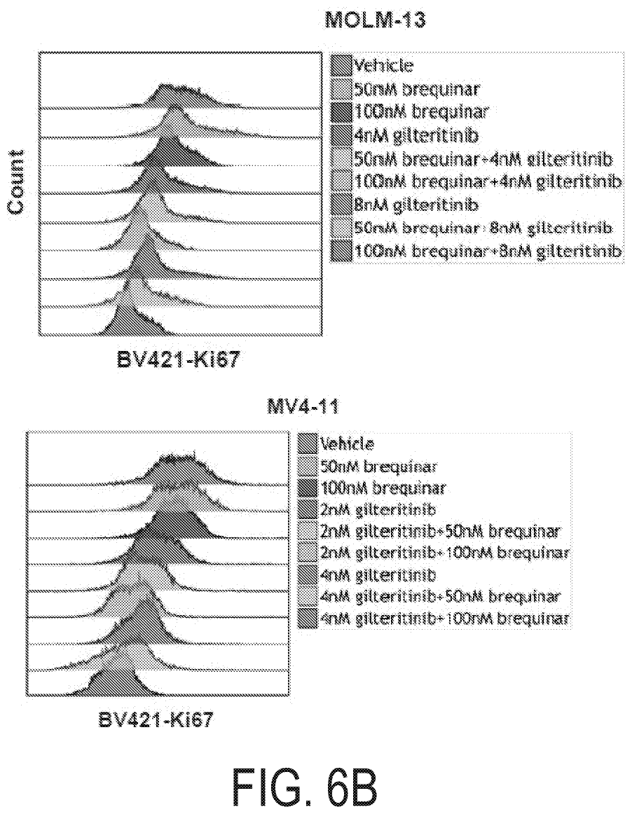

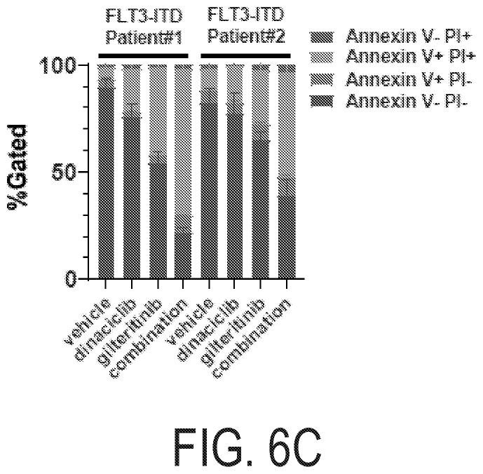

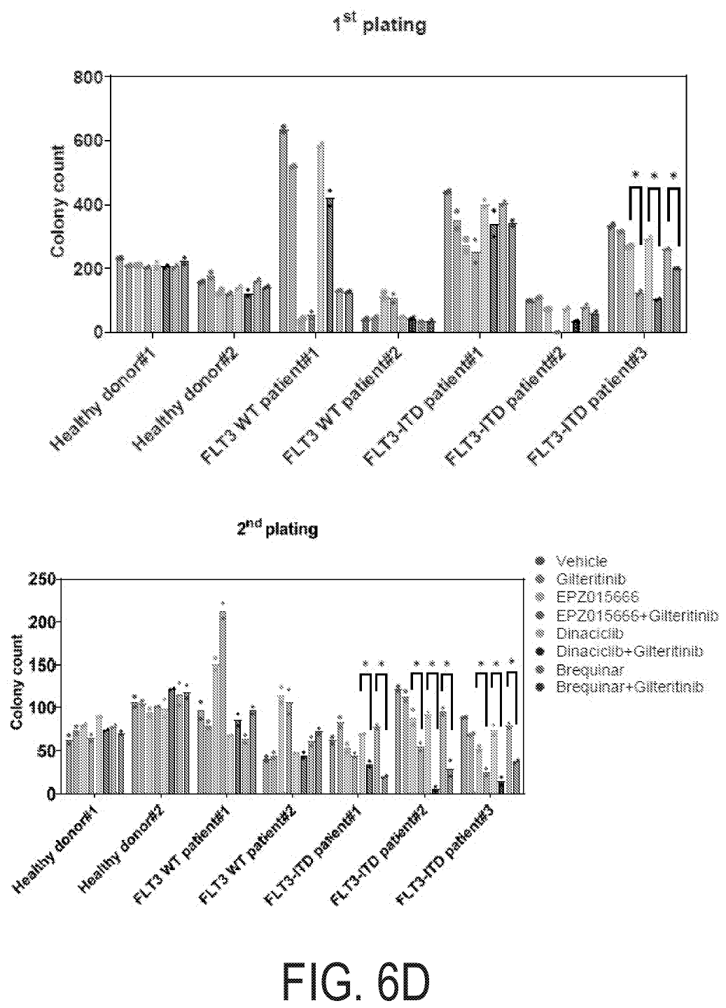

Next, it was determined whether pharmacologic inhibition of CDK9, DHODH or PRMT5, using commercially available inhibitors (brequinar for DHODH, dinaciclib for CDK1/2/5/9, and EPZ015666 for PRMT5) is synergistic with gilteritinib in AML cells. For MOLM-13 cells, synergy ranges were determined to be 2-10 nM gilteritinib with 0.0075-0.01 nM dinaciclib or 1-100 μM EPZ015666 (FIG. 6A). For MV4-11 cells, maximum synergy was observed at 0.1-8 nM gilteritinib in combination with 0.0075-0.01 nM dinaciclib or 3-100 μM EPZ015666 (FIG. 6A). Synergy ranges for each drug are physiologically achievable and represent promising opportunities for future combination treatments^{18,19} Brequinar and gilteritinib combination showed modest synergy with regards to MOLM-13 and MV4-11 viability (FIG. 6A). Instead, brequinar and gilteritinib synergistically suppressed cell proliferation as indicated by decreased Ki67 staining (FIG. 6B). Results in patients FLT3-ITD bearing cells were similar: gilteritinib and dinaciclib synergistically induced cell death by overcoming stroma protection (FIG. 6C). To determine the effect of diverse inhibitor combinations on self-renewal of primary cells, cells from three FLT3-ITD patients, two FL73-WT patients and two healthy donors were seeded into semi-solid media in the presence of vehicle, 8 nM gilteritinib, 100 μM EPZ015666, 0.1 nM dinaciclib, 100 nM brequinar, combination of gilteritinib and EPZ015666, combination of gilteritinib and dinaciclib or combination of gilteritinib and brequinar. At the first plating, only FLT3-ITD patient #3 grew significantly fewer colonies in response to combination therapies (FIG. 6D). However, at the secondary plating, combination treatments robustly abolished colony formation of cells of all FLT3-ITD patients but not cells of FLT3-WT patients. Therefore, pharmacologic inhibition of CDK9, DHODH or PRMT5 could recapitulate the effects of the genetic knockdown to confer sensitivity to gilteritinib treatment.

### Combination Treatment of Dinaciclib and Gilteritinib Prolongs Survival in an Aggressive Xenograft Mouse Model

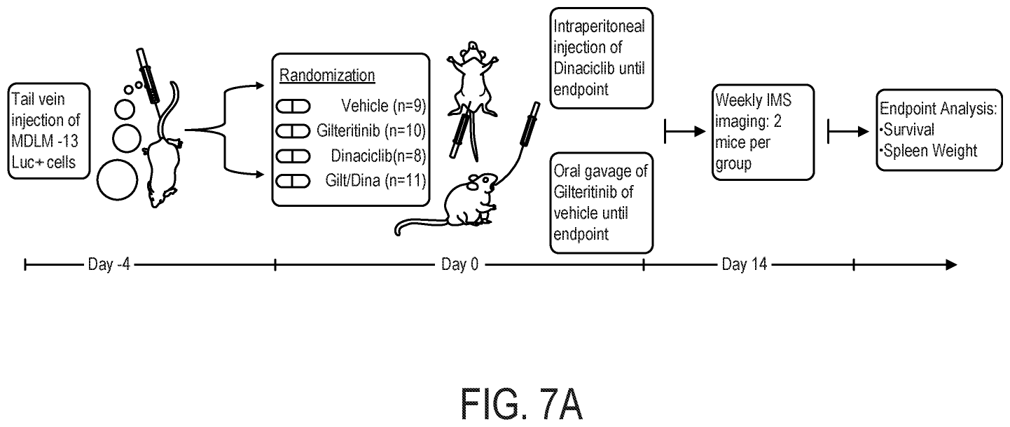

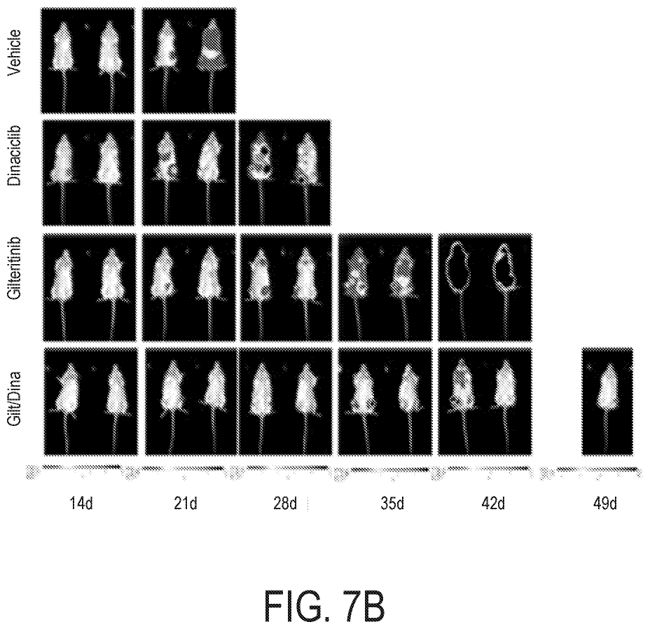

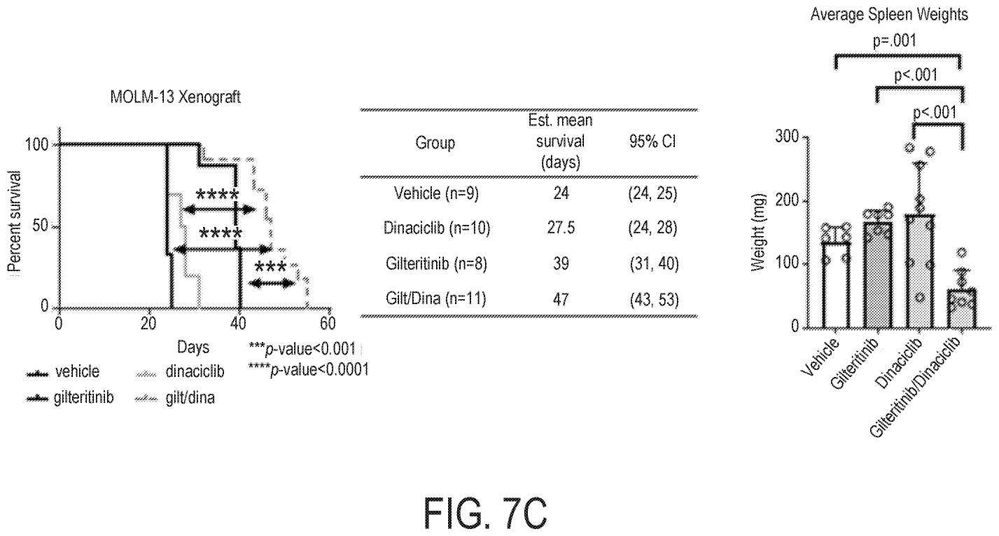

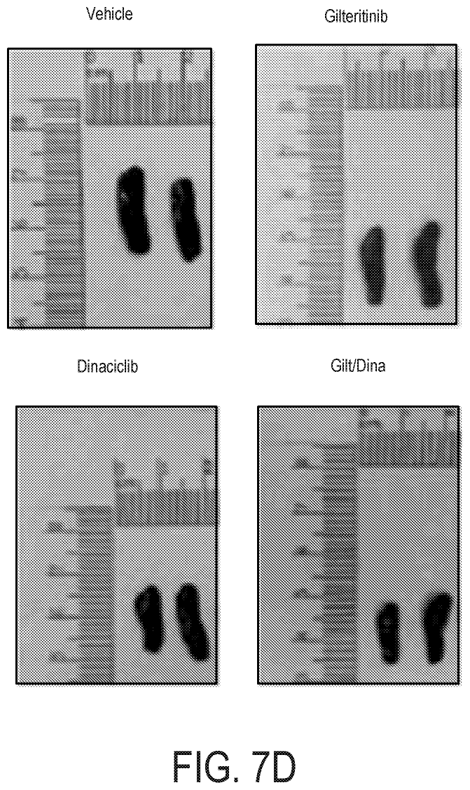

To assess the translational relevance of the gilteritinib/dinaciclib synergy, a MOLM-13-Luc⁺ engraftment mouse model was employed (FIG. 7A). At each given time point, AML disease burden in the combination group was significantly lower than that of the monotherapy or vehicle group (FIG. 7B). As single agents, dinaciclib or gilteritinib, provided marginal survival advantage to leukemic mice compared with vehicle control, while the combination arm displayed a significant survival benefit relative to vehicle or both single agent arms (mean survival time: 47 days for combination, 39 days for gilteritinib, 27.5 days for dinaciclib and 24 days for vehicle; combination vs gilteritinib: p-value<0.001) (FIG. 7C). At endpoint, a substantial reduction in spleen weight in the combination therapy group over all other treatment arms was observed (FIG. 7D). This experiment provides strong evidence that the combination therapy of gilteritinib and dinaciclib at clinically relevant doses shows appealing in vivo efficacy.

### Discussion

In this study, PRMT5, CDK9, and DHODH were identified as novel synergistic lethal partners with gilteritinib in FLT3-ITD AML. It was shown that genetic deletion and pharmacological inhibition of these targets sensitize AML cell lines and primary patient samples to gilteritinib treatment and that the cyclin-dependent kinase inhibitor, dinaciclib, in combination with gilteritinib, reduces disease burden and provides survival benefits in an AML xenograft mouse model.

PRMT5, CDK9, and DHODH play different roles in activating proliferation and inhibiting apoptosis. DHODH is the rate limiting enzyme of the de novo pyrimidine synthesis pathway, converting dihydroorotate (DHO) to orotate^{20,21}. Inhibition of DHODH induces differentiation of diverse AML subtypes²². PRMT5 catalyzes symmetric demethylation of histone arginine to induce gene silencing²³. PRMT5 also methylates and regulates proteins involved in diverse cellular processes, including transcription, translation, and apoptosis. PRMT5 inhibition has been shown to kill AML cells²⁴⁻²⁶. CDK9 inhibitors downregulate MCL-1 to induce cell death in AML, overcoming MCL-1-dependent drug resistance^{27,28}. In addition, CDK9 inhibition suppresses the expression of relevant MYB target genes including BCL2 and CCNB1²⁹. CDK9 inhibitors were also shown to inhibit active phospho-TEFb and the expression of E2F target genes necessary for the G1/S transition, DNA replication and mitotic activity. Myc, a critical downstream transcriptional target of phospho-TEFb, was shown to be responsible for CDK9-mediated cell proliferation and survival. In agreement with these observations, our data show that kinetochore mitotic spindle, chromosome remodeling, Myc pathway and G2M cell cycle checkpoint gene sets are all significantly downregulated with CDK9 knockout and that these shCDK9-induced effects are further strengthened by FLT3 inhibition.

It was found that gilteritinib-treated AML cells are addicted to OXPHOS rather than aerobic glycolysis for energy production and biosynthesis reactions, which renders these metabolically adapted cells extremely vulnerable to transcriptional silencing of the components of mitochondrial electron transport chain complexes. This is in line with previous reports suggesting that therapy-resistant AML cells increase their mitochondrial mass and have high OXPHOS³⁰. Cells harboring FLT3-ITD demonstrated a highly glycolytic phenotype and had central carbon metabolism elevated by regulating FOXO activity³¹. The gene set enrichment analysis reveals that glycolysis is predominantly downregulated by gilteritinib monotherapy. This is supported by previous studies showing that glycolytic enzymes were pronouncedly suppressed by the FLT3 inhibitor, AC220¹⁵. More importantly, this study shows that knock out of each of 28 glycolytic genes (including HK2) confers resistance to gilteritinib treatment. Upon HK2 depletion, glucose flux to pyruvate and lactate is suppressed, but TCA fluxes and OXPHOS are maintained. Coupling glycolysis deficiency with elevated OXPHOS promotes leukemia growth. The mechanism of CDK9, DHODH and PRMT5 controlling OXPHOS remains unknown and warrants further exploration. However, our data suggests that it is Myc dependent, since the Myc pathway is activated by gilteritinib treatment which can act by transcriptionally upregulate the genes in OXPHOS to provide drug resistance^{32,33}.

Dysregulation of the mevalonate pathway has been implicated in multiple aspects of tumor progression³⁴. The end product of this pathway, cholesterol, is an important component of cellular membranes and serves as a precursor for steroid hormones and vitamin D. The rate-limiting step of the mevalonate pathway is controlled by HMGCSI, an enzyme that converts HMG-COA to mevalonate and is the target of cholesterol-reducing statins. Our findings that that combinations of CDK9, PRMT5, or DHODH inhibition with gilteritinib inactivate the OXPHOS, purine biosynthesis and mevalonate pathway-mediated cholesterol/steroid synthesis suggests that all three forms of combination therapies converge on inhibition of these metabolic pathways to starve the leukemic cells. This is consistent with literature showing that blocking the purine de novo synthesis inhibits AML growth³⁵. In addition, administration of purine nucleobase derivatives rescues apoptotic effect of FLT3 inhibitors on MV4-11 cells³⁶. It was also reported that combination of the OXPHOS inhibitor IACS-010759 with the FLT3 inhibitor AC220 synergistically reduces glucose and glutamine enrichment, leading to impaired energy production and nucleotide synthesis³⁷.

Glutaminolysis, which is primarily mediated by glutaminase GLS and transporters (SLC1A5 and SLC38A2) plays important roles in AML by replenishing the TCA cycle intermediates. AML cells shunt carbon from glutaminolysis into citrate, feeding de novo fatty acid biosynthesis in the mitochondria and providing lipids for proliferating AML cells³⁸. Glutamine metabolism was shown to provide resistance to FLT3 inhibitor therapies 15 and the use of a GLS inhibitor in combination with either a FLT3 inhibitor or a BCL2 inhibitor effectively eliminates AML cells^{16,38}. These findings show that SLC38A2 expression can be downregulated by gilteritinib in combination with inhibitors for CDK9, PRMT5 and DHODH, highlighting the metabolic plasticity of AML. Interestingly, SLC38A2 ranked #6 out of 19115 total hits in a CRISPR screen with venetoclax in AML³⁹ showing that its inactivation is synthetic lethal with BLC-2 inhibition. Therefore, it is likely that glutamine transport is so critical for AML survival that any combinatorial treatments aimed at concurrently blocking glutaminolysis and BCL2 downregulation may enhance gilteritinib sensitivity.

In summary, this study shows gilteritinib-treated AML cells to be addicted to OXPHOS rather than aerobic glycolysis for energy production and biosynthesis reactions. CDK9-mediated mitotic spindle function and DHODH-mediated mitochondria metabolism are identified as additional transcriptional and metabolic dependencies in FLT3-ITD cells that are unmasked by FLT3 inhibitors. Loss of CDK9, PRMT5 and DHODH were demonstrated to induce metabolic adaptation and potentiate gilteritinib sensitivity in AML thus providing a rational for combining CDK9, PRMT5, DHODH or OXPHOS inhibitors to improve efficacy of FLT3 inhibitors.

### REFERENCES

1. Quentmeier H, Reinhardt J, Zaborski M, Drexler H G. FLT3 mutations in acute myeloid leukemia cell lines. Leukemia. 2003; 17 (1): 120-124.
2. Daver N, Schlenk R F, Russell N H, Levis M J. Targeting FLT3 mutations in AML: review of current knowledge and evidence. Leukemia. 2019; 33 (2): 299-312.
3. Ju H Q, Zhan G, Huang A, et al. ITD mutation in FLT3 tyrosine kinase promotes Warburg effect and renders therapeutic sensitivity to glycolytic inhibition. Leukemia. 2017; 31 (10): 2143-2150.
4. Perl A E, Martinelli G, Cortes J E, et al. Gilteritinib or Chemotherapy for Relapsed or Refractory FLT3-Mutated AML. N Engl J Med. 2019; 381 (18): 1728-1740.
5. Brinton L T, Sher S, Williams K, et al. Cotargeting of XPO1 Enhances the Antileukemic Activity of Midostaurin and Gilteritinib in Acute Myeloid Leukemia. Cancers (Basel). 2020; 12 (6).
6 Brinton L T, Zhang P, Williams K, et al. Synergistic effect of BCL2 and FLT3 co-inhibition in acute myeloid leukemia. J Hematol Oncol. 2020; 13 (1): 139.
7. Zhang P, Brinton L T, Williams K, et al. Targeting DNA Damage Repair Functions of Two Histone Deacetylases, HDAC8 and SIRT6, Sensitizes Acute Myeloid Leukemia to NAMPT Inhibition. Clin Cancer Res. 2021; 27 (8): 2352-2366.
8. Kolde R, Laur S, Adler P, Vilo J. Robust rank aggregation for gene list integration and meta-analysis. Bioinformatics. 2012; 28 (4): 573-580.
9. Wang B, Wang M, Zhang W, et al. Integrative analysis of pooled CRISPR genetic screens using MAGeCKFlute. Nature Protocols. 2019; 14 (3): 756-780.
10. Hou P, Wu C, Wang Y, et al. A Genome-Wide CRISPR Screen Identifies Genes Critical for Resistance to FLT3 Inhibitor AC220. Cancer Res. 2017; 77 (16): 4402-4413.
11. Li H Y, Appelbaum F R, Willman C L, Zager R A, Banker D E. Cholesterol-modulating agents kill acute myeloid leukemia cells and sensitize them to therapeutics by blocking adaptive cholesterol responses. Blood. 2003; 101 (9): 3628-3634.
12. DeWaal D, Nogueira V, Terry A R, et al. Hexokinase-2 depletion inhibits glycolysis and induces oxidative phosphorylation in hepatocellular carcinoma and sensitizes to metformin. Nat Commun. 2018; 9 (1): 446.
13. Smith J R, Hayman G T, Wang S J, et al. The Year of the Rat: The Rat Genome Database at 20: a multi-species knowledgebase and analysis platform. Nucleic Acids Res. 2020; 48 (D1): D731-D742.
14. Panina S B, Pei J, Kirienko N V. Mitochondrial metabolism as a target for acute myeloid leukemia treatment. Cancer Metab. 2021; 9 (1): 17.
15. Gallipoli P, Giotopoulos G, Tzelepis K, et al. Glutaminolysis is a metabolic dependency in FLT3 (ITD) acute myeloid leukemia unmasked by FLT3 tyrosine kinase inhibition. Blood. 2018; 131 (15): 1639-1653.
16. Gregory M A, Nemkov T, Reisz J A, et al. Glutaminase inhibition improves FLT3 inhibitor therapy for acute myeloid leukemia. Exp Hematol. 2018; 58:52-58.
17. Broer A, Rahimi F, Broer S. Deletion of Amino Acid Transporter ASCT2 (SLC1A5) Reveals an Essential Role for Transporters SNAT1 (SLC38A1) and SNAT2 (SLC38A2) to Sustain Glutaminolysis in Cancer Cells. J Biol Chem. 2016; 291 (25): 13194-13205.
18. Her N-G, Oh J-W, Oh Y J, et al. Potent effect of the MDM2 inhibitor AMG232 on suppression of glioblastoma stem cells. Cell Death & Disease. 2018; 9 (8): 792.
19. Gullà A, Hideshima T, Bianchi G, et al. Protein arginine methyltransferase 5 has prognostic relevance and is a druggable target in multiple myeloma. Leukemia. 2018; 32 (4): 996-1002.
20. Bajzikova M, Kovarova J, Coelho A R, et al. Reactivation of Dihydroorotate Dehydrogenase-Driven Pyrimidine Biosynthesis Restores Tumor Growth of Respiration-Deficient Cancer Cells. Cell Metab. 2019; 29 (2): 399-416.e310.
21. Boukalova S, Hubackova S, Milosevic M, Ezrova Z, Neuzil J, Rohlena J. Dihydroorotate dehydrogenase in oxidative phosphorylation and cancer. Biochim Biophys Acta Mol Basis Dis. 2020; 1866 (6): 165759.
22. Sykes D B, Kfoury Y S, Mercier F E, et al. Inhibition of Dihydroorotate Dehydrogenase Overcomes Differentiation Blockade in Acute Myeloid Leukemia. Cell. 2016; 167 (1): 171-186.e115.
23. Zhu F, Rui L. PRMT5 in gene regulation and hematologic malignancies. Genes Dis. 2019; 6 (3): 247-257.
24. He X, Zhu Y, Lin Y C, et al. PRMTI-mediated FLT3 arginine methylation promotes maintenance of FLT3-ITD (+) acute myeloid leukemia. Blood. 2019; 134 (6): 548-560.
25. Ando K, Hamard P-J, Liu F, et al. The AML-Associated FLT3-ITD Kinase Regulates Histone Modifications and Cytokine Signaling, Via Effects on PRMT5. Blood. 2016; 128 (22): 2696-2696.
26. Dhir A, Paterson A J, Qiu S, Mullen A K, Anderson N R, Bhatia R. An Epigenetic Screen Identifies PRMT5 As a Target for Inhibition of FLT3-ITD AML Cell Growth in Combination with Tyrosine Kinase Inhibitors. Blood. 2019; 134 (Supplement_1): 2524-2524.
27. Tibes R, Bogenberger J M. Transcriptional Silencing of MCL-1 Through Cyclin-Dependent Kinase Inhibition in Acute Myeloid Leukemia. Frontiers in Oncology. 2019; 9.
28 Phillips D C, Jin S, Gregory G P, et al. A novel CDK9 inhibitor increases the efficacy of venetoclax (ABT-199) in multiple models of hematologic malignancies. Leukemia. 2020; 34 (6): 1646-1657.
29. Mitra P, Yang R M, Sutton J, Ramsay R G, Gonda T J. CDK9 inhibitors selectively target estrogen receptor-positive breast cancer cells through combined inhibition of MYB and MCL-1 expression. Oncotarget. 2016; 7 (8): 9069-9083.
30. Farge T, Saland E, de Toni F, et al. Chemotherapy-Resistant Human Acute Myeloid Leukemia Cells Are Not Enriched for Leukemic Stem Cells but Require Oxidative Metabolism. Cancer Discov. 2017; 7 (7): 716-735.
31. Gross D N, van den Heuvel A P, Birnbaum M J. The role of FoxO in the regulation of metabolism. Oncogene. 2008; 27 (16): 2320-2336.
32 Amaya M, Inguva A, Pei S, et al. The STAT3-MYC Axis Promotes Survival of Leukemia Stem Cells by Regulating SLC1A5 and Oxidative Phosphorylation. Blood. 2021.
33. Lee K M, Giltnane J M, Balko J M, et al. MYC and MCLI Cooperatively Promote Chemotherapy-Resistant Breast Cancer Stem Cells via Regulation of Mitochondrial Oxidative Phosphorylation. Cell Metab. 2017; 26 (4): 633-647.e637.
34. Gobel A, Rauner M, Hofbauer L C, Rachner T D. Cholesterol and beyond—The role of the mevalonate pathway in cancer biology. Biochim Biophys Acta Rev Cancer. 2020; 1873 (2): 188351.
35. Yamauchi T, Miyawaki K, Semba Y, et al. Targeting leukemia-specific dependence on the de novo purine synthesis pathway. Leukemia. 2021.
36. Bjelosevic S, Gruber E, Newbold A, et al. Serine Biosynthesis Is a Metabolic Vulnerability in FLT3-ITD-Driven Acute Myeloid Leukemia. Cancer Discov. 2021; 11 (6): 1582-1599.
37. Lu X, Han L, Busquets J, et al. The Combined Treatment With the FLT3-Inhibitor AC220 and the Complex I Inhibitor IACS-010759 Synergistically Depletes Wt- and FLT3-Mutated Acute Myeloid Leukemia Cells. Front Oncol. 2021; 11:686765.
38. Lin K H, Xie A, Rutter J C, et al. Systematic Dissection of the Metabolic-Apoptotic Interface in AML Reveals Heme Biosynthesis to Be a Regulator of Drug Sensitivity. Cell Metab. 2019; 29 (5): 1217-1231.e1217.
39. Chen X, Glytsou C, Zhou H, et al. Targeting Mitochondrial Structure Sensitizes Acute Myeloid Leukemia to Venetoclax Treatment. Cancer Discov. 2019; 9 (7): 890-909.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

1. A method of treating FLT3-associated disease in a subject in need thereof, the method comprising exposing an abnormal cell to a FLT3 inhibitor, wherein the method further comprises exposing the abnormal cell to a composition which promotes reactivation of glycolysis of the cell.

2. The method of claim 1, wherein promoting reactivation of glycolysis comprises reducing or inhibiting alternative metabolic pathways.

3. The method of claim 2, wherein the alternative metabolic pathways comprise oxidative phosphorylation, mevalonate metabolism, and/or purine biosynthesis.

4. The method of claim 1, wherein reactivating glycolysis is accomplished by administering to the subject an inhibitor of protein arginine N-methyltransferase 5 (PRMT5), cyclin dependent kinase 9 (CDK9), and/or dihydroorotate dehydrogenase (DHODH).

5. The method of claim 4, wherein an inhibitor of B-cell lymphoma 2 (BCL2) and/or exportin 1 (XPO1) is also given.

6. The method of claim 1, wherein the FLT3-associated disease is caused by an alteration in the FLT3 gene.

7. The method of claim 6, wherein said alteration comprises an internal tandem duplication (ITD) and/or mutations within the tyrosine kinase domain (TKD) and/or the juxtamembrane domain (JMD).

8. The method of claim 1, wherein the FLT3-associated disease is acute myeloid leukemia or acute lymphoblastic leukemia.

9. The method of claim 1, wherein the FLT3 inhibitor is a small molecule.

10. The method of claim 9, wherein the FLT3 inhibitor is gilteritinib.

11. The method of claim 1, wherein the FLT3 inhibitor is interfering RNA.

12. The method of claim 11, wherein the RNA is miRNA or siRNA.

13. The method of claim 1, wherein the FLT3 inhibitor is an antibody.

14. The method of claim 2, wherein inhibiting alternative metabolic pathways comprises using a small molecule.

15. The method of claim 2, wherein inhibiting alternative metabolic pathways comprises using interfering RNA.

16. The method of claim 2, wherein inhibiting alternative metabolic pathways comprises using an antibody as an inhibitor.

17. The method of claim 4, wherein brequinar is used an inhibitor of DHODH.

18. The method of claim 4, wherein dinaciclib is used as an inhibitor of CDK9.

19. The method of claim 4, wherein EPZ015666 is used as an inhibitor or PRMT5.

20. A composition for treating FLT3-associated disease, the composition comprising a combination of a reactivator of glycolysis and an inhibitor of FLT3.

21-34. (canceled)
